(19) United States
(12) Patent Application Publication
(10) Pub. No.: US 2024/0076699 A1
(43) Pub. Date: Mar. 7, 2024

(54) BIOSYNTHESIS OF SUBSTITUTED COMPOUNDS AND CANNABINOIDS

(71) Applicant: Invizyne Technologies, Inc., Monrovia, CA (US)

(72) Inventors: Tyler P. Korman, Sierra Madre, CA (US); Bastian Vogeli, Monrovia, CA (US); Andrew Lingard, Kingston (CA); Mohammad Hayat, Rolling Hills Estates, CA (US)

(73) Assignee: Invizyne Technologies, Inc., Monrovia, CA (US)

(21) Appl. No.: 18/227,719

(22) Filed: Jul. 28, 2023

Related U.S. Application Data
(60) Provisional application No. 63/370,070, filed on Aug. 1, 2022.

(51) Int. Cl. C12P 7/22; C12N 9/10; C12N 9/12

(52) U.S. Cl. CPC C12P 7/22; C12N 9/1085; C12N 9/1205; C12Y 205/0101; C12Y 207/0105; C12Y 207/01127

(57) ABSTRACT

Systems and methods for prenylation and recombinant pathways for the production of compounds, cannabinoids, cannabinoid precursors, and other prenylated chemicals in a cell free system. The present invention further includes enzymes and recombinant microorganisms that catalyze the reaction. The compounds, cannabinoids, cannabinoid precursors, and other prenylated chemicals are operable to be substituted with at least one deuterium, at least one tritium, at least one halogen, at least one hydroxyl group, and/or at least one additional isotope.

### CROSS REFERENCES TO RELATED APPLICATIONS

This application is related to and claims priority from the following U.S. patent applications. This application claims priority to and the benefit of U.S. Provisional Application No. 63/370,070 which is incorporated herein by reference in its entirety.

### REFERENCE TO SEQUENCE LISTING

The official copy of the Sequence Listing is submitted concurrently with the specification as an XML formatted file via EFS-Web, with a file name of “4482003.xml”, a creation date of Jul. 28, 2023, and a size of 133 kilobytes. The Sequence Listing filed via EFS-Web is part of the specification and is incorporated in its entirety by reference herein.

### BACKGROUND OF THE INVENTION

### 1. Field of the Invention

The present invention relates to cannabinoids, and more specifically to substituted compounds and cannabinoids, and biosynthesis of the same.

### 2. Description of the Prior Art

Prenylation of natural compounds adds structural diversity, alters biological activity, and enhances therapeutic potential. Prenylated compounds often have low natural abundance or are difficult to isolate. Some prenylated natural products include a large class of bioactive molecules with demonstrated medicinal properties. Examples include prenyl-flavanoids, prenyl-stilbenoids, and cannabinoids.

Cannabinoids are a large class of bioactive plant derived natural products that regulate the cannabinoid receptors (CB1 and CB2) of the human endocannabinoid system. Cannabinoids are promising pharmacological agents with over 100 ongoing clinical trials investigating their therapeutic benefits as antiemetics, anticonvulsants, analgesics, and antidepressants. Further, three cannabinoid therapies have been FDA approved to treat chemotherapy induced nausea, MS spasticity and seizures associated with severe epilepsy.

Despite their therapeutic potential, the production of pharmaceutical grade (>99%) cannabinoids still presents major technical challenges. Cannabis plants like marijuana and hemp produce high levels of tetrahydrocannabinolic acid (THCA) and cannabidiolic acid (CBDA), along with a variety of lower abundance cannabinoids. However, even highly expressed cannabinoids like CBDA and THCA, are challenging to isolate due to the high structural similarity of contaminating cannabinoids and the variability of cannabinoid composition with each crop. These problems are magnified when attempting to isolate rare cannabinoids. Moreover, current Cannabis farming practices present serious environmental challenges. Consequently, there is considerable interest in developing alternative methods for the production of cannabinoids and cannabinoid analogs.

Further, it is generally known in the prior art to synthesize deuterated cannabinoids.

Prior art documents include the following:

U.S. Pat. No. 5,036,014 for Deuterated cannabinoids as standards for the analysis of tetrahydrocannabinol and its metabolites in biological fluids by inventors Elsohly, et al., filed Jan. 31, 1989 and issued Jul. 30, 1991, is directed to new internal standards for use in gas chromatography/mass spectrometry test methods, comprising deuterated cannabinoids, which have been developed for the analysis of tetrahydrocannabinol and its metabolites in biological fluids.

U.S. Pat. No. 5,633,357 for Synthesis of carboxylic acid glucuronides by inventors Tius, et al., filed Mar. 4, 1994 and issued May 27, 1997, is directed to a method of producing a carboxylic acid glucuronide by reacting a carboxylic acid precursor with a blocked sugar epoxide precursor. Also disclosed are: deuterated 11-nor-Δ8- or Δ9-THC carboxylic acid glucuronide having a deuterated hydrocarbon chain; 5′-deuterated 11-nor-Δ8- or Δ9-THC-carboxylic acid or 5′-deuterated Δ8- or Δ9-THC glucuronide. The compositions are useful as GC-MS standards; in methods for preparing antibodies reactive with a THC glucuronide; and, in GC-MS diagnostic methods for THC metabolites.

U.S. Pat. No. 10,837,031 for Recombinant production systems for prenylated polyketides of the cannabinoid family by inventors Barr, et al., filed May 10, 2018 and issued Nov. 22, 2018, is directed to production methods, enzymes and recombinant yeast strains for the biosynthesis of clinically important prenylated polyketides of the cannabinoid family. Using readily available starting materials, heterologous enzymes are used to direct cannabinoid biosynthesis in yeast.

WIPO Publication No. WO2021034403 for Cannabinoid acid ester compositions and uses thereof by inventors Swisa, et al., filed Jun. 19, 2020 and published Feb. 25, 2021, is directed to pharmaceutical compositions including a cannabinoid acid ester compound alone or in combination with one or more additional cannabinoid compounds. The publication discloses that the cannabinoid acid ester compound is a tetrahydrocannabinolic acid (THCA) ester. The publication also discloses that the cannabinoid acid ester compound is a cannabigerolic (CBGA) acid ester. The publication also discloses that the cannabinoid acid ester compound is a cannabinolic (CBNA) acid ester. A variety of therapeutic applications in which the cannabinoid acid ester compounds and pharmaceutical compositions find use are also provided, including combination therapies using cannabinoid acid ester compounds and one or more additional therapeutic agents.

WIPO Publication No. WO2020186010 for Cannabinoid acid ester compositions and uses thereof by inventors Robinson, et al., filed Mar. 12, 2020 and published Sep. 17, 2020, is directed to pharmaceutical compositions including a cannabinoid acid ester compound alone or in combination with one or more additional cannabinoid compounds. The publication discloses that the cannabinoid acid ester compound is a cannabidiolic acid ester. A variety of therapeutic applications in which the cannabinoid acid ester compounds and pharmaceutical compositions find use are also provided, including combination therapies using cannabinoid acid ester compounds and one or more additional therapeutic agents.

U.S. Publication No. 20210403408 for Cannabinoid analogs and methods for their preparation by inventors Barr, et al., filed Apr. 29, 2021 and published Jun. 30, 2021, is directed to cannabinoid analogs, including halogenated cannabinoid analogs, hydroxylated cannabinoid analogs, deuterated cannabinoid analogs, and tritiated cannabinoid analogs. The cannabinoid analogs can be prepared by partial or total expression in modified host cells, such as recombinantly modified yeast cells, optionally in combination with chemical synthetic steps

U.S. Publication No. 20210230113 for Cannabinoid derivatives by inventor Filer, filed Jan. 22, 2021 and published Jul. 29, 2021, is directed to 8,9-dihydrocannabinoid derivatives, deuterated cannabinoid derivatives, and tritiated cannabinoid derivatives. The disclosure also provides compositions, methods of use, and processes of preparation of the foregoing derivatives.

WIPO Publication No. WO2021000053 for Cannabinoid derivatives by inventors Omeara, et al., filed Jul. 3, 2020 and published Jan. 7, 2021, is directed to cannabinoid derivatives, a pharmaceutical composition comprising said derivative and a method of using said derivatives in treating or preventing a disease associated with cannabinoid receptors. The claimed cannabinoid derivatives are described by the following formula or an enantiomer, diastereomer, racemate, tautomer, or metabolite thereof, or a pharmaceutically acceptable salt, solvate or hydrate of the compound.

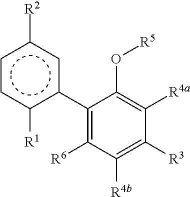

WIPO Publication No. WO2021046640 for Cannabinoid derivatives and precursors, and asymmetric synthesis for same by inventors Abdur-Rashid, et al., filed Sep. 9, 2020 and published Mar. 18, 2021, is directed to new cannabinoid derivatives and precursors and catalytic asymmetric processes for their preparation. The disclosure also relates to pharmaceutical compositions and pharmaceutical and analytical uses of the new cannabinoid derivatives. For instance, the disclosure relates to the preparation of new precursors, and the use of such precursor compounds for the preparation of isotope labelled cannabinoid products using chiral and achiral catalysts and catalytic processes. The deuterium, carbon-13 and carbon-14 containing compounds can be prepared and purified prior to transformation to the desired individual deuterated cannabinoid products.

WIPO Publication No. WO2021113669 for Cannabinoids and uses thereof by inventors Deng, et al., filed Dec. 4, 2020 and published Jun. 10, 2021, is directed to cannabinoid compounds, pharmaceutical compositions including one or more cannabinoid compounds, and the use of pharmaceutical compositions including one or more cannabinoid compounds for the treatment of a disease or condition (e.g., a fibrotic disease or an inflammatory disease) in a subject in need thereof.

WIPO Publication No. WO2022082313 for Compositions and methods for treating neuronal disorders with cannabinoids by inventors Hsu, et al., filed Oct. 21, 2021 and published Apr. 28, 2022, is directed to methods and compositions comprising a cannabinoid compound for providing neuroprotection and/or stimulating neuritogenesis. The cannabinoid compound can be a compound as shown below, wherein R1 is COOH or H, R2 is C3H7 or C5H11, R3 is H or Me, R4 and R5 are Me or (CH2)2CH═C(CH3)2, such as CBGA, a derivative thereof, a prodrug thereof, or combinations thereof, and can be used in the treatment of neurodegenerative diseases, or to promote neurite elongation and/or restore neurite formation in patients in need thereof.

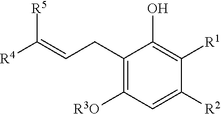

WIPO Publication No. WO2021150636 for Genetically modified yeast for the production of cannabigerolic acid, cannabichromenic acid and related cannabinoids by inventors Barr, et al., filed Jan. 20, 2021 and published Jul. 29, 2021, is directed to production methods, enzymes and recombinant yeast strains for the biosynthesis of clinically important cannabinoid compounds.

U.S. Publication No. 20100298579 for Process for preparing synthetic cannabinoids by inventors Steup, et al., filed Apr. 29, 2010 and published Nov. 25, 2010, is directed to organic synthesis, more particularly a process for preparing cannabinoids. The process described is applicable to all stereoisomers and homologues of cannabinoids. For this purpose, the publication provides a process for preparing the abovementioned compounds in two or three chemical synthesis steps.

U.S. Publication No. 20100152283 for Tetrahydrocannabinol modulators of cannabinoid receptors by inventors Gant, et al., filed Dec. 17, 2009 and published Jun. 17, 2010, is directed to tetrahydrocannabinol modulators of cannabinoid receptors, pharmaceutical compositions thereof, and methods of use thereof.

WIPO Publication No. WO2020102430 for Use of type I and type II polyketide synthases for the production of cannabinoids and cannabinoid analogs by inventors Barr, et al., filed Nov. 13, 2019 and published May 22, 2020, is directed to production methods, enzymes and recombinant yeast strains for the biosynthesis of clinically important prenylated polyketides of the cannabinoid family. Using readily available starting materials, heterologous enzymes are used to direct cannabinoid biosynthesis in yeast.

U.S. Publication No. 20210040512 for Recombinant production systems for prenylated polyketides of the cannabinoid family by inventors Barr, et al., filed Oct. 21, 2020 and published Feb. 11, 2021, is directed to production methods, enzymes and recombinant yeast strains for the biosynthesis of clinically important prenylated polyketides of the cannabinoid family. Using readily available starting materials, heterologous enzymes are used to direct cannabinoid biosynthesis in yeast.

WIPO Publication No. WO2021133989 for Preparation of cannabichromene and related cannabinoids by inventor Marlowe, filed Dec. 23, 2020 and published Jul. 1, 2021, is directed to methods for the production of cannabichromene and related cannabinoid compounds. The methods include: forming a reaction mixture comprising 3,7-dimethylocta-2,6-dienal, a diamine, and olivetol or a related starting material; and maintaining the reaction mixture under conditions sufficient to form the desired product. Methods of the present disclosure may also include one-pot conversion of cannabichromene-type products to cannabinol-type products.

WIPO Publication No. WO2021102567 for Cannabigerol derivatives and use thereof as cannabinoid receptor modulators by inventors Ahmar, et al., filed Nov. 25, 2020 and published Jun. 3, 2021, is directed to synthesis of a range of pentylbezene-1,3-diol compounds with the formula shown below. These compounds bind to cannabinoid 1 and 2 receptors (CB1 and CB2), and are thus presumed to be useful in modulating the activity of such receptors. Accordingly, the use of such synthetic cannabinoids in the treatment of various disorders mediated by CB1 and CB2 is contemplated.

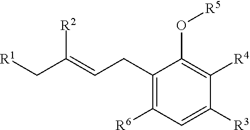

WIPO Publication No. WO2021050786 for Cannabinoid compositions with improved organoleptic and therapeutic properties, method of production, and use thereof by inventors Alarcon, et al., filed Sep. 10, 2020 and published Mar. 18, 2021, is directed to compositions comprising cannabinoids, terpenoids, and other flavonoids. Also provided herein are methods for producing compositions comprising cannabinoids, terpenoids, and other flavonoids at industrial scale. The compositions provided in the disclosure possess desirable organoleptic properties and therapeutic effects when ingested or topically applied. The compositions of the disclosure are useful in inhalable, ingestible, or topical products to relieve and treat various acute or chronic illnesses.

WIPO Publication No. WO2021222288 for Compositions and methods for enhancing recombinant biosynthesis of cannabinoids by inventors Feng, et al., filed Apr. 27, 2021 and published Nov. 4, 2021, is directed to recombinant host cells comprising a pathway capable of producing a cannabinoid and a heterologous nucleic acid that encodes a protein not in the pathway that enhances the host cells' ability to produce the cannabinoid. The disclosure also provides methods of using host cells to produce cannabinoids.

WIPO Publication No. WO2021195517 for Compositions and methods for recombinant biosynthesis of cannabinoids by inventor Schuetz., filed Mar. 26, 2021 and published Sep. 30, 2021, is directed to recombinant host cells comprising a pathway capable of producing a cannabinoid and a nucleic acid derived from a Cannabis trichome mRNA that that does not encode an enzyme in the pathway but enhances the host cells' ability to produce the cannabinoid. The disclosure also provides methods of using host cells to produce cannabinoids.

U.S. Publication No. 20110311474 for Novel tricyclic compounds by inventors Wishart, et al., filed Dec. 1, 2010 and published Apr. 23, 2013, is directed to compounds shown below, pharmaceutically acceptable salts, pro-drugs, biologically active metabolites, stereoisomers and isomers thereof wherein the variable are defined in the disclosure. The compounds of the disclosure are useful for treating immunological and oncological conditions.

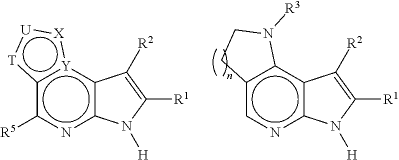

WIPO Publication No. WO2021183448 for Optimized olivetolic acid cyclase polypeptides by inventors Horwitz, et al., filed Mar. 8, 2021 and published Sep. 16, 2021, is directed to engineered variants of an olivetolic acid cyclase polypeptide, wherein the engineered variants comprise an amino acid sequence of SEQ ID NO:1 comprising at least one amino acid substitution, nucleic acids comprising nucleotide sequences encoding said engineered variants, methods of making modified host cells comprising said nucleic acids, modified host cells expressing said engineered variants, methods of producing olivetolic acid, olivetolic acid derivatives, cannabinoids or cannabinoid derivatives, and methods of screening engineered variants of the olivetolic acid cyclase polypeptide.

U.S. Publication No. 20200254041 for Rapid onset and extended action plant-based and synthetic cannabinoid formulations by inventors Leone-Bay, et al., filed Oct. 5, 2018 and published Jun. 2, 2020, is directed to rapid onset and extended action plant-based medicinal compounds or nutritional supplements and synthetic cannabinoid formulations. Rapid onset is provided by N-acylated fatty amino acids and/or penetration enhancers. Extended action can be provided by one or more sustained-release systems.

U.S. Publication No. 20210251947 for Stable formulations of dronabinol by inventor Elkarim, filed Feb. 9, 2021 and published Aug. 19, 2021, is directed to formulations, methods of manufacturing, and methods of treatment using formulations of cannabinoids that are stable at room temperature for at least about one to two years. The publications discloses that the composition is an oxidatively stable formulation of dronabinol.

### SUMMARY OF THE INVENTION

The present invention relates to substituted compounds and cannabinoids, and biosynthesis of the same.

It is an object of this invention to provide substituted compounds and cannabinoids with more favorable pharmacokinetics, and systems and methods for the biosynthesis of the same.

In one embodiment, the present invention provides a cannabinoid composition including a cannabinoid derived from a deuterated fatty acid, wherein the compound includes a deuterated carbon chain, wherein the deuterated carbon chain includes at least one deuterated carbon.

In another embodiment, the present invention provides a method for synthesizing a cannabinoid compound or derivative thereof, including phosphorylating prenol and/or isoprenol using hydroxyethylthiazole kinse (ThiM) to produce isopentenyl phosphate, isomerizing isopentenyl phosphate to produce isopentenyl diphosphate, wherein isopentenyl diphosphate is operable to be phosphorylated in the presence of inositol polyphosphate kinase to produce dimethylallyl diphosphate (DMAPP), synthesizing geranyl pyrophosphate from isopentenyl diphosphate and/or DMAPP in the presence of farnesyl pyrophosphate synthase, activating a deuterated fatty acid to produce a deuterated CoA thioester, activating an acid to produce a second CoA thioester, synthesizing the deuterated CoA thioester and the second CoA thioester, cyclizing the synthesized product to produce a deuterated olivetolic acid, prenylating the deuterated olivetolic acid in the presence of geranyl pyrophosphate to produce a deuterated cannabigerolic acid, and cyclizing the deuterated cannabigerolic acid to produce a deuterated cannabinoid.

In yet another embodiment, the present invention provides a method of cell-free synthesis of a cannabinoid or derivative thereof, including cloning at least one polynucleotide sequence encoding at least one enzyme into at least one microorganism to produce a modified microorganism, wherein the modified microorganism exhibits elevated expression of the at least one enzyme in comparison to an unmodified parental microorganism, lysing at least one cell of the modified microorganism to obtain the at least one enzyme, wherein the at least one enzyme is used in a cannabinoid biosynthesis pathway, producing a cannabinoid or derivative thereof using the cannabinoid biosynthesis pathway, including activating a deuterated fatty acid in the presence of an acyl-activating enzyme (AAE) 3 to produce a deuterated CoA thioester, activating an acid in the presence of a corresponding CoA synthetase to produce a second CoA thioester, synthesizing the deuterated CoA thioester and the second CoA thioester in the presence of olivetol synthase (OLS), cyclizing the synthesized product in the presence of olivetolic acid cyclase (OAC) to produce a deuterated olivetolic acid, prenylating the deuterated olivetolic acid in the presence of geranyl pyrophosphate to produce a deuterated cannabigerolic acid, cyclizing the deuterated cannabigerolic acid to produce a deuterated cannabinoid.

These and other aspects of the present invention will become apparent to those skilled in the art after a reading of the following description of the preferred embodiment when considered with the drawings, as they support the claimed invention.

### BRIEF DESCRIPTION OF THE DRAWINGS

FIG. 1 illustrates metabolic pathways of cannabidiol (CBD).

FIG. 2A depicts an exemplary biosynthetic pathway of the disclosure for the production of prenylnatural products.

FIG. 2B depicts the exemplary biosynthetic pathway of the disclosure for the production of prenylnatural products continued from FIG. 2A.

FIG. 2C illustrates a more detailed view of the pathway shown in FIGS. 2A-2B.

FIG. 3A illustrates activity of pyruvate dehydrogenase (PDH) measured in the presence of various aromatic polyketides and 2% ethanol.

FIG. 3B illustrates a comparison of the final titers achieved with the full pathway utilizing PDH and the PDH bypass system.

FIG. 3C illustrates the amount of 5-prenyl-1,6-DHN and CBGA produced over time with the PDH bypass system using wild type (WT) NphB.

FIG. 3D illustrates results of pathway using NphB, AtaPT, or NovQ prenyltransferase using various aromatic substrates.

FIG. 4A illustrates a model of olivetolate in the active site of WT NphB.

FIG. 4B illustrates results of an activity assay to determine the approximate activity of NphB mutants with olivetolate as the substrate.

FIG. 4D illustrates gas chromatography-mass spectrometry (GC-MS) chromatograms of the full pathway reaction products using a M23 mutant and WT NphB compared to a CBGA standard.

FIG. 5A illustrates a cell-free enzymatic production of cannabinoid precursors from glucose over time.

FIG. 5B illustrates a nonane flow cannabigerolic acid (CBGA) capture system.

FIG. 5C illustrates production of cannabinoids over time using cannabidiolic acid synthase (CBDAS).

FIG. 6A illustrates a pathway schematic for the MatB transferase path.

FIG. 6B illustrates a pathway schematic for the MdcA transferase path.

FIG. 6C illustrates additional details of exemplary steps in the polyketide module of the pathways shown in FIGS. 6A-6B.

FIG. 7 shows a pathway schematic for the (iso)prenol to geranylpyrophosphate (GPP) paths.

FIG. 8 shows the various canonical (Eukaryotic) and non-canonical (Archael I and II) mevalonate pathways that operable to generate IPP/DMAPP from acetyl-CoA (or mevalonate).

FIG. 9 illustrates Fourier transform mass spectrometry with probe electrospray ionization (FTMS-pESI) spectral data for acetonitrile with 0.1% formic acid as a control.

FIG. 10 illustrates FTMS-pESI spectral data for cannabigerolic acid (CBGA) in positive mode.

FIG. 11 illustrates FTMS-pESI spectral data for CBGA in negative mode.

FIG. 12 illustrates FTMS-pESI spectral data for the control and CBGA in positive mode.

FIG. 13 illustrates FTMS-pESI spectral data for the control and CBGA in positive mode.

FIG. 14 illustrates FTMS-pESI spectral data for deuterated CBGA (dCBGA) in positive mode.

FIG. 15 illustrates FTMS-pESI spectral data for deuterated CBGA in negative mode.

FIG. 16 illustrates additional FTMS-pESI spectral data for deuterated CBGA in positive mode.

FIG. 17 illustrates additional FTMS-pESI spectral data for deuterated CBGA in negative mode.

FIG. 18 illustrates nLC-MS/MS data for CBGA.

FIG. 19 illustrates nLC-MS/MS data for dCBGA.

FIG. 20 illustrates nLC-MS/MS data for a first sample of CBGA after one hour.

FIG. 21 illustrates nLC-MS/MS data for a second sample of CBGA after one hour.

FIG. 22 illustrates nLC-MS/MS data for a third sample of CBGA after one hour.

FIG. 23 illustrates nLC-MS/MS data for a first sample of dCBGA after one hour.

FIG. 24 illustrates nLC-MS/MS data for a second sample of dCBGA after one hour.

FIG. 25 illustrates nLC-MS/MS data for a third sample of dCBGA after one hour.

FIG. 26 illustrates a graph comparing CBGA and dCBGA.

FIG. 27A illustrates one embodiment of a cell-free biosynthesis pathway for a deuterated cannabinoid.

FIG. 27B illustrates one embodiment of a cell-free biosynthesis pathway for a deuterated cannabinoid including a pathway for GPP production.

FIG. 28A illustrates one embodiment of a deuterated methyl compound.

FIG. 28B illustrates an example of methylation of various small molecules using d3-SAM and a methyltransferase.

FIG. 29 illustrates an example of deuterated methylated psilocybin using a specific methyltransferase.

FIG. 30 illustrates an example of deuterated methylated chrysoeriol using a specific methyltransferase.

FIG. 31 illustrates an example of the synthesis of a deuterated glucoside.

FIG. 32 illustrates an HPLC (high-performance liquid chromatography) trace of cell-free production of a plurality of cannabinoids.

FIG. 33 illustrates a bar graph of the progress made during pathway optimization.

FIG. 34A illustrates a bar graph depicting OLS variants and their corresponding level of CBGA production.

FIG. 34B illustrates a bar graph depicting OLS variants and their corresponding level of CBGA production at varying enzyme loads.

FIG. 35 illustrates a time course of CBGA synthesis reaction according to one embodiment of the present invention.

FIG. 36 illustrates an HPLC trace of CBGA and THCA activity according to one embodiment of the present invention.

FIG. 37 illustrates an HPLC trace of THC in toluene according to one embodiment of the present invention.

FIG. 38 illustrates the comparison between the enzymatic activity of S-adenosyl-L-methionine-d3 and S-adenosyl-L-methionine.

### DETAILED DESCRIPTION

The present invention is generally directed to substituted compounds and cannabinoids, and biosynthesis of the same.

In one embodiment, the present invention provides a cannabinoid composition including a cannabinoid derived from a deuterated fatty acid, wherein the compound includes a deuterated carbon chain, wherein the deuterated carbon chain includes at least one deuterated carbon.

In another embodiment, the present invention provides a method for synthesizing a cannabinoid compound or derivative thereof, including phosphorylating prenol and/or isoprenol using hydroxyethylthiazole kinse (ThiM) to produce isopentenyl phosphate, isomerizing isopentenyl phosphate to produce isopentenyl diphosphate, wherein isopentenyl diphosphate is operable to be phosphorylated in the presence of inositol polyphosphate kinase to produce dimethylallyl diphosphate (DMAPP), synthesizing geranyl pyrophosphate from isopentenyl diphosphate and/or DMAPP in the presence of farnesyl pyrophosphate synthase, activating a deuterated fatty acid to produce a deuterated CoA thioester, activating an acid to produce a second CoA thioester, synthesizing the deuterated CoA thioester and the second CoA thioester, cyclizing the synthesized product to produce a deuterated olivetolic acid, prenylating the deuterated olivetolic acid in the presence of geranyl pyrophosphate to produce a deuterated cannabigerolic acid, and cyclizing the deuterated cannabigerolic acid to produce a deuterated cannabinoid.

In yet another embodiment, the present invention provides a method of cell-free synthesis of a cannabinoid or derivative thereof, including cloning at least one polynucleotide sequence encoding at least one enzyme into at least one microorganism to produce a modified microorganism, wherein the modified microorganism exhibits elevated expression of the at least one enzyme in comparison to an unmodified parental microorganism, lysing at least one cell of the modified microorganism to obtain the at least one enzyme, wherein the at least one enzyme is used in a cannabinoid biosynthesis pathway, producing a cannabinoid or derivative thereof using the cannabinoid biosynthesis pathway, including activating a deuterated fatty acid in the presence of an acyl-activating enzyme (AAE) 3 to produce a deuterated CoA thioester, activating an acid in the presence of a corresponding CoA synthetase to produce a second CoA thioester, synthesizing the deuterated CoA thioester and the second CoA thioester in the presence of olivetol synthase (OLS), cyclizing the synthesized product in the presence of olivetolic acid cyclase (OAC) to produce a deuterated olivetolic acid, prenylating the deuterated olivetolic acid in the presence of geranyl pyrophosphate to produce a deuterated cannabigerolic acid, cyclizing the deuterated cannabigerolic acid to produce a deuterated cannabinoid.

The present invention includes substituted compounds and cannabinoids that have reduced metabolism and similar pharmacodynamics with respect to their unsubstituted analogs. The present invention further includes a cell-free system and method for the biosynthesis of these substituted compounds and cannabinoids. The present invention also includes high-purity, substituted compounds and cannabinoids synthesized by the cell-free method.

None of the prior art discloses a completely extra-cellular system of synthesis of substituted compounds and cannabinoids that provides both high isomeric purity and high absolute purity. The high isomeric purity is due to the regiospecificity of the prenylation enzyme of the present invention, and the high absolute purity is due to the completely cell-free synthesis and the stochiometric efficiency of the systems of the present invention, which result in fewer extraneous compounds to remove and less leftover reagents. Advantageously, greater isometric purity is important when producing substituted byproducts that are not readily metabolized. Further, none of the prior art provides a one-pot system of cell-free synthesis that provides high purity substituted compounds and cannabinoids.

Referring now to the drawings in general, the illustrations are for the purpose of describing one or more preferred embodiments of the invention and are not intended to limit the invention thereto.

Compounds

The present invention includes compounds and cannabinoids substituted with at least one deuterium, at least one tritium, at least one halogen (e.g., fluorine, chlorine, bromine, iodine), at least one hydroxyl group, and/or at least one additional isotope (e.g., ¹¹C, ¹³C, ¹⁴C, ¹³N, ¹⁵N, ¹⁸O, ¹⁷O, ¹⁸O, ³¹P, ³²P, ³⁵S, ¹⁸F, ³⁶Cl). In one embodiment, the at least one tritium and/or the at least one additional isotope is radioactive (e.g., ³H, ¹⁴C). Advantageously, the compounds substituted with at least one radioactive isotope (e.g., ³H, ¹⁴C) is operable to be used in assays for drug and/or substrate tissue distribution. An additional advantage is that the compounds substituted with a positron emitting isotope (e.g., ¹¹C, ¹⁸F, ¹⁵O, ¹³N) are operable to be used in Positron Emission Topography (PET) studies.

The present invention includes cannabinoids, cannabinoid precursors, and other prenylated chemicals substituted with at least one deuterium, at least one tritium, at least one halogen (e.g., fluorine, chlorine, bromine, iodine), at least one hydroxyl group, and/or at least one additional isotope (e.g., ¹¹C, ¹³C, ¹⁴C, ¹³N, ¹⁵N, ¹⁸O, ¹⁷O, ¹⁵O, ³¹P, ³²P, ³⁵S, ¹⁸F, ³⁶Cl). In one embodiment, the at least one tritium and/or the at least one additional isotope is radioactive (e.g., ³H, ¹⁴C). Advantageously, the cannabinoids, the cannabinoid precursors, or the other prenylated chemicals substituted with at least one radioactive isotope (e.g., ³H, ¹⁴C) is operable to be used in assays for drug and/or substrate tissue distribution. An additional advantage is that the cannabinoids, the cannabinoid precursors, or the other prenylated chemicals substituted with a positron emitting isotope (e.g., ¹¹C, ¹⁸F, ¹⁵O, ¹³N) are operable to be used in Positron Emission Topography (PET) studies.

A further advantage of cannabinoids, cannabinoid precursors, and other prenylated chemicals substituted with at least one deuterium is that deuterium is safe, stable, and not radioactive. Deuterium forms stronger bonds with carbon than hydrogen, which may positively impact the absorption, distribution, metabolism, and/or excretion properties of the cannabinoids, the cannabinoid precursors, or the other prenylated chemicals. Further, because deuterium is similar to hydrogen, substitution of deuterium for hydrogen is not expected to impact the synthetic selectivity of the cannabinoid precursors, or the other prenylated chemicals.

In one embodiment, the cannabinoid, the cannabinoid precursor, and/or the other prenylated chemical includes a cannabidiol (CBD), a tetrahydrocannabinol (THC), a cannabinol (CBN), a cannabigerol (CBG), a cannabichromene (CBC), a cannabicyclol (CBL), a cannabinodiol (CBND), a cannabitriol (CBT), a tetrahydrocannabivarin (THCV), a cannabidivarin (CBDV), a cannabigerovarin (CBGV), a cannabigerophorol (CBGP), a tetrahydrocannabiphorol (THCP), a cannabidiphorol (CBDP), a cyclolavandulyl pyrophosphate (CLPP), derivatives thereof, acids thereof, and/or esters of the acids thereof. Examples of cyclolavandulyl derivates are found in U.S. Provisional Patent Application No. 63/333,670, filed Apr. 22, 2022, which is incorporated herein by reference in its entirety.

For example, and not limitation, in one embodiment, the cannabinoid, the cannabinoid precursor, and/or the other prenylated chemical includes at least one deuterium to form a deuterated cannabinoid, a deuterated cannabinoid precursor, or a deuterated other prenylated chemical. The deuterated cannabinoid, the deuterated cannabinoid precursor, or the deuterated other prenylated chemical is operable to improve bioavailability and slow metabolism relative to a non-deuterated cannabinoid, cannabinoid precursor, or the other prenylated chemical.

In one embodiment, the cannabinoid, the cannabinoid precursor, and/or the other prenylated chemical includes at least 10% substitution at a designated substitution site (e.g., 15%), at least 20% substitution at a designated substitution site (e.g., 25%), at least 30% substitution at a designated substitution site (e.g., 35%), at least 40% substitution at a designated substitution site (e.g., 45%), at least 50% substitution at a designated substitution site (e.g., 55%), at least 60% substitution at a designated substitution site (e.g., 65%), at least 70% substitution at a designated substitution site (e.g., 75%), at least 80% substitution at a designated substitution site (e.g., 85%), at least 90% substitution at a designated substitution site (e.g., 95%).

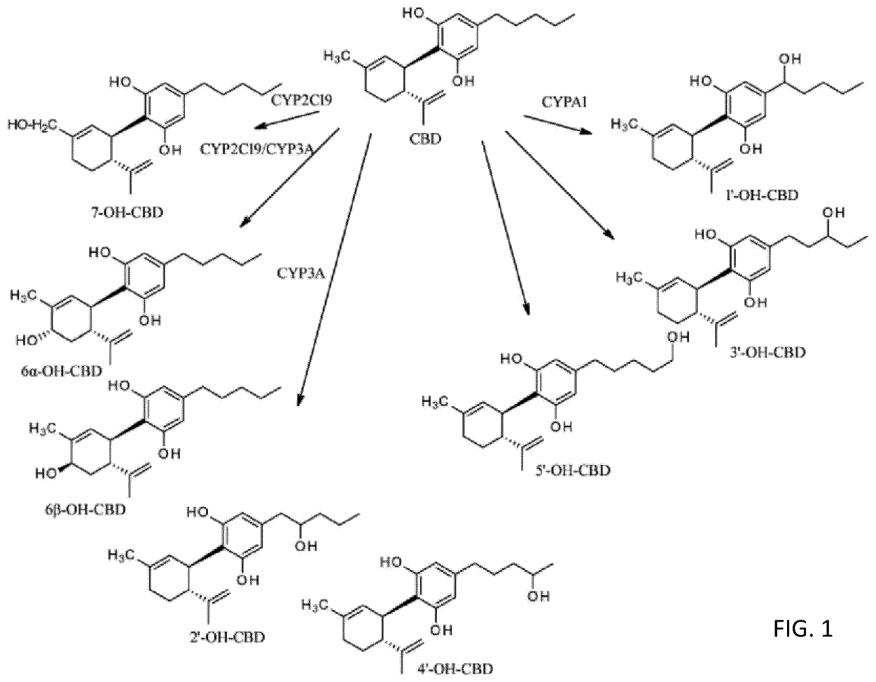

FIG. 1 illustrates metabolic pathways of CBD. As is shown in FIG. 1, CBD undergoes hydroxylation by metabolic enzymes, generally at key metabolic sites. One of the key metabolic sites is the 5′ pentyl tail. Cleavage of the 5′ pentyl tail occurs in ˜25% of metabolites, resulting in an inactive metabolite. The numbering system of CBD and CBDA is shown below.

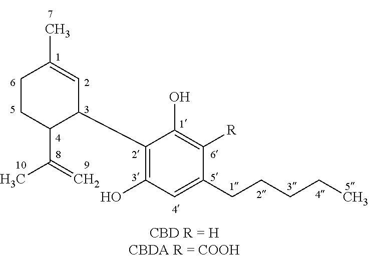

In one embodiment, the present invention includes a CBD molecule with at least one deuterium on the 5′ pentyl tail. Advantageously, replacing hydrogen with deuterium, tritium, and/or halogens slows down cannabinoid metabolism by preventing the hydroxylation shown in FIG. 1. In one embodiment, the 5′ pentyl tail is completely substituted with deuterium, tritium, and/or halogens. Alternatively, the 5′ pentyl tail is partially substituted with deuterium, tritium, and/or halogens.

However, substitution of the terminus of the group on the 5′ pentyl tail may affect the affinity of the pentyl group for the various cannabinoid receptors. In one embodiment, the terminus of the pentyl group is not substituted. Therefore, in one embodiment, the hydrogens of the pentyl chain are substituted on C₁ through C₄ with deuterium, tritium, and/or halogens. In another embodiment, the pentyl chain hydrogens are substituted on C₁ through C₃ with deuterium, tritium, and/or halogens. Alternatively, the pentyl chain hydrogens are substituted on C₁ through C₂ with deuterium, tritium, and/or halogens. In yet another embodiment, the pentyl chain is substituted on C₁ with deuterium, tritium, and/or halogens.

Alternatively, the 5′ group is an alkyl group partially or completely substituted with deuterium, tritium, and/or halogens. The alkyl group includes, but is not limited to, a methyl group, an ethyl group, a propyl group (e.g., n-propyl, isopropyl), a butyl group (e.g., n-butyl, sec-butyl, t-butyl), a hexyl group, a heptyl group, an octyl group, a cycloalkane group (e.g., cyclopropane, cyclobutane, cyclopentane, cyclohexane, etc.), a branched group (e.g., isopropyl, t-butyl, etc.), and/or an aromatic group (e.g., benzyl, coumaryl, etc.). In one embodiment, the alkyl group is not substituted on the terminus. None of the prior art teaches not substituting the alkyl group on the terminus. In another embodiment, the alkyl group is not substituted on the two terminal carbons, on the three terminal carbons, or on the four terminal carbons.

In another embodiment, C₇ is substituted with deuterium, tritium, and/or halogens.

In one embodiment, the compound or cannabinoid is substituted with at least one hydroxyl group. In one embodiment, the at least one hydroxyl group is used to add a boronic acid or ester group. See, e.g., (1) Maslah H, Skarbek C, Pethe S, Labruere R. Anticancer boron-containing prodrugs responsive to oxidative stress from the tumor microenvironment. Eur J Med Chem. 2020 Dec. 1; 207: 112670. doi: 101016/j.ejmech.2020.112670. Epub 2020 Aug. 5. PMID: 32858470; (2) Silva M P, Saraiva L, Pinto M, Sousa M E. Boronic Acids and Their Derivatives in Medicinal Chemistry: Synthesis and Biological Applications. Molecules. 2020 Sep. 21; 25(18):4323. doi 10.3390/molecules25184323. PMID: 32967170; PMCID: PtC7571202; and (3) Liederer B M, Borchardt R T. Enzymes involved in the bioconversion of ester-based prodrugs. J Pharm Sci. 2006 June; 95(6):1177-95. doi: 10.1002/jps.20542. PMID: 16639719, each of which is incorporated herein by reference in its entirety.

In one embodiment, the compound or cannabinoid includes a prodrug. In one embodiment, the prodrug is substituted with at least one deuterium, at least one tritium, at least one halogen (e.g., fluorine, chlorine, bromine, iodine), at least one hydroxyl group, and/or at least one additional isotope (e.g., ¹¹C, ¹³C, ¹⁴C, ¹³N, ¹⁵N, ¹⁸C, ¹⁷O, ¹⁵O, ³¹P, ³²P, ³⁵S, ¹⁸F, ³⁶Cl). I In one embodiment, the prodrug includes a depressant (e.g., codeine, diazepam, benzobarbital, hydrocodone, morphine, oxycodone, tramadol, ethymorphine), a cannabinoid (e.g., THC, THC-0-acetate, THC-O-phosphate, 11-hydroxy-THC), a deliriant (e.g., ibotenic acid), a dissociative (e.g., dextrornethorphan, ketamine), a psychedelic (e.g., IA-LSD, 1B-LSD, IP-ETH-LAD, IP-LSD, bufotenine, ethocybin, MDMA, MIDA, norpsilocin, noribogaine, psilocybin, a nootropic (e.g., 5-HTP, adrafinil, moberacetam), a stimulant (e.g., adrafinil, amfetaminil, benzphetamine, droxidopa, fencamine, L-DOPA, levamisole, lisdexamphetamine, nicotine), naloxone, risperidone, sertraline, trazodone, etoperidone, and/or 5-MT. In one embodiment, the prodrug includes Δ9-THC Hemisuccinate, Δ9-Tetrahydrocannabinol-Valine-Hemisuccinate, THC-O-phosphate, THC-O-acetate, cannaboside, 11-hydroxy-THC, and/or a cannabinoid glycoside. See, e.g., (1) Walker, L. A., Harland, E. C., Best, A. M., ElSohly, M. A. (1999). Δ9-THC Lemisuccinate in Suppository Form as an Alternative to Oral and Smoked THC. In: Nahas, G. G., Sutin, K. M., Harvey,)., Agurell, S., Pace, N., Cancro, R. (eds) Marihuana and Medicine. Flumana Press, Totowa, NJ. https://doi.org/10.1007/978-1-59259-710-9_13; (2) ElSohly M A, Stanford D F, Harland E C, Hlikal A H, Walker L A, Little T L Jr, Rider J N, Jones A B. Rectal bioavailability of delta-9-tetrahydrocannabinol from the hemisuccinate ester in monkeys. J Pharm Sci. 1991 October; 80(10):942-5. doi: 10.1002/ips.2600801008. PMI): 1664466, (3) ElSohly M A, Gul W, Walker L A: Pharmacokinetics and Tolerability of Δ9-THC-Hemisuccinate in a Suppository Formulation as an Alternative to Capsules for the Systemic Delivery of Δ9-THC. Med Cannabis Cannabinoids 2018; 1:44-53. doi: 10.1159/000489037; (4) Upadhye S B, Kulkarni S J, Majumdar S, Avery M A, Gul W, ElSohly M A, Repka M A. Preparation and characterization of inclusion complexes of a hemisuccinate ester prodrug of delta9-tetrahydrocannabinol with modified beta-cyclodextrins. AAPS PharmSciTech. 2010 June; 11(2):509-17. doi: 10.1208/s12249-010-9401-4. Epub 2010 Mar. 24. PMID: 20333489; PMCID: PMC2902337; and (5) Adelli G R, Bhagav P, Taskar P, Hingorani T, Pettaway S, Gul W, ElSohly M A, Repka M A, Majumdar S. Development of a Δ9-Tetrahydrocannabinol Amino Acid-Dicarboxylate Prodrug With Improved Ocular Bioavailability. Invest Ophthalmol Vis Sci. 2017 Apr. 1; 58(4):2167-2179. doi: 10.1167/iovs.16-20757. PMID: 28399267; PM(ID: PMC5389743, each of which is incorporated herein by reference in its entirety. See also, e.g., WJPO Publication No. WO2021173130 and U.S. Patent Publication Nos. 20220194916, 20220168428, and 20210379507, each of which is incorporated herein by reference in its entirety.

Synthesis

Prenylation (also known as isoprenylation or lipidation) is the addition of hydrophobic molecules to a protein or chemical compound. It is usually assumed that prenyl groups (3-methylbut-2-en-1-yl) facilitate attachment to cell membranes, similar to lipid anchors like the GPI anchor. Prenyl groups have been shown to be important for protein-protein binding through specialized prenyl-binding domains.

Prenylated natural products are a large class of bioactive molecules with demonstrated medicinal properties. Examples include, but are not limited to, prenyl-flavanoids, prenyl-stilbenoids, and cannabinoids. Plant-derived phenyl compounds are difficult to isolate due to the structural similarity of contaminating molecules, and the variable composition between crops. These challenges are further exacerbated when attempting to isolate low abundance compounds. Many chemical syntheses have been developed to address the challenges associated with making prenylated natural products, but they are generally impractical for drug manufacturing due to the degree of complexity and low yields.

Microbial production is a useful alternative to natural extraction for prenylated natural products, but comes with many challenges such as the need to divert carbon flux from central metabolism and product toxicity to name a few. For example, prenyl-natural products like prenyl-naringenin, prenyl-resveratrol, and cannabidiolic acid (CBDA) are derived from a combination of the metabolic pathways for fatty acid, isoprenoid, and polyketide biosynthesis. So, high-level production requires efficient rerouting of long, essential, and highly regulated pathways. Despite the challenges, many groups have engineered microbes to produce unprenylated polyketides, like naringenin, resveratrol, and olivetolate, but at relatively low levels (110, 391, and 80 mg/L, respectively). Obtaining prenylated products is even more challenging because geranyl-pyrophosphate (GPP) is an essential metabolite that is toxic to cells at moderate concentrations, creating a significant barrier for high-level microbial production.

Cannabinoids in particular show immense therapeutic potential with over 100 ongoing clinical trials as antiemetics, anticonvulsants, antidepressants, and analgesics. Nevertheless, despite the therapeutic potential of prenyl-natural products, their study and use are limited by the lack of cost-effective production methods.

The two main alternatives to plant-based cannabinoid production are organic synthesis and production in a metabolically engineered host (e.g., plant, yeast, or bacteria). Total syntheses have been elucidated for the production of some cannabinoids, such as THCA and CBDA, but they are often not practical for drug manufacturing. Additionally, the synthetic approach is not modular, requiring a unique synthesis for each cannabinoid. A modular approach is operable to be achieved by using the natural biosynthetic pathway.

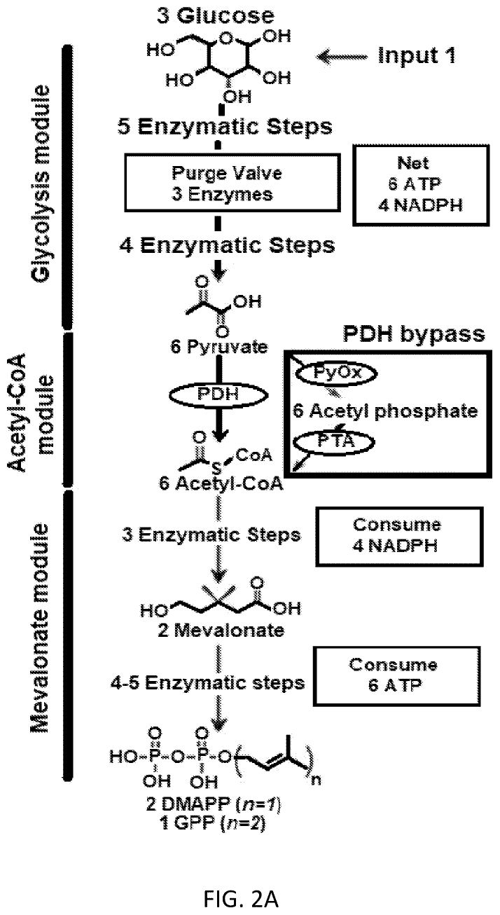

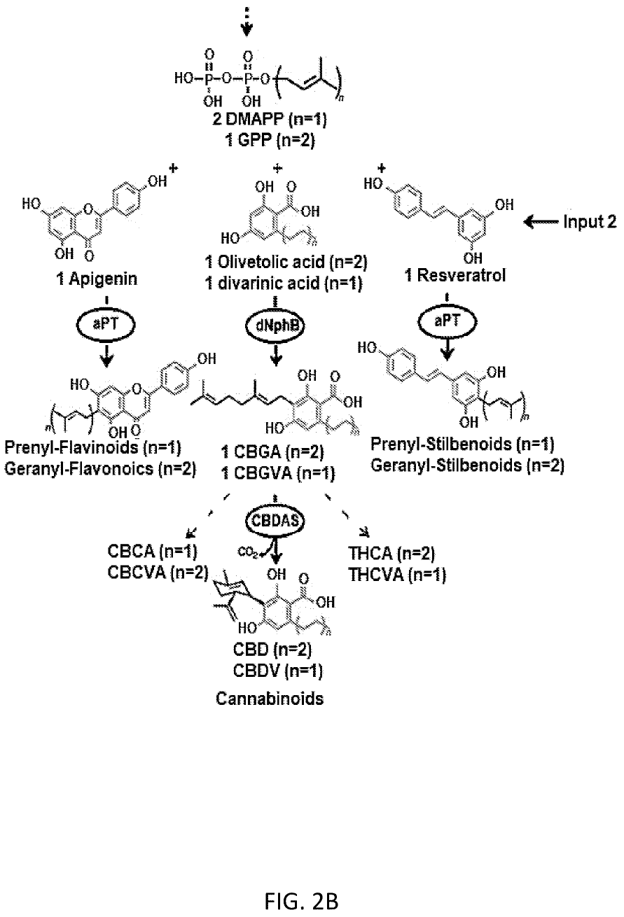

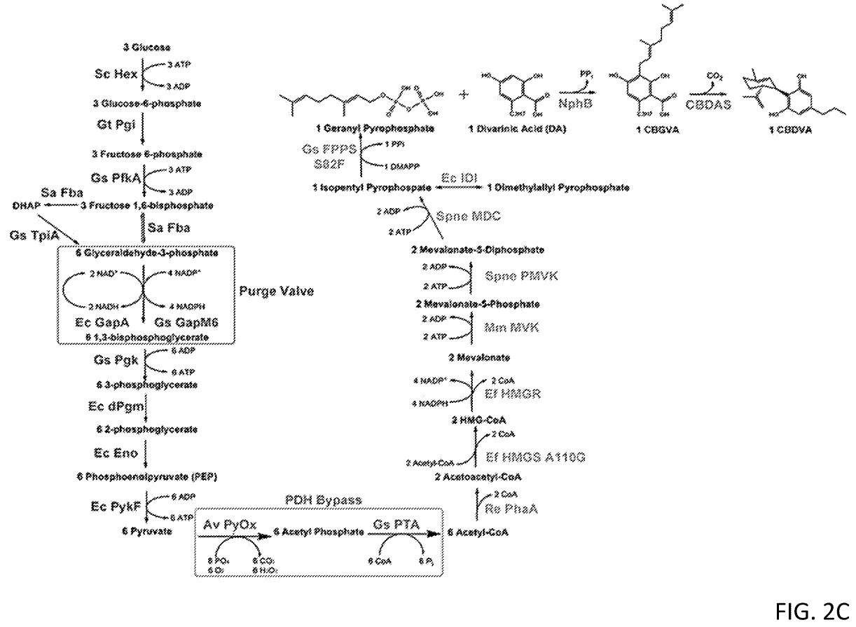

The three major cannabinoids (THCA, CBDA, and cannibichromene) are derived from a single precursor, CBGA. Additionally, three low abundance cannabinoids are derived from cannabierovarinic acid (CBGVA) (FIGS. 2A-2B). Thus, the ability to make CBGA and CBGVA in a heterologous host opens the door to the production of an array of cannabinoids. Unfortunately, engineering microorganisms to produce CBGA and CBGVA has proven extremely challenging.

Cannabinoids are derived from a combination of fatty acid, polyketide, and terpene biosynthetic pathways that generate the key building blocks geranyl pyrophosphate (GPP) and olivetolic acid (OA) (FIGS. 2A-2B). High level CBGA biosynthesis requires the re-routing of long, essential and highly regulated pathways. Moreover, GPP is toxic to cells, creating a notable barrier to high level production in microbes. While Gagne et al. (Proc. Natl. Acad. Sci., 109:12811, 2012, which is incorporated herein by reference in its entirety) engineered a pathway to produce OA in yeast, the titers were very low (0.5 mg L-1), suggesting that high level production of intermediates on the pathway is not straightforward. In a separate study, Zirpel et al. produced THCA in a yeast lysate containing the promiscuous prenyltransferase (NphB) and THCA synthase, supplemented with GPP and olivetolic acid (OA) (J. Biotechnol., 259:204-212, 2017, which is incorporated herein by reference in its entirety). Yet, there are still no published reports of cannabinoid production in engineered live cells from low-cost feedstocks.

Synthetic biochemistry, in which complex biochemical conversions are performed cell-free using a mixture of enzymes, affords potential advantages over traditional metabolic engineering including: a higher level of flexibility in pathway design; greater control over component optimization; more rapid design-build-test cycles; and freedom from cell toxicity of intermediates or products. Advantageously, the present invention provides a cell-free system for the production of cannabinoids.

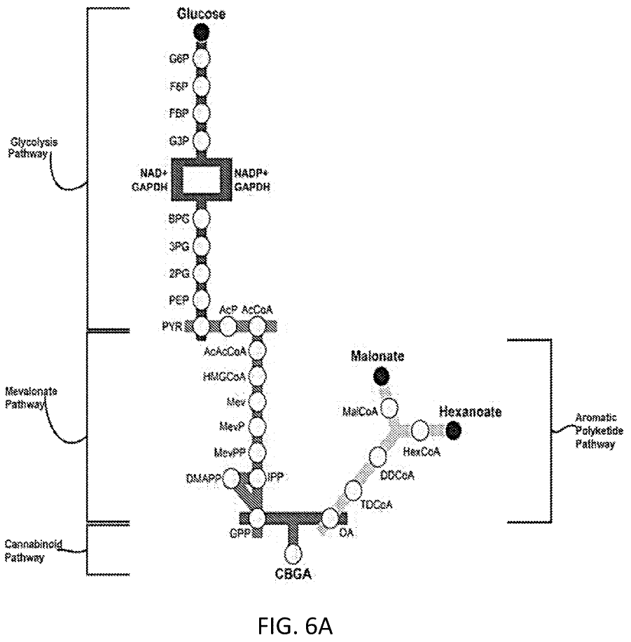

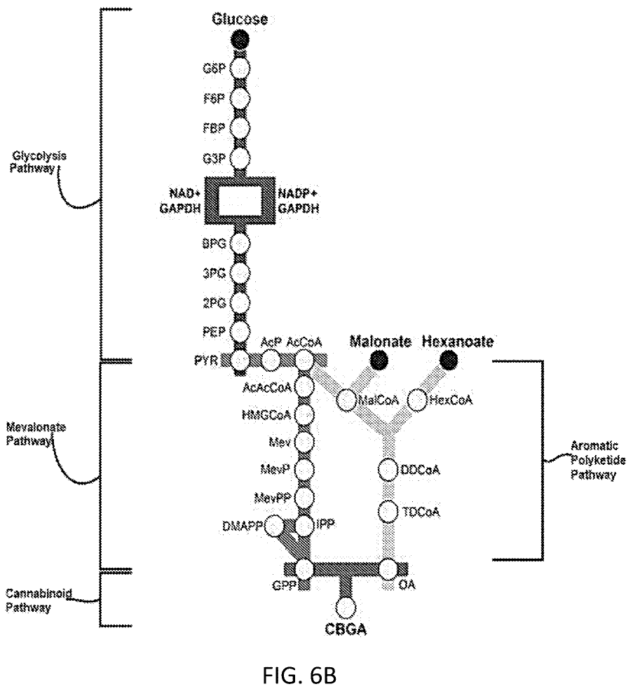

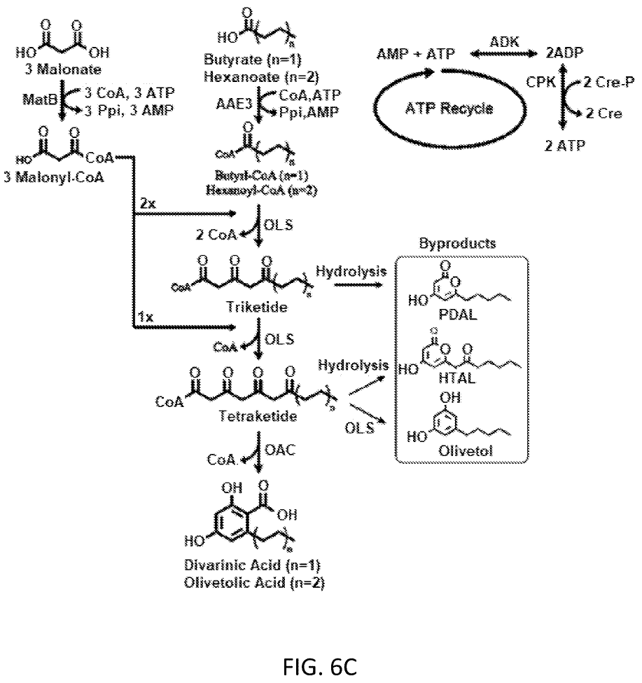

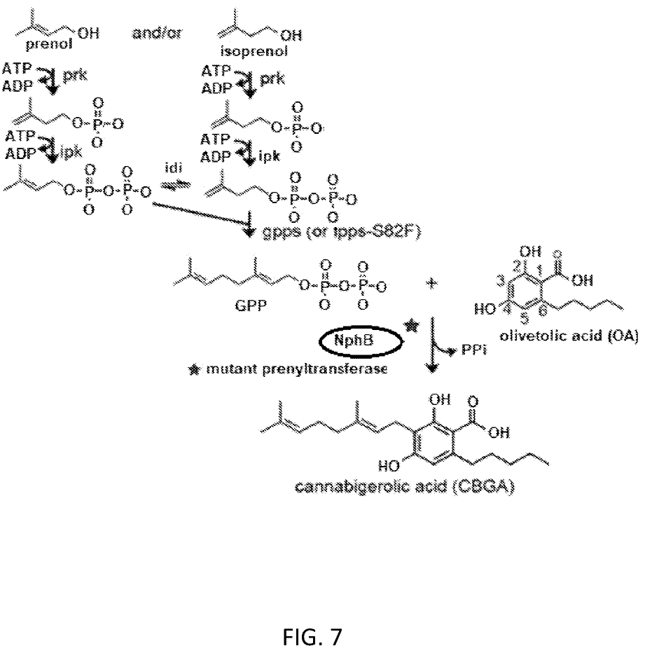

The present invention provides enzyme variants and pathways including such variants for the prenylation of compounds including the production of cannabinoids. In addition, the biosynthetic pathways described herein use “purge valves” to regulate NAD(P)H levels. Such “purge valves” have demonstrated high level production of monoterpenes from glucose indicating that significant GPP is operable to be produced cell-free (see, International Pat. Publ. WO2017/015429, which is incorporated herein by reference in its entirety). These purge valves were used to upgrade and diversify the original system to produce complex natural products like cannabinoids. A synthetic biochemistry approach is outlined in FIGS. 2A-2C, 6A, and 6B. In one embodiment, the disclosure provides a cell-free system for prenylation using GPP derived from glucose (see, FIGS. 2A-2C, 6A, 6B, and 8). In another embodiment, the present invention provides a cell-free system for prenylation using GPP derived from (iso)prenol or prenol (see, FIG. 7). The pathway of FIG. 7 is operable to be coupled to any ATP generating system to produce the ATP needed for a reaction. For example, the pathway is operable to be coupled with a creatine kinase ATP generating system; an acetate kinase system; a glycolysis system as well as others. Enzymes (nucleic acid coding sequences and polypeptides) of FIG. 7 are provided in SEQ ID Nos: 54-65 (e.g., PRK enzymes are provided in SEQ ID Nos: 54-57; IPK enzymes are provided in SEQ ID Nos: 58-61; IDI enzymes are provided in SEQ ID Nos: 62-63; and FPPS enzymes are provided in SEQ ID Nos: 64-65).

NphB is an aromatic prenyltransferase that catalyzes the attachment of a 10-carbon geranyl group to aromatic substrates. NphB exhibits a rich substrate selectivity and product regioselectivity. NphB, identified from Streptomyces, catalyzes the addition of a 10-carbon geranyl group to a number of small organic aromatic substrates. NphB has a spacious and solvent accessible binding pocket in to which two substrate molecules, geranyl diphosphate (GPP) and 1,6-dihydroxynaphthalene (1,6-DHN), are operable to be bound. GPP is stabilized via interactions between its negatively charged diphosphate moiety and several amino acid sidechains, including Lys119, Thr171, Arg228, Tyr216, and Lys284, in addition to Mg²⁺. A Mg²⁺ cofactor is required for the activity of NphB. NphB from Streptomyces has a sequence as set forth in SEQ ID NO:30.

NovQ (accession no. AAF67510, incorporated herein by reference) is a member of the CloQ/NphB class of prenyltransferases. The novQ gene is operable to be cloned from Streptomyces niveus, which produces an aminocoumarin antibiotic, novobiocin. Recombinant NovQ is operable to be expressed in Escherichia coli and purified to homogeneity. The purified enzyme is a soluble monomeric 40-kDa protein that catalyzed the transfer of a dimethylallyl group to 4-hydroxyphenylpyruvate (4-HPP) independently of divalent cations to yield 3-dimethylallyl-4-HPP, an intermediate of novobiocin. In addition to the prenylation of 4-HPP, NovQ catalyzed carbon-carbon-based and carbon-oxygen-based prenylations of a diverse collection of phenylpropanoids, flavonoids, and dihydroxynaphthalenes. Despite its catalytic promiscuity, the NovQ-catalyzed prenylation occurred in a regiospecific manner. NovQ is the first reported prenyltransferase capable of catalyzing the transfer of a dimethylallyl group to both phenylpropanoids, such as p-coumaric acid and caffeic acid, and the B-ring of flavonoids. NovQ is operable to serve as a useful biocatalyst for the synthesis of prenylated phenylpropanoids and prenylated flavonoids.

Aspergillus terreus aromatic prenyltransferase (AtaPT; accession no. AMB20850, incorporated herein by reference), which has recently been discovered and characterized, is responsible for the prenylation of various aromatic compounds. Recombinant AtaPT is operable to be overexpressed in Escherichia co/i and purified. Aspergillus terreus aromatic prenyltransferase (AtaPT) catalyzes predominantly C-monoprenylation of acylphloroglucinols in the presence of different prenyl diphosphates.

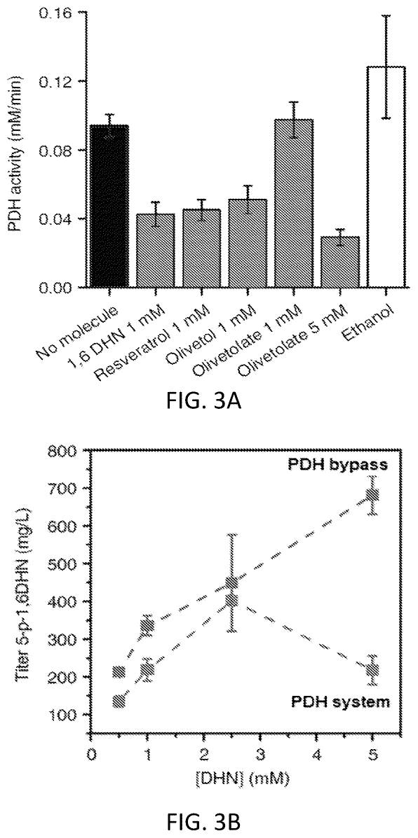

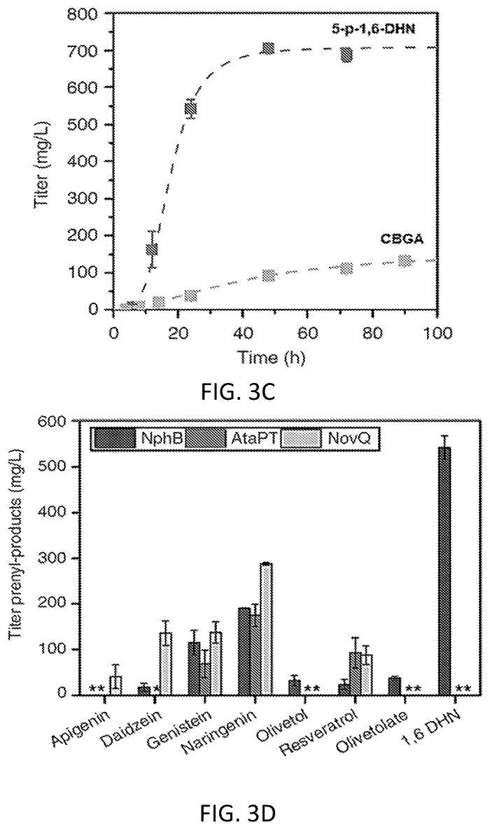

Olivetolic acid (OA) is a relatively poor substrate for wild-type NphB. As a result, the ability of the cell-free system to prenylate a co-substrate was tested by using a more preferred NphB substrate, 1,6 dihydroxynapthalene (1,6 DHN). About 400 mg/L (1.3 mM) of prenylated product was obtained when starting with 2.5 mM 1,6 DHN and 500 mM glucose. However, when the starting 1,6 DHN concentration was increased from 2.5 to 5 mM, final titers decreased 2-fold suggesting that 1,6 DHN was inhibiting one or more enzymes. Enzyme assays revealed that E. coli pyruvate dehydrogenase (EcPDH) was inhibited by not only 1,6 DHN, but several other aromatic polyketides (FIG. 3B). At 1 mM of either 1,6 DHN, olivetol, or resveratrol the activity of PDH decreased 2-fold (FIG. 3B). Thus, experiments were designed to eliminate PDH by implementing a PDH bypass (see FIGS. 2A-2B and 3B). In the PDH bypass, pyruvate was converted to acetyl-CoA using pyruvate oxidase (PyOx) and acetyl-phosphate transferase (PTA) thereby eliminating PDH (FIGS. 2A-2B). As shown in FIG. 3A the new system removed the inhibition seen at higher concentrations of 1,6 DHN and increased titers of 5-prenyl-1,6 DHN 4-fold over the PDH system when starting at 5 mM 1,6 DHN (FIG. 3B). FIG. 3C shows a time course of 5-prenyl-1,6 DHN biosynthesis starting with 5 mM 1,6 DHN utilizing the PDH bypass. Approximately 50% of the 1,6 DHN was converted in the first 24 hours, ultimately reaching a final titer of 705±12 mg/L.

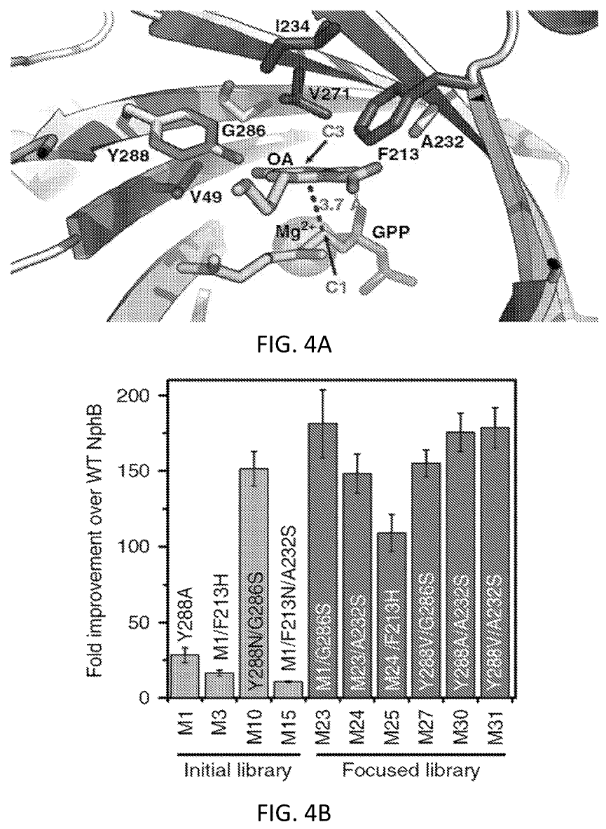

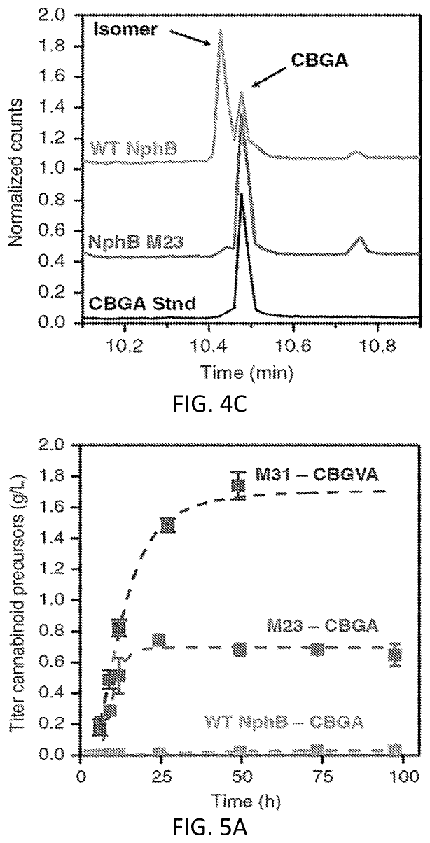

The prenylation of aromatic polyketides by NphB is thought to proceed through a carbocation intermediate in which the first step is dissociation of diphosphate from GPP to create a carbocation on the C1 carbon of GPP, which subsequently attacks a nearby nucleophile. To improve the regiospecificity of prenyl-transfer, OA was modeled into the active site of NphB using the crystal structure of NphB in complex with 1,6 DHN, Mg²⁺ and a nonhydrolyzable analog of GPP (geranyl S-thiolodiphosphate) as a starting point (PDBID 1ZB6; Protein Data Bank reference 1ZB6). For the design, OA was placed into the binding pocket using 1,6 DHN as a guide, situating the desired prenylation site, the C3 carbon of OA, 3.7 Å above the nascent geranyl C1 carbocation (FIG. 4A). The distance chosen was based on the distance of the C5 carbon of 1,6 DHN to the C1 carbon of GPP. Residues in contact with OA were then varied using ROSETTA software to optimize the active site of NphB for binding OA. Side chains in contact with GPP or that potentially provide catalytic function were left fixed. The result was an ensemble of suggested NphB variants.

To reduce the number of variants to test experimentally, changes likely to have the most significant impact on OA binding were ranked using a scoring system. A representative group of variants were picked (Table 1) and each residue was systematically changed back to the wild-type side chain in the background of the other mutations, and the change evaluated in the energy score (Table 2). Y288 replacements had the largest impact on the energy score so Y288A or the Y288N mutation were used in every construct evaluated experimentally. The frequency of mutation, how multiple mutations might work in concert, and the computational energy score to further shape the NphB library were all considered. With these considerations, a library comprised of 29 constructs ranging from a single point mutant to up to 6 mutations per construct was generated as set forth in Table 1 (see also SEQ ID Nos: 1-29; note SEQ ID Nos: 1-29 include a hexahistidine leader from the expression construct, i.e., amino acids 1-20, which are not necessary for biological activity). Table 1 provides exemplary mutations and the fold improvement relative to wild type (i.e., a polypeptide of SEQ ID NO: 30). NphB library constructs and mutations (amino acid positions referenced to SEQ ID NO: 30).

TABLE 1

| NphB | | Fold Improvement |
|---|---|---|
| Construct | Mutations | Over WT |
| M1 | Y288A | 26 |
| M2 | Y288N | 11 |
| M3 | Y288A, F213H | 12 |
| M4 | Y288A, F213N | 2 |
| M5 | Y288N, V49S | 5 |
| M6 | Y288S, V49N | 11 |
| M7 | Y288A, V49S | 9 |
| M8 | Y288N, V49T | 1 |
| M9 | Y288N, I234T | 1 |
| M10 | Y288N, G286S | 150 |
| M11 | Y288N, F213N, V49G | 3 |
| M12 | Y288A, F213N, I234T | 3 |
| M13 | Y288S, F213N, V49N | 2 |
| M14 | Y288N, F213G, I234T | 1 |
| M15 | Y288A, F213N, A232S | 17 |
| M16 | Y288N, F213N, A232S | 2 |
| M17 | Y288N, F213G, V49T | 2 |
| M18 | Y288N, V49S, V271N | 1 |
| M19 | Y288N, F213N, V49S, V271N | 2 |
| M20 | Y288N, F213G, V49T, V271H | 4 |
| M21 | Y288N, F213N, V49S, I234T, A232S, V271N | 0.5 |
| M22 | Y288N, F213G, V49T, I234T, V271H, L298I | 0.5 |
| M23* | Y288A, G286S | 185 |
| M24* | Y288A, G286S, A232S | 150 |
| M25* | Y288A, G286S, A232S, F213H | 110 |
| M27* | Y288V, G286S | 155 |
| M28* | Y288V, G286S, A232S | 1.5 |
| M30* | Y288A, A232S | 175 |
| M31* | Y288V, A232S | 180 |
| M32b | V49I | ND |
| M33b | M162C | ND |
| M34b | M162R | ND |
| M35b | A232N | ND |
| M36b | V271S | ND |
| M37b | V271A | ND |
| M38b | Y288D | ND |
| M39b | Y288H | ND |
| M40b | L298R | ND |
| M41b | L298A | ND |
| M42b | L298G | ND |
| M43b | L298V | ND |
| M44b | L298N | ND |

*Second round focused library.
bMutation predicated by Rosetta, but not tested.
ND—Not determined

Table 2 illustrates kinetic parameters for NphB mutants.

TABLE 2

| Construct | k_{cat} (min⁻¹) | K_{M} (mM) | k_{cat}/K_{M} (min⁻¹ mM⁻¹) |
|---|---|---|---|
| WT NphB | 0.0021 ± 0.00008 | 0.64 ± 0.08 | 0.0033 ± 0.0005 |
| | 0.0047 ± 0.0003^{b} | 0.88 ± 0.2^{b} | 0.005 ± 0.001^{b} |
| NphB M1 | 0.061 ± 0.003 | 0.58 ± 0.11 | 0.11 ± 0.02 |
| NphB M10 | 0.79 ± 0.02 | 0.34 ± 0.02 | 2.4 ± 0.02 |
| NphB M23 | 1.58 ± 0.05 | 0.45 ± 0.05 | 3.5 ± 0.4 |
| | 0.048 ± 0.07^{b} | 2.4 ± 0.6^{b} | 0.2 ± 0.06^{b} |
| NphB M30 | 1.07 ± 0.05 | 0.25 ± 0.05 | 4.2 ± 0.9 |
| NphB M31 | 1.30 ± 0.05 | 0.12 ± 0.02 | 10.8 ± 2.1 |
| | 6.0 ± 0.8^{b} | 1.8 ± 0.5^{b} | 3.3 ± 1^{b} |

^{b}Kinetic parameters for divarinic acid

Recombinant methods for producing and isolating modified NphB polypeptides of the disclosure are described herein. In addition to recombinant production, the polypeptides are operable to be produced by direct peptide synthesis using solid-phase techniques (e.g., Stewart et al. (1969) Solid-Phase Peptide Synthesis (WH Freeman Co, San Francisco); and Merrifield (1963) J. Am. Chem. Soc. 85: 2149-2154; each of which is incorporated herein by reference in its entirety). Peptide synthesis is operable to be performed using manual techniques or by automation. Automated synthesis is operable to be achieved, for example, using Applied Biosystems 431A Peptide Synthesizer (Perkin Elmer, Foster City, Calif.) in accordance with the instructions provided by the manufacturer.

Crudely purified NphB mutants were obtained and an initial screen was performed for CBGA production using GPP and OA at concentrations that were saturating for wild-type NphB. Six constructs were identified that had >10-fold apparent increase in activity (M1, M2, M3, M6, M10, and M15) and 4 constructs that had 2-10-fold apparent improvement (M5, M7, M12, and M20) when compared to WT NphB, while the remaining constructs had similar activity to WT NphB. The top hits from the initial screen (M1, M3, M10, and M15) were purified and more carefully characterized (FIG. 4B). Several observations were apparent from the initial screen: (1) Y288A (M1) and Y288N (M2) by themselves dramatically enhanced activity, as predicted by computation; (2) the presence of Y288N in any construct decreased the purification yield suggesting Y288N may be a destabilizing mutation, making Y288A a more desirable mutation; (3) the addition of G286S in the Y288N (M10) background appeared to improve activity further over Y288N (M2), suggesting that G286S is another favorable mutation; (4) a slight activity improvement of Y288A/F213N/A232S (M15) over Y288A (M1), even though F213N had a neutral or deleterious effect in the Y288A/F213N (M5) construct, suggesting that A232S is also a favorable mutation.

From these initial observations, a focused library was designed that included variants Y288A, GS86S and A232S in various combinations. Other combinations with Y288V were added with the rationale that it may improve stability while still reducing the size of the Y288 side chain. All but one of the constructs in the second library exhibited activity at least 100-fold higher than WT NphB in a one-hour endpoint assay. A comparison of the best mutants from round one and the best mutants from round two are shown in FIG. 4B. Clearly, the combination of beneficial mutations from round 1 improved CBGA production. Additionally, the Y288A and Y288V constructs improved expression of NphB compared to Y288N without sacrificing activity.

The best two mutants from the initial screen were further characterized as well as the best three constructs from the focused library. The kinetic parameters are summarized in Table 2. While all of the mutants have relatively modest effects on Kₘ, a dramatic improvement in k_{cat} values was observed. M23 (the NphB of SEQ ID NO:23) in particular improved k_{cat} 750-fold from 0.0021±0.00008 min-1 to 1.58±0.05 min⁻¹. The catalytic efficiency (k_{cat}/Kₘ) for both M23 and M31 were improved over 1000-fold compared to the wild-type enzyme. Although M31 had a higher k_{cat}/Kₘ than M23, M23 was employed rather than M31 because M23 had a higher k_{cat} and the synthetic biochemistry system generally operates at saturating OA conditions.

The designed mutant M23 not only shows dramatically improved catalytic efficiency for prenylation of OA, it is also extremely specific, producing only the correct CBGA product. WT NphB produces CBGA, but the dominant product is a prenylated isomer (FIG. 4C). In contrast, the designed mutant M23 makes CBGA almost exclusively. Overall, the designed enzyme is a much more effective CBGA synthase than the non-specific prenylating wild-type enzyme.

The disclosure thus provides mutant NphB variants comprising (i) SEQ ID NO:30 and having at least a Y288X mutation, wherein X is A, N, S, V, or a non-natural amino acid; (ii) SEQ ID NO:30 having at least a Y288X mutation, wherein X is A, N, S, V, or a non-natural amino acid, and at least one other mutation selected from V49Z₁, F213Z₂, A232S, I234T, V271Z₃ and/or G286S, wherein Z₁ S, N, T, or G, Z₂ is H, N, or G, and Z₃ is N or H; (iii) any of the mutations combination set forth in Table 1; (iv) any of (i), (ii), or (iii) including from 1-20 (e.g., 2, 5, 10, 15 or 20; or any value between 1 and 20) conservative amino acid substitutions and having NphB activity; (v) a sequence that is at least 85%, 90%, 95%, 98% or 99% identical to SEQ ID NO:1-29 or 30 and which has at least the mutations recited in (i), (ii), or (iii); (vi) an NphB mutation comprising any of the sequence recited in SEQ ID Nos:1-28 or 29 beginning at amino acid 21; or (vii) any sequence that is at least 99% identical to any of SEQ ID Nos: 1-28 or 29 and having NphB activity. “NphB activity” means the ability of the enzyme to prenylate a substrate and more specifically to generate CBGA from OA.

As used herein, a non-natural amino acid refers to amino acids that do not occur in nature such as N-methyl amino acids (e.g., N-methyl L-alanine, N-methyl L-valine, etc.) or alpha-methyl amino acids, beta-homo amino acids, homo-amino acids, and D-amino acids. In a particular embodiment, a non-natural amino acid useful in the disclosure includes a small hydrophobic non-natural amino acid (e.g., N-methyl L-alanine, N-methyl L-valine, etc.).

In addition, the disclosure provides polynucleotides encoding any of the foregoing NphB variants. Due to the degeneration of the genetic code, the actual coding sequences are operable to vary, while still arriving at the recited polypeptide for NphB mutants and variants. Exemplary polynucleotide sequences are provided in SEQ ID nOs: 66, 67, and 68 (corresponding to the polypeptide sequences of SEQ ID NO: 23, 29, and 69, respectively). It will again be readily apparent that the degeneracy of the genetic code allows for wide variation in the percent identity to SEQ ID nOs: 66, 67, and 68, while still encoding a polypeptide of SEQ ID NO: 23, 29, and 69.

The disclosure also provides recombinant host cells and cell free systems including any of the NphB variant enzymes of the disclosure. In some embodiments, the recombinant cells and cell free systems are used carry out prenylation processes. In one embodiment, the system is preferably cell free without any living cells. Alternatively, the system is completely cell free without micelles or other artificial cells, defined as no compartmentalization of any of the reagents, enzymes, or other chemical species in the reaction environment. In one embodiment, the system includes permeabilized cells (e.g., acetone dried yeast). In one embodiment, ATP regeneration is done using the permeabilized cells. In another embodiment, the system includes an engineered heat-treated cell lysate, wherein the biomass (e.g., grown cells) are collected, heated (with or without lysis), and then used. In one embodiment, ATP is regenerated without the use of acetyl-phosphate. In one embodiment, ATP is regenerated using polyphosphate to simplify the biosynthesis pathway. One of ordinary skill in the art will appreciate that the substrate acetyl-phosphate is operable to be used in two competing reactions, which introduces balancing issues in the pathway. The use of polyphosphate in place of acetyl-phosphate addresses and significantly diminishes this risk.

See, e.g., (1) Valliere, M. A., Korman, T. P., Woodall, N. B. et al. A cell-free platform for the prenylation of natural products and application to cannabinoid production. Nat Commun 10, 565 (2019). https://doi.org/10.1038/s41467-019-08448y; (2) Bloemendal V R L J, van Hest J C M, Rutjes F P J T. Synthetic pathways to tetrahydrocannabinol (THC): an overview. Org Biomol Chem. 2020 May 6; 18(17):3203-3215. doi: 10.1039/d0ob00464b. PMID: 32259175; (3) Masanori Asada, Kazuhiro Morimoto, Kazuhiro Nakanishi, Ryuichi Matsuno, Atsuo Tanaka, Akira Kimura, Tadashi Kamikubo, Continuous ATP Regeneration Using Immobilized Yeast Cells, Agricultural and Biological Chemistry, Volume 43, Issue 8, 1 Aug. 1979, Pages 1773-1774, https://doi.org/10.1080/0021369.1979.10863703; and (4) Alissandratos A, Caron K, Loan T D, Hennessy J E, Easton C J. ATP Recycling with Cell Lysate for Enzyme-Catalyzed Chemical Synthesis, Protein Expression and PCR. ACS Chem Biol. 2016 Dec. 16; 11(12):3289-3293. doi: 10.1021/acschembio.6b00838. Epub 2016 Nov. 10. PMID: 27978706, each of which is incorporated herein by reference in its entirety.

One objective of the disclosure is to produce the precursor GPP from glucose or prenol and/or isoprenol, which is then operable to be used to prenylate added OA with a mutant NphB of the disclosure, thereby generating CBGA.

The disclosure thus provides a cell-free system including a plurality of enzymatic steps that converts glucose to geranyl pyrophosphate, wherein the pathway includes a purge valve and a PDH bypass enzymatic process.

As depicted in FIG. 2C, one pathway of the disclosure includes converting glucose to glucose-6-phosphate using a hexokinase. A hexokinase (EC 2.7.1.1) is an enzyme that phosphorylates hexoses (six-carbon sugars), forming hexose phosphate. Hexokinase possesses the ability to transfer an inorganic phosphate group from ATP to a substrate. Numerous hexokinase proteins from various organisms have been cloned and expressed. In some embodiments, the hexokinase includes the sequence set forth in UniProtKB accession number P04806 from Saccharomyces cerevisiae (Sc) (incorporated herein by reference) as well as sequences that are at least 60%, 70%, 80%, 85%, 90%, 95%, 98%, 99% identical thereto and have hexokinase activity.

The glucose-6-phosphate is then converted to fructose-6-phosphate by phosphoglucoseisomerase (Pgi) (EC 5.3.1.9). Accordingly, in addition to the foregoing, the terms “phosphoglucoisomerase” or “Pgi” refer to proteins that are capable of catalyzing the formation of fructose-6-phosphate from glucose-6-phosphate, and which share at least about 40%, 45%, 50%, 55%, 60%, 65%, 70%, 75%, 80%, 85%, 90%, 95%, 96%, 97%, 98%, 99% or greater sequence identity to SEQ ID NO:31, or at least about 50%, 60%, 70%, 80%, 90%, 95%, 96%, 97%, 98%, 99% or greater sequence similarity, as calculated by NCBI BLAST, using default parameters and wherein the enzyme has phosphoglucoisomerase activity.

In another or further embodiment, a system or recombinant microorganism provided herein includes expression of a phosphofructokinase (Pfk, polyphosphate-dependent Pfk or homolog or variants thereof). In one embodiment, this expression is operable to be combined with other enzymes in the metabolic pathway. The Pfk is operable to be derived from G. stearothermophilus (SEQ ID NO:32). In another embodiment, an engineered variant of Pfk is operable to be used so long as it has phosphofructokinase activity and is operable to convert fructose-6-phosphate to fructose-1,6-bisphosphate. Such engineered variants are operable to be obtained by site-directed mutagenesis, directed evolutions and the like. Thus, included within the disclosure are polypeptides that are at least 85-99% identical to a sequence as set forth in SEQ ID NO:32 and having phosphofructokinase activity (see, e.g., SEQ ID nOs:33-34).

In addition to the foregoing, the terms “fructose 1,6 bisphosphate aldolase” or “Fba” refer to proteins that are capable of catalyzing the formation of dihydroxyacetone phosphate and glyceraldehyde-3-phosphate from fructose 1,6-bisphosphate, and which share at least about 40%, 45%, 50%, 55%, 60%, 65%, 70%, 75%, 80%, 85%, 90%, 95%, 96%, 97%, 98%, 99% or greater sequence identity, or at least about 50%, 60%, 70%, 80%, 90%, 95%, 96%, 97%, 98%, 99% or greater sequence similarity, as calculated by NCBI BLAST, using default parameters, to SEQ ID NO:35. Additional homologs include: Synechococcus elongatus PCC 6301 YP_170823.1 having 26% identity to SEQ ID NO:35; Vibrio nigripulchritudo ATCC 27043 ZP_08732298.1 having 80% identity to SEQ ID NO:35; Methylomicrobium album BG8 ZP_09865128.1 having 76% identity to SEQ ID NO:35; Pseudomonas fluorescens Pf0-1 YP 350990.1 having 25% identity to SEQ ID NO:35; and Methylobacterium nodulans ORS 2060 YP_002502325.1 having 24% identity to SEQ ID NO:35. Thus, the disclosure includes the use of polypeptides having from 26% to 100% identity to SEQ ID NO:35, wherein the polypeptide has bisophosphate aldolase activity. The sequences associated with the foregoing accession numbers are incorporated herein by reference.

In addition to the foregoing, the terms “triose phosphate isomerase” or “Tpi” refer to proteins that are capable of catalyzing the formation of glyceraldehyde-3-phosphate from dihydroxyacetone phosphate (DHAP), and which share at least about 40%, 45%, 50%, 55%, 60%, 65%, 70%, 75%, 80%, 85%, 90%, 95%, 96%, 97%, 98%, 99% or greater sequence identity, or at least about 50%, 60%, 70%, 80%, 90%, 95%, 96%, 97%, 98%, 99% or greater sequence similarity, as calculated by NCBI BLAST, using default parameters, to SEQ ID NO:36. Additional homologs include: Rattus norvegicus AAA42278.1 having 45% identity to SEQ ID NO:36; Homo sapiens AAH17917.1 having 45% identity to SEQ ID NO:36; Bacillus subtilis BEST7613 NP_391272.1 having 40% identity to SEQ ID NO:36; Synechococcus elongatus PCC 6301 YP_171000.1 having 40% identity to SEQ ID NO:36; and Salmonella enterica subsp. enterica serovar typhi str. AG3 ZP_06540375.1 having 98% identity to SEQ ID NO:36. Thus, the disclosure incudes the use of polypeptides that have from 40% to 100% identity to SEQ ID NO:36 and have triose phosphate isomerase activity. The sequences associated with the foregoing accession numbers are incorporated herein by reference.

In a further step of the pathway, glyceraldehyde-3-phosphate is operable to be converted to 1,3-bisphosphoglycerate. In one embodiment, this enzymatic step includes a “purge valve system” (as discussed elsewhere herein). For example, glyceraldehyde-3-phosphate dehydrogenase (Gap, Tdh) converts glyceraldehyde-3-phosphate to 1,3-bisphospho-glycerate. In one embodiment, a wild-type Gap is used that uses NAD⁺ as a cofactor (see, e.g., SEQ ID NO:37) or a mutant Gap comprising a P191D mutation (relative to the sequence of SEQ ID NO:37 and as shown in SEQ ID NO:38). In another embodiment, a mutant Gap (mGap; e.g., having a D34A/L35R/T35K mutation; relative to the sequence of SEQ ID NO:37 and as shown in SEQ ID NO:39) is used that uses NADP⁺ as a cofactor. In yet another embodiment, a combination of Gap and mGap (GapM6) are used. A molecular purge valve including a water generating NADH oxidase (NoxE) that specifically oxidizes NADH, but not NADPH, is operable be used to recycle (“purge”) NADH when a wild-type gap or P118D mutant gap is used that preferentially uses NAD⁺.

In addition to the foregoing, the terms “NADH oxidase” or “NoxE” refer to proteins that are capable of oxidizing NADH to NAD*, and which share at least about 40%, 45%, 50%, 55%, 60%, 65%, 70%, 75%, 80%, 85%, 90%, 95%, 96%, 97%, 98%, 99% or greater sequence identity, or at least about 50%, 60%, 70%, 80%, 90%, 95%, 96%, 97%, 98%, 99% or greater sequence similarity, as calculated by NCBI BLAST, using default parameters, to SEQ ID NO:18.

The pathway can further convert 1,3-bisphosphoglycerate to 3-phosphoglycerate by use of phosphoglycerate kinase (EC 2.7.2.3) (PGK; e.g., as provided in SEQ ID NO:40, or a homolog or variant thereof that is at least 80% identical thereto) which catalyzes the reversible transfer of a phosphate group from 1,3-bisphosphoglycerate (1,3-BPG) to ADP producing 3-phosphoglycerate (3-PG) and ATP. A molecular purge valve for ATP is operable to be present to recycle ADP using, for example, a GTPase or other enzyme or a homolog or variant thereof).

The 3-phosphoglycerate can then be converted by a phosphoglycerate mutase (pgm; e.g., as provided in SEQ ID NO:41, or a homolog or variant thereof that is at least 80% identical thereto) to 2-phosphoglycerate.

An enolase (eno; e.g., as provided in SEQ ID NO:42, or a homolog or variant thereof that is at least 80% identical thereto) is operable to then convert the 2-phosphoglycerate to phosphenolpyruvate (PEP).

A pyruvate kinase (pyk; e.g., as provided in SEQ ID nOs:43, 44, and 45, or a homolog or variant thereof that is at least 80% identical to any of SEQ ID NO:43, 44 or 45) converts PEP to pyruvate.

As mentioned above, pyruvate dehydrogenase (PDH) is inhibited by products of the pathway. Thus, a PDH Bypass is operable to be used to covert pyruvate to acetyl-coA. The PDH Bypass includes two enzymatic steps: (i) pyruvate→acetyl phosphate catalyzed by pyruvate oxidase (e.g., PyOx from Aerococcus viridans; EC 1.2.3.3; see SEQ ID NO:46); and (ii) acetyal phosphate→acetyl-coA catalyzed by an acetyl phosphate transferase (aka phosphate acetyltransferase) (e.g., PTA from G. stearothermophilus).

As used herein, a PyOx used in the composition and methods of the disclosure include sequences that are at least 85%, 90%, 95%, 98%, or 99% identical to SEQ ID NO:46 and have pyruvate oxidase activity.

Phosphate acetyltransferase (EC 2.3.1.8) is an enzyme that catalyzes the chemical reaction of acetyl-CoA+phosphate to CoA+acetyl phosphate and vice versa. Phosphate acetyltransferase is encoded in E. coli by pta. PTA is involved in conversion of acetate to acetyl-CoA. Specifically, PTA catalyzes the conversion of acetyl-coA to acetyl-phosphate. PTA homologs and variants are known. There are approximately 1075 bacterial phosphate acetyltransferases available on NCBI. For example, such homologs and variants include phosphate acetyltransferase Pta (Rickettsia felis URRWXCa12) gi|670040211|gb|AAY60947.11 (67004021); phosphate acetyltransferase (Buchnera aphidicola str. Cc (Cinara cedri)) gi|116256910|gb|ABJ90592.1|(116256910); pta (Buchnera aphidicola str. Cc (Cinara cedri)) gi|116515056|ref|YP_802685.1| (116515056); pta (Wigglesworthia glossinidia endosymbiont of Glossina brevipalpis) gi|25166135|dbj|BAC24326.1|(25166135); Pta (Pasteurella multocida subsp. multocida str. Pm70) gi|12720993|gb|AAK02789.1|(12720993); Pta (Rhodospirillum rubrum) gi|25989720|gb|AAN75024.11 (25989720); pta (Listeria welshimeri serovar 6b str. SLCC5334) gi|116742418|emb|CAK21542.1|(116742418); Pta (Mycobacterium avium subsp. paratuberculosis K-10) gi|41398816|gb|AAS06435.1|(41398816); phosphate acetyltransferase (pta) (Borrelia burgdorferi B31) gi|15594934|ref|NP_212723.1|(15594934); phosphate acetyltransferase (pta) (Borrelia burgdorferi B31) gi|2688508|gb|AAB91518.1|(2688508); phosphate acetyltransferase (pta) (Haemophilus influenzae Rd KW20) gi|1574131|gb|AAC22857.1|(1574131); Phosphate acetyltransferase Pta (Rickettsia bellii RML369-C) gi|91206026|ref|YP_538381.1|(91206026); Phosphate acetyltransferase Pta (Rickettsia bellii RML369-C) gi|91206025|ref|YP_538380.1|(91206025); phosphate acetyltransferase pta (Mycobacterium tuberculosis F11) gi|148720131|gb|ABR04756.1|(148720131); phosphate acetyltransferase pta (Mycobacterium tuberculosis str. Haarlem) gi|134148886|gb|EBA40931.11 (134148886); phosphate acetyltransferase pta (Mycobacterium tuberculosis C) gi|124599819|gb|EAY58829.1|(124599819); Phosphate acetyltransferase Pta (Rickettsia bellii RML369-C) gi|91069570|gb|ABE05292.1|(91069570); Phosphate acetyltransferase Pta (Rickettsia bellii RML369-C) gi|91069569|gb|ABE05291.1|(91069569); phosphate acetyltransferase (pta) (Treponema pallidum subsp. pallidum str. Nichols) gi|15639088|ref|NP_218534.1|(15639088); and phosphate acetyltransferase (pta) (Treponema pallidum subsp. pallidum str. Nichols) gi|3322356|gb|AAC65090.11 (3322356), each sequence associated with the accession number is incorporated herein by reference in its entirety.

Turning again to FIG. 2C, the pathway includes the conversion of acetyl-coA to acetoacetyl-coA. The conversion of acetyl-coA to acetoacetyl-CoA is performed by an acetyl-CoA acetyltransferase (e.g., PhaA). Numerous acetyl-coA acetyltransferases are known in the art. For example, acetyl-coA acetyltransferase from R. eutropha. In another embodiment, the acetyl-coA acetyl transferase has an amino acid sequence that is at least 85%, 90%, 95%, 98%, 99%, or 100% identical to SEQ ID NO:47.

Acetoacetyl-CoA and acetyl-Coa are operable to be converted to HMG-CoA by the enzyme HMG-CoA synthase having an A110G mutation (see, e.g., SEQ ID NO:48) or a homolog or variant thereof having at least 85%, 90%, 95%, 98%, or 99% (e.g., 85%-95%) sequence identity thereto.

The HMG-CoA is then reduced to mevalonate by the actions of NADPH and HMG-CoA reductase (see, e.g., SEQ ID NO:49) or a homolog or variant thereof having at least 85%, 90%, 95%, 98%, or 99% (e.g., 85%-95%) sequence identity thereto.

Mevalonate is then phosphorylated by ATP and the action of mevalonate kinase (MVK) to produce mevalonate-5-phosphate and ADP. Melavonate kinases are known in the art and include a sequence that is at least 85-100% (e.g., 85%, 90%, 95%, 98%, 99%) identical to the sequence of SEQ ID NO:50 and which have mevalonate kinase activity.

The mevalonate-5-phosphate is further phosphorylated by ATP and the actions of phosphomevalonate kinase (PMVK) to produce mevalonate-5-diphosphate and ADP. Phosphomevalonate kinases are known in the art and include a sequence that is at least 85-100% (e.g., 85%, 90%, 95%, 98%, 99%) identical to the sequence of SEQ ID NO:51 and which have phophomevalonate kinase activity.

Mevalonate-5-diphosphate is decarboxylated by ATP and the actions of diphosphomevalonate decarboxylase (MDC) to produce ADP, CO₂ and isopentyl pyrophosphate. Diphosphomevalonate decarboxylases are known in the art and include sequence that is at least 85-100% (e.g., 85%, 90%, 95%, 98%, 99%) identical to the sequence of SEQ ID NO:52 and which have diphosphomevalonate kinase activity.

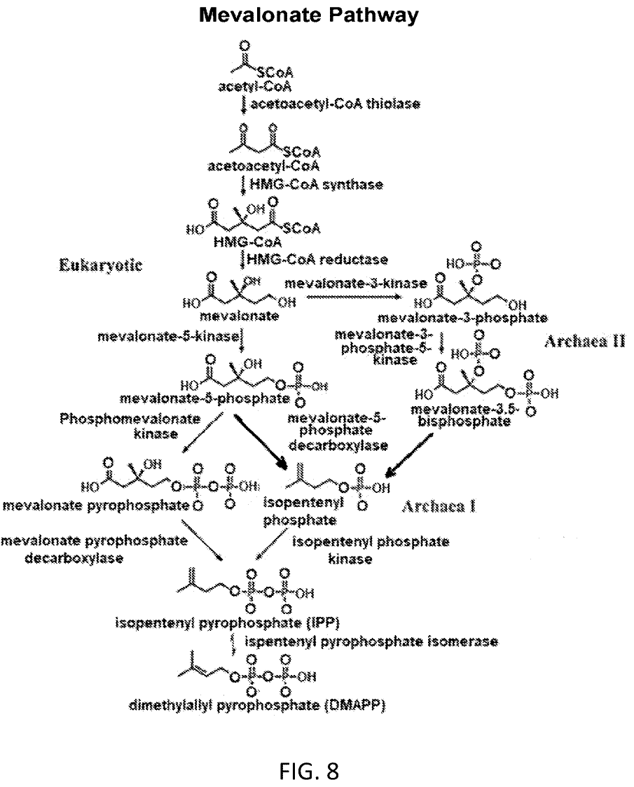

Various other mevalonate pathways are operable to be used (see, e.g., FIG. 8).

Geranyl pyrophosphate (GPP) is then formed from the combination of DMAPP and isopentyl pyrophosphate in the presence of farnesyl-PP synthase having an S82F mutation relative to SEQ ID NO:53. In one embodiment, the farnesyl-diphosphate synthase has a sequence that is at least 95%, 98%, 99%, or 100% identical to SEQ ID NO:53 having an S82F mutation and which is capable of forming geranyl pyrophosphate from DMAPP and isopentyl pyrophosphate.

GPP is then operable to be used as a substrate for a number of pathways leading to prenyl-flavinoids, geranyl-flavonoics, prenyl-stilbenoids, geranyl-stilbenoids, CBGA, CBGVA, CBDA, CBDVA, CBGVA, CBCVA, THCA, and THCVA (see, e.g., FIGS. 2A-2B)

For example, with the NphB mutant, as described above, in hand (e.g., an M23 mutant), the ability to produce CBGA directly from glucose and OA was tested using the full synthetic biochemistry system, including the PDH bypass (see, FIGS. 2A-2B and FIG. 3C). The initial productivity using M23 in the system was 67 mg L⁻¹ hr⁻¹ with a final titer of 744±34 mg L⁻¹ of CBGA. This was 100-fold faster than CBGA production using WT NphB, and reached a titer 21-fold higher. It is noted that with the mutant NphB enzyme, maximum titers were reached within 24 hours and the production stopped, yet with the wild-type enzyme, the system ran continuously for up to 4 days suggesting that enzymes and cofactors remain active and viable for longer periods of time. It was noted that once ˜500 mg L⁻¹ CBGA was produced, the reactions turned cloudy. The precipitate was collected and a mix of enzymes was identified in the precipitate by SDS-PAGE analysis, indicating that high-levels of CBGA in solution causes enzymes to precipitate. A more effective system was developed to remove product during the reaction.

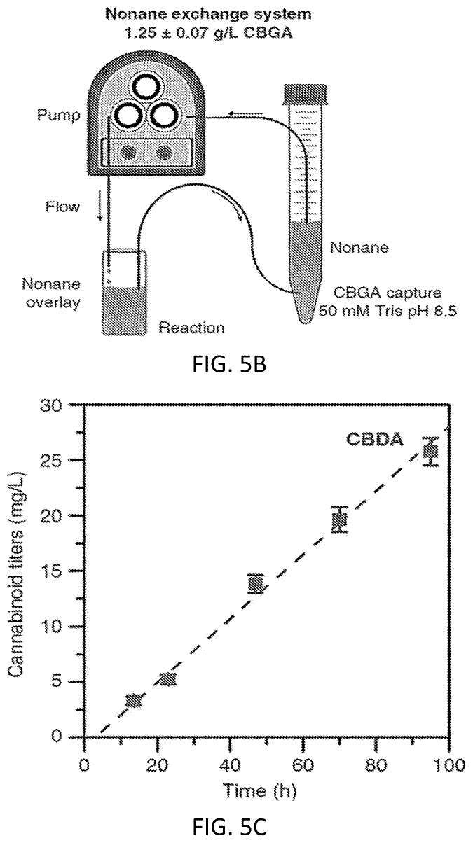

Although a nonane overlay was used in the reactions to extract CBGA, CBGA is more soluble in water than nonane, which limits the amount of CBGA that is operable to be extracted with a simple overlay. Thus, a flow system was designed that would capture CBGA from the nonane layer and trap it in a separate water reservoir (FIG. 5B). By implementing this flow system, a lower concentration of CBGA was maintained in the reaction vessel to mitigate enzyme precipitation. The flow system indeed improved the final titers to 1.2 g/L.

Experiments were then performed to produce the precursor of many rare cannabinoids, CBGVA, by replacing OA in the system with divirinic acid (DA) (see, e.g., FIG. 2C). The designed enzymes were first tested to determine if they would be active on a DA substrate. The two best mutants M23 and M31 were tested as well as WT NphB for their ability to produce CBGVA. The kinetic data shown in Table 2 indicated that M31 was far superior, with catalytic efficiencies 15-fold higher than M23 and 650-fold higher than WT NphB. Thus, further efforts utilized M31 to produce CBGVA from glucose and divarinic acid. As shown in FIG. 5A, CBGVA was produced at a max productivity of ˜107 mg L⁻¹ hr⁻¹, and reached a final titer of 1.74±0.09 g L⁻¹, converting 92% of the divarinic acid added to CBGVA. The nonane flow system was not needed for the production of CBGVA because CBGVA was less potent in precipitating enzymes.

To demonstrate that the approach is ultimately operable to be used to prepare additional cannabinoids, CBDA synthase was employed to convert CBGA into CBDA and CBGVA into CBDVA. For CBDA, the nonane overlay contained a significant quantity of CBGA, so by simply transferring the nonane overlay to a solution containing CBDA synthase, CBGA was converted into CBDA at a constant rate of 14.4±0.8 mg L⁻¹ hr⁻¹ mg⁻¹ total protein⁻¹ for 4 days.

Due to the limited solubility of CBGVA in nonane, the CBGVA was extracted and added to a reaction containing CBDA synthase. The product of the CBDA synthase was in fact CBDVA using GC-MS.

The disclosure thus provides a cell free system for the production of GPP. Further the disclosure provides a cell free approach for the production of an array of pure cannabinoids and other prenylated natural products using the GPP pathway in combination with a mutant NphB or using substrates for the mutant NphB of the disclosure. The success of this method uses the engineered prenyltransferase of the disclosure (e.g., NphB mutants as described above), which was active, highly specific and eliminated the need for the native transmembrane prenyltransferase. The modularity and flexibility of the synthetic biochemistry platform provided herein has the benefits of a bio-based approach, but removes the complexities of satisfying living systems. For example, GPP toxicity did not factor into the design process. Moreover, OA is not taken up by yeast so the approach of adding it exogenously would not necessarily be possible in cells. Indeed, the flexibility of cell free systems greatly facilitates the design-build-test cycles required for further optimization, additional pathway enzymes, and reagent and co-factor modifications.

Turning to the overall pathway of FIGS. 2A-2C, the disclosure provides a number of steps catalyzed by enzymes to covert a “substrate” to a product. In some instances, a step may utilize a co-factor, but some steps do not use co-factors (e.g., NAD(P)H, ATP/ADP, etc.). Table 3 provides a list of enzymes, organisms, and reaction amounts used as well as accession numbers (the sequences associated with such accession numbers are incorporated herein by reference).

TABLE 3

| | | | | Amount | | |
|---|---|---|---|---|---|---|
| | | | | Added | | |
| | Enzyme | | | to Rxn | | Acquisition |
| | Abb. | Full Name | Organism | (mg/mL) | | Number |
| 1 | Hex | Hexokinase | S. cerevisiae | 0.02 | | Sigma Aldrich |
| 2 | Pgi | Glucose-6-phosphate | G. thermodentrificans | 0.48 | | ABO6822 or |
| | | isomerase | | | | ARA98689.1 |
| 3 | PfkA | Phosphofructokinase | G. stearothermophilus | 0.18 | | KOR92562 or |
| | | | | | | P00512.2 |
| 4 | Fba | Fructose-1,6- | S. aureus | 0.03 | | BAR10119 or |
| | | bisphosphate aldolase | | | | PSN28048.1 |
| 5 | TpiA | Triose phosphate isomerase | G. stearothermophilus | 0.16 | | KOR95273 or |
| | | | | | | P00943.2 |
| 6 | Gap | Gald-3-P dehydrogenase | E. coli K12 | 0.07 | | NP_416293 |
| 7 | mGap | Gald-3-P dehydrogenase | G. stearothermophilus | 0.18 | | NP_416293 |
| | | D34A/L35R/T36K | | | | |
| 8 | NoxE | NADH Oxidase | L. lactis | 0.25 | | WP_015425842 |
| 9 | Pgk | Phosphoglycerate Kinase | G. stearothermophilus | 0.06 | | NP_415276 |
| 10 | dPgm | Phosphoglycerate | E. coli K12 | 0.29 | | NP_417259 |
| | | Mutase (2,3 BPG dependent) | | | | |
| 11 | Eno | Enolase | E. coli K12 | 0.08 | | KOR95272 or |
| | | | | | | BAE76853.1 |
| 12 | PykF | Pyruvate Kinase | E. coli K12 | 0.37 | | NP_416191 |
| | | (FBP dependent) | | | | |
| | PDH | Pyruvate Dehydrogenase | E. coli K12 | 0.99 | | |
| | | AceE | | | | NP_414656 |
| | | AceF | | | | NP_414657 |
| | | Lpd | | | | NP_414658 |
| 13 | PyOx | Pyruvate Oxidase | A. viridans | 1 | U | AG Scientific |
| 14 | PTA | Acetyl-phosphate | G. stearothermophilus | 0.06 | | WP_053532564 |
| | | transferase | | | | |
| 15 | PhaA | Acetyl-CoA acetyltransferase | R. eutropha | 0.12 | | CAJ92573 |
| 16 | HMGS | HMG-CoA Synthase | E. faecalis | 0.18 | | WP_010785222 |
| | A110G | A110G | | | | |
| 17 | HMGR | HMG-CoA Reductase | E. faecalis | 0.16 | | AAG02439 |
| 18 | MVK | Mevalonate Kinase | M. mazei | 0.14 | | AAM31458 |
| 19 | PMVK | Phosphomevalonate | S. pneumonia | 0.2 | | WP_000562411 |
| | | Kinase | | | | |
| 20 | MDC | Diphosphomevalonate | S. pneumonia | 0.19 | | NP_357933 |
| | | Kinase | | | | |
| 21 | IDI | Isopentenyl-PP Isomerase | E. coli K12 | 0.3 | | NP_417365 |
| 22 | FPPS | Farnesyl-PP synthase | G. stearothermophilus | 0.09 | | KOR95521 |
| | S82F | S82F | | | | |
| 23 | NphB | Aromatic prenyltransferase | Streptomyces sp. CL190 | Variable | | BAE00106.1 |
| 24B | CBDAS | Cannabidiolic Acid Synthase | C. sativa | | | AKC34419 |
| 25 | Ppase | Pyrophosphatase | G. stearothermophilus | 0.11 | | O05724 |
| 26 | Cat | Catalase | C. glutamicum | 0.1 | U | Sigma Aldrich |

| | | | | |
|---|---|---|---|---|
| GorA | Glutathione Reductase | E. coli K12 | 0.06 | NP_417957 |

As described above, prenylation of olivetolate by GPP is carried out by the activity of the mutant NphB polypeptides described herein and above.

In one embodiment, the pathway of the present invention utilizes polyphosphate kinase (PPK), malonyl-CoA synthetase (MatB), and pyrophosphatase (PPase) in the ATP regeneration pathway. In one embodiment, the polyphosphate kinase is MBP-AaPPK. In one embodiment, the pyrophosphatase is derived from G. stearothermophilus.

The disclosure provides an in vitro method of producing prenylated compounds and moreover, an in vitro method for producing cannabinoids and cannabinoid precursors (e.g., CBGA, CBGVA or CBGXA where ‘X’ refers to any chemical group). In one embodiment, cell-free preparations are operable to be made through, for example, three methods. In one embodiment, the enzymes of the pathway, as described herein, are purchased and mixed in a suitable buffer and a suitable substrate is added and incubated under conditions suitable for production of the prenylated compound, the cannabinoid, or the cannabinoid precursor. In some embodiments, the enzyme is operable to be bound to a support or expressed in a phage display or other surface expression system and, for example, fixed in a fluid pathway corresponding to points in the metabolic pathway's cycle.

FIG. 6A-B depict the pathway as various “modules” (e.g., glycolysis module, mevalonate/isoprenoid module, cannabinoid module, polyketide module). For example, the isoprenoid module produces the isoprenoid geranyl pyrophosphate (GPP) from acetyl-CoA via the mevalonate pathway. The aromatic polyketide module utilizes a Type III polyketide synthase (PKS) to convert hexanoyl-CoA and malonyl-CoA (derived from acetyl-CoA) into olivetolic acid (OA). The cannabinoid module uses products from the isoprenoid module and the polyketide module to yield cannabigerolic acid, which is then converted into the final cannabinoid by a cannabinoid synthase.

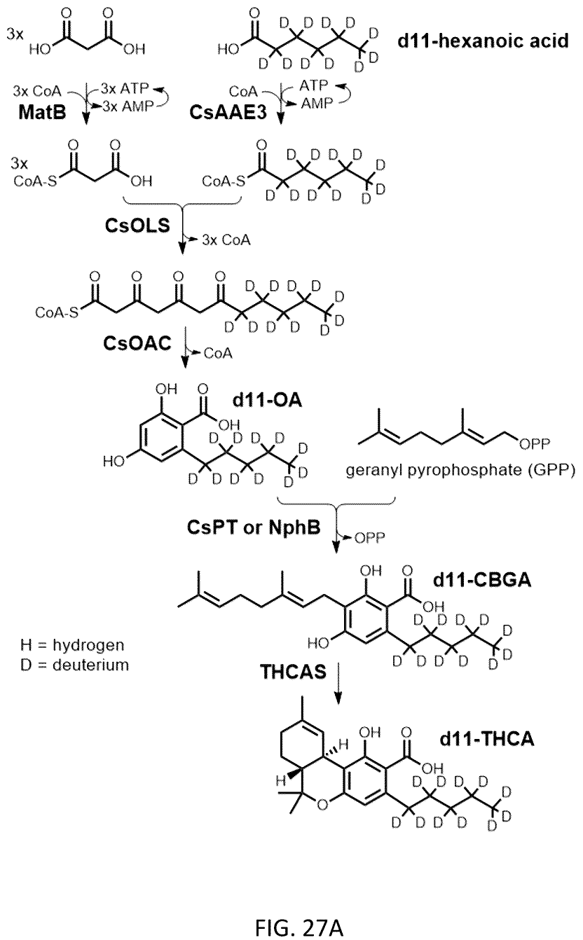

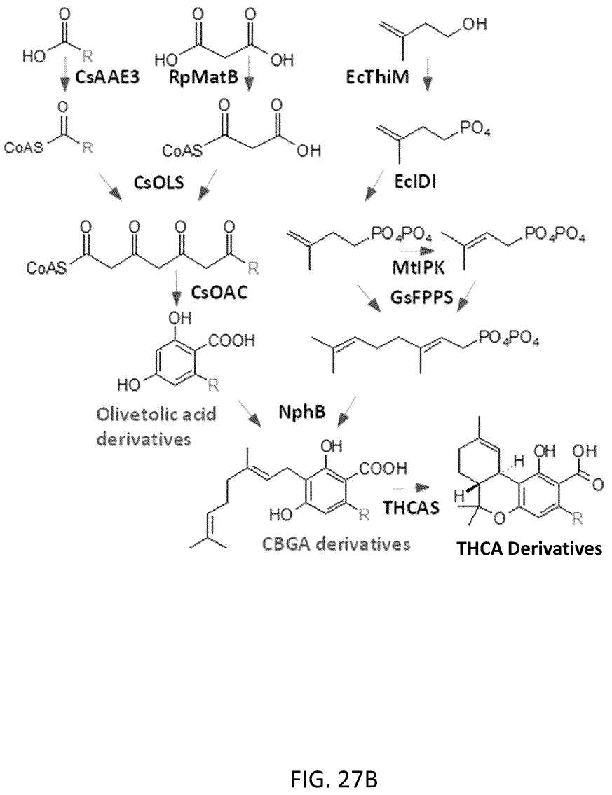

FIG. 27A illustrates one embodiment of a cell-free biosynthesis pathway for a deuterated cannabinoid. Unlabeled malonic acid and a deuterated fatty acid (e.g., fully deuterated, partially deuterated) is activated to a corresponding CoA thioester. The deuterated fatty acid d11-hexanoic acid is activated to d11-hexanoyl CoA in the presence of acyl-activating enzyme (AAE) 3, while the malonic acid is activated to malonyl CoA in the presence of malonyl-CoA synthetase. The CoA thioesters then undergo elongation and cyclization to produce d11-olivetolic acid (dl 1-OA). In the example shown in FIG. 27A, the deuterated fatty acid is dl 1-hexanoic acid. The present invention is compatible with alternative deuterated fatty acids. For example, and not limitation, if d7-butryic acid is the deuterated fatty acid, d7-divarinic acid (d7-DA) is produced using the same method. As a result, the activation of deuterated fatty acids to the corresponding CoA thioester for incorporation into or late-stage modification of small molecules is operable to be a general approach.

Following formation of dl 1-OA, prenylation is operable to occur through the action of CsPT or engineered NphB enzyme in the presence of geranyl pyrophosphate (GPP) to produce the deuterated cannabinoid d11-cannabigerolic acid (d11-CBGA). d11-CBGA is then operable to be cyclized through the action of tetrahydrocannabidiolic acid synthase (THCAS) to form d11-tetrahydrocannabidiolic acid (d11-THCA). Alternative deuterated cannabinoids are operable to be synthesized using alternative cannabinoid synthases (e.g., CBDAS, CBCAS). One of ordinary skill in the art will appreciate that the use of cannabidiolic acid synthase (CBDAS) results in the production of a deuterated cannabinoid in the form of cannabidiolic acid (CBDA) (e.g., d11-CBDA) rather than tetrahydrocannabidiolic acid. Similarly, the use of cannabichromenic acid synthase (CBCAS) results in the production of a deuterated cannabinoid in the form of cannabichromenic acid (CBCA) (e.g., d11-CBCA).

FIG. 27B illustrates one embodiment of a cell-free biosynthesis pathway for a deuterated cannabinoid, including the pathway for deriving geranyl pyrophosphatase (GPP), according to one embodiment of the present invention. Hydroxyethylthiazole kinase (ThiM) is used to produce isopentenyl phosphate from isoporenol. In the presence of isopentenyl diphosphate isomerase, the isopentenyl phosphate is converted into isopentenyl diphosphate. Isopentenyl diphosphate is operable to be converted to dimethylallyl pyrophosphate (DMAPP). Both isopentenyl diphosphate and DMAPP are operable to be converted into geranyl polyphosphate in the presence of farnesyl pyrophosphate synthase (FPPS). Geranyl polyphosphate is then operable for use in the cell-free biosynthesis pathway as described herein. One of ordinary skill in the art will appreciate that this process is additionally detailed in FIG. 7, though it is shown here to illustrate the step of the cell-free biosynthesis pathway at which the geranyl polyphosphate is utilized upon production.

In one embodiment, upon formation of the THCA compound of the present invention, the compound is purified to isolate the produced THCA as disclosed herein. Upon purification of the THCA, the THCA is operable to be decarboxylated and purified in order to obtain THC.

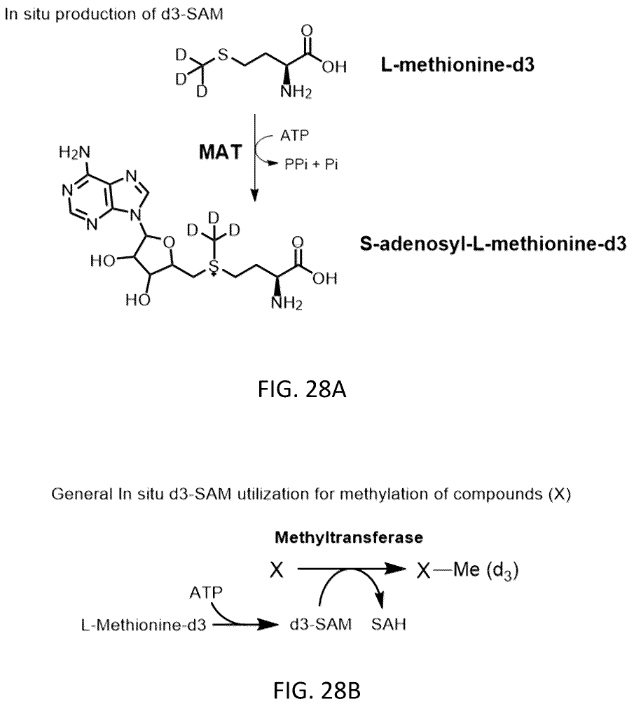

FIG. 28A illustrates one embodiment of a deuterated methyl compound. Deuterated d3-S-adenosyl-methionine (d3-SAM) is operable to be produced from d3-L-methionine and ATP through the action of SAM Synthetase (MAT). As shown in FIG. 28B, d3-SAM is operable to be used by methyltransferases to methylate various small molecules.

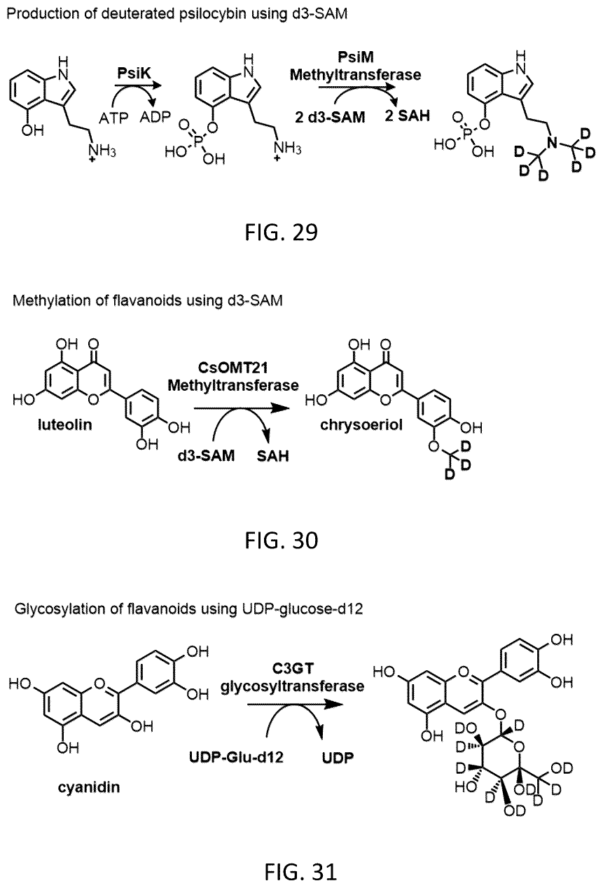

FIG. 29 illustrates an example of deuterated methylated psilocybin using a specific methyltransferase (e.g., PsiM methyltransferase).

FIG. 30 illustrates an example of deuterated methylated chrysoeriol using a specific methyltransferase (e.g., CsOMT21 methyltransferase).

FIG. 31 illustrates an example of the synthesis of a deuterated glucoside produced using UDP-glucose-d12.

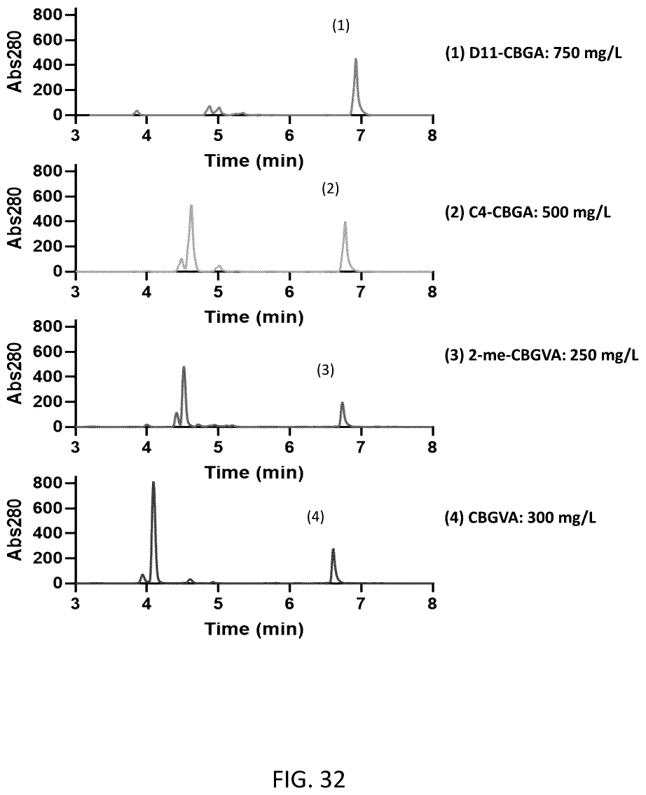

FIG. 32 illustrates an HPLC (high-performance liquid chromatography) trace of cell-free production of deuterated (1), even-chain (2), branched (3), and rare (4) cannabinoids (e.g., CBG derivatives).

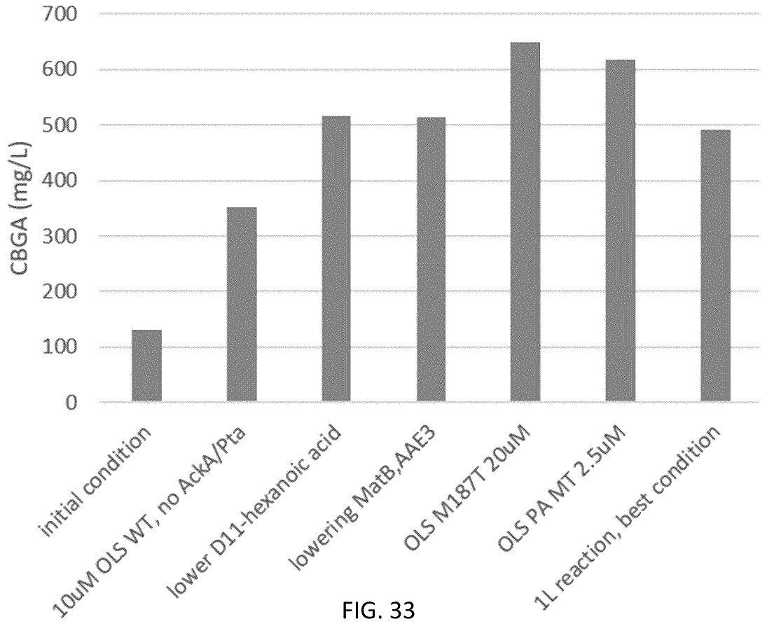

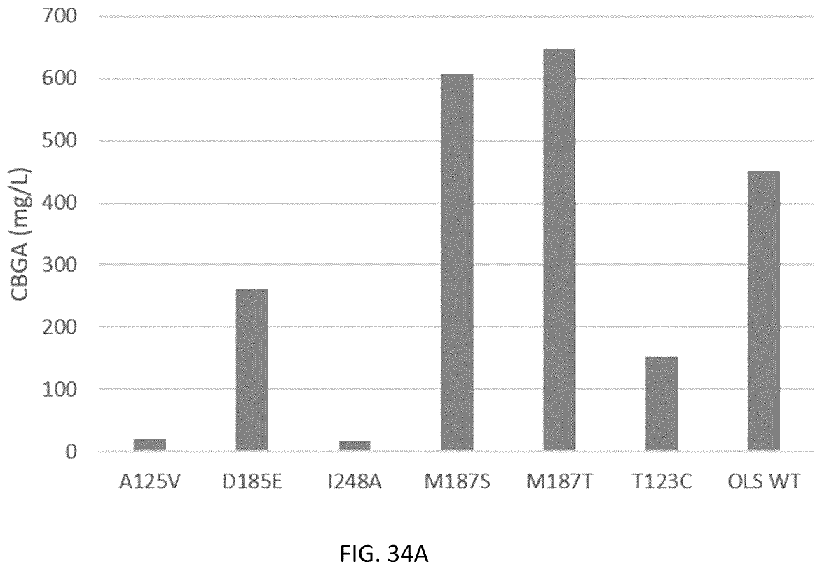

FIG. 33 illustrates a bar graph indicating a summary of the progress made during pathway optimization according to one embodiment of the present invention. All reactions were completed at 200 uL scale except for the 1 L scaled reaction indicated by the farthest bar to the right of the graph, labeled thus.

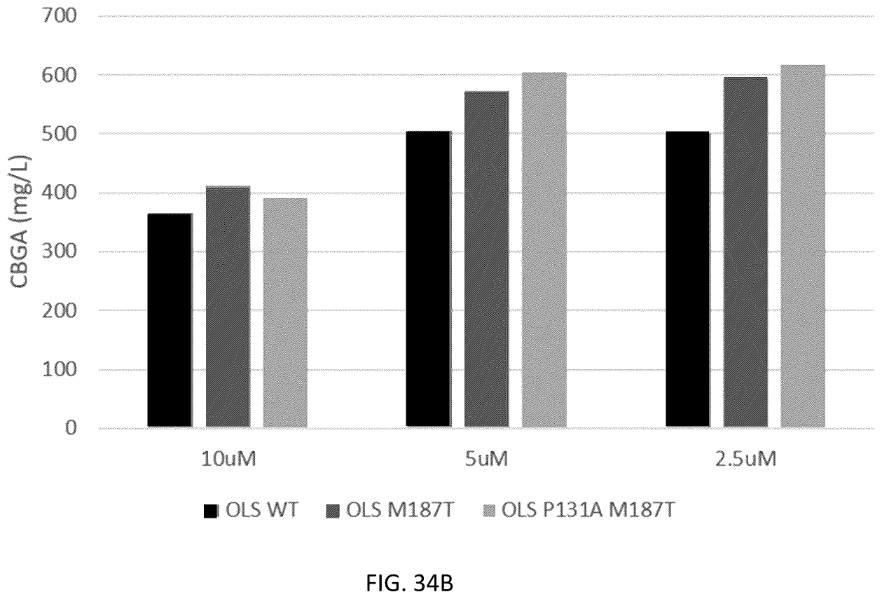

FIGS. 34A-34B illustrate CBGA production of OLS variants. Each variant was screened to determine the production level of CBGA from the OLS variant. The OLS variants were observed at varying enzyme loads to determine the corresponding level of CBGA production.

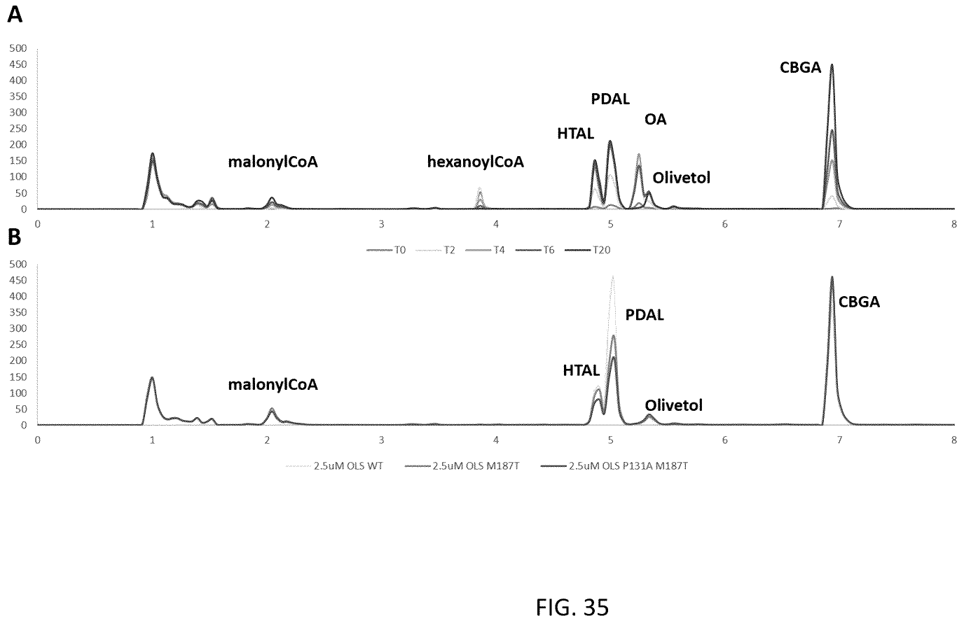

FIG. 35 illustrates a time course of CBGA synthesis reaction according to one embodiment of the present invention. The time course of CBGA synthesis reaction using OLS M187T is depicted in graph A, with timepoints at 0 h, 2 h, 4 h, 6 h and 20 h. A slight build up of olivetolic acid can be observed after 4 h, showing that SimplePath or NphB may be limiting in the reaction. Graph B depicts 20 h traces of OLS variants showing difference in side product formation between WT, M187T and P131A M187T.

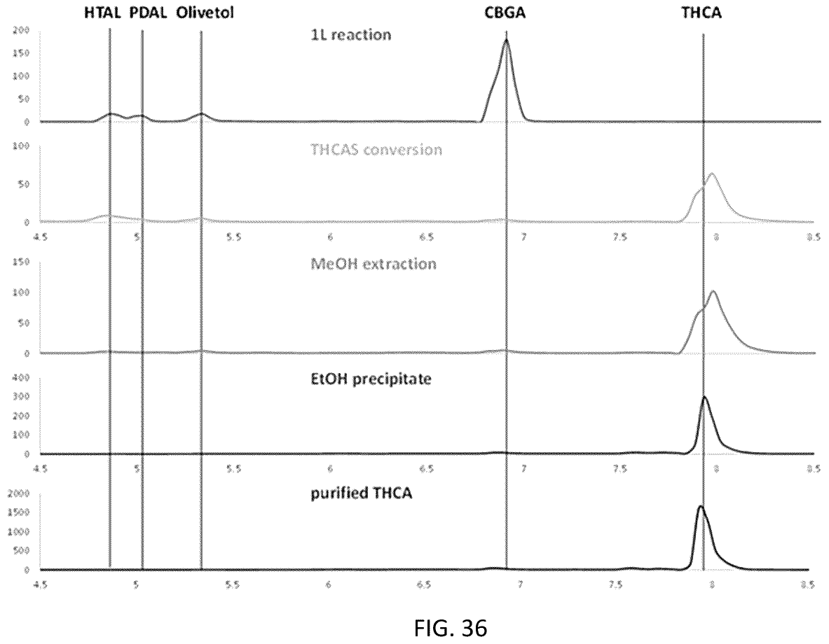

FIG. 36 illustrates an HPLC trace of CBGA and THCA activity according to one embodiment of the present invention. The HPLC analysis of 1 L CBGA reaction, conversion to THCA using P. pastoris THCAS supernatant, methanol extraction of THCA reaction, ethanol precipitation of methanol extraction, and purified THCA trace. There is a significant difference in ABS at 216 nm with increasing purity to visualize/quantify impurities.

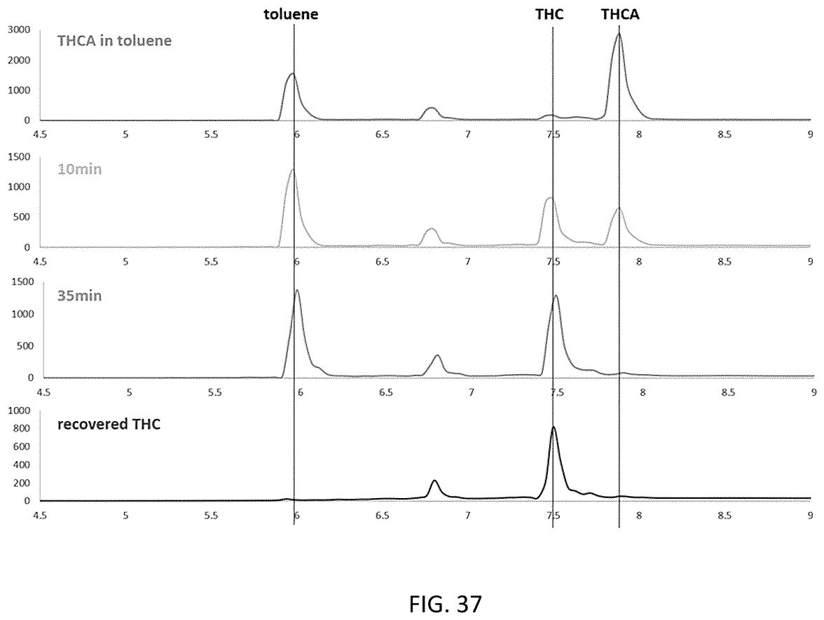

FIG. 37 illustrates an HPLC trace of THC in toluene according to one embodiment of the present invention. The HPLC traces of THCA decarboxylation reaction in toluene at 95° C. The starting material in toluene is shown at the top. The reaction after ten minutes is displayed by the HPLC below that. The reaction after thirty-five minutes is displayed below the ten minute reaction, and of the recovered THC product is indicated by the lower most HPLC line.

One of ordinary skill in the art will appreciate that alternative starting materials and enzymes are operable to produce alternative products. The present invention is not limited to the examples provided herein.

In another embodiment, one or more polynucleotides encoding one or more enzymes of the pathway are cloned into one or more microorganism under conditions whereby the enzymes are expressed. Subsequently the cells are lysed and the lysed preparation including the one or more enzymes derived from the cell are combined with a suitable buffer and substrate (and one or more additional enzymes of the pathway, if necessary) to produce the prenylated compound, the cannabinoid, or the cannabinoid precursor. Alternatively, the enzymes are operable to be isolated from the lysed preparations and then recombined in an appropriate buffer. In yet another embodiment, a combination of purchased enzymes and expressed enzymes are used to provide a pathway in an appropriate buffer. In one embodiment, heat stabilized polypeptide/enzymes of the pathway are cloned and expressed. In one embodiment, the enzymes of the pathway are derived from thermophilic microorganisms. The microorganisms are then lysed, the preparation heated to a temperature wherein the heat stabilized polypeptides of the pathway are active and other polypeptides (not of interest) are denatured and become inactive. The preparation thereby includes a subset of all enzymes in the microorganism and includes active heat-stable enzymes. The preparation is then operable to be used to carry out the pathway to produce the prenylated compound, the cannabinoid, or the cannabinoid precursor.

For example, to construct an in vitro system, all the enzymes are operable to be acquired commercially or purified by affinity chromatography, tested for activity, and mixed together in a properly selected reaction buffer.

An in vivo system is also contemplated using all or portions of the foregoing enzymes in a biosynthetic pathway engineered into a microorganism to obtain a recombinant microorganism.

The disclosure also provides recombinant organisms including metabolically engineered biosynthetic pathways that include a mutant nphB for the production of prenylated compounds and optionally further includes one or more additional organisms expressing enzymes for the production of cannabinoids (e.g., a co-culture of one set of microorganism expressing a partial pathway and a second set of microorganism expression yet a further or final portion of the pathway etc.).

In one embodiment, the disclosure provides a recombinant microorganism including elevated expression of at least one target enzyme as compared to a parental microorganism or encodes an enzyme not found in the parental organism. In another or further embodiment, the microorganism provides a reduction, disruption, or knockout of at least one gene encoding an enzyme that competes with a metabolite necessary for the production of a desired metabolite or which produces an unwanted product. In one embodiment, the recombinant microorganism expresses an enzyme that produces at least one metabolite involved in a biosynthetic pathway for the production of, for example, the prenylated compound or the cannabinoids or cannabinoid precursor. In general, the recombinant microorganism includes at least one recombinant metabolic pathway that includes a target enzyme and optionally further includes a reduction in activity or expression of an enzyme in a competitive biosynthetic pathway. The pathway acts to modify a substrate or metabolic intermediate in the production of, for example, a prenylated compound, a cannabinoid, or a cannabinoid precursor. The target enzyme is encoded by, and expressed from, a polynucleotide derived from a suitable biological source. In some embodiments, the polynucleotide includes a gene derived from a bacterial or yeast source and is recombinantly engineered into the microorganism of the disclosure. In another embodiment, the polynucleotide encoding the desired target enzyme is naturally occurring in the organism but is recombinantly engineered to be overexpressed compared to the naturally expression levels.

The term “microorganism” includes prokaryotic and eukaryotic microbial species from the Domains Archaea, Bacteria, and Eucarya, the latter including yeast and filamentous fungi, protozoa, algae, or higher Protista. The terms “microbial cells” and “microbes” are used interchangeably with the term microorganism.

The term “prokaryotes” is art recognized and refers to cells which contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

“Bacteria”, or “eubacteria”, refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; and (11) Thermotoga and Thermosipho thermophiles.

“Gram-negative bacteria” include cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium.

“Gram positive bacteria” include cocci, nonsporulating rods, and sporulating rods. The genera of gram positive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

As used herein, an “activity” of an enzyme is a measure of its ability to catalyze a reaction resulting in a metabolite, i.e., to “function”, and is operable to be expressed as the rate at which the metabolite of the reaction is produced. For example, enzyme activity is operable to be represented as the amount of metabolite produced per unit of time or per unit of enzyme (e.g., concentration or weight), or in terms of affinity or dissociation constants.

The term “biosynthetic pathway”, also referred to as “metabolic pathway”, refers to a set of anabolic or catabolic biochemical reactions for converting (transmuting) one chemical species into another (see, e.g., FIGS. 2A-2C). Gene products belong to the same “metabolic pathway” if they, in parallel or in series, act on the same substrate, produce the same product, or act on or produce a metabolic intermediate (i.e., metabolite) between the same substrate and metabolite end product. The disclosure provides recombinant microorganism having a metabolically engineered pathway for the production of a desired product or intermediate.

Accordingly, metabolically “engineered” or “modified” microorganisms are produced via the introduction of genetic material into a host or parental microorganism of choice thereby modifying or altering the cellular physiology and biochemistry of the microorganism. Through the introduction of genetic material, the parental microorganism acquires new properties, e.g., the ability to produce a new, or greater quantities of, an intracellular metabolite or to express a polypeptide nor normally expressed. In an illustrative embodiment, the introduction of genetic material into a parental microorganism results in a new or modified ability to produce acetyl-phosphate and/or acetyl-CoA through a PDH bypass using pyruvate oxidase and acetylphosphate transferase. The genetic material introduced into the parental microorganism contains gene(s), or parts of gene(s), coding for one or more of the enzymes involved in a biosynthetic pathway for the production of prenylated compounds or cannabinoids or cannabinoid precursors, and is also operable to include additional elements for the expression and/or regulation of expression of these genes, e.g., promoter sequences.

An engineered or modified microorganism is also operable to include in the alternative or in addition to the introduction of a genetic material into a host or parental microorganism, the disruption, deletion, or knocking out of a gene or polynucleotide to alter the cellular physiology and biochemistry of the microorganism. Through the reduction, disruption, or knocking out of a gene or polynucleotide, the microorganism acquires new or improved properties (e.g., the ability to produce a new or greater quantities of an intracellular metabolite, improve the flux of a metabolite down a desired pathway, and/or reduce the production of undesirable by-products) or eliminates the enzyme from cell free preparations that are operable to compete with a biosynthetic pathway developed from lysed preparations.

An “enzyme” means any substance, typically composed wholly or largely of amino acids making up a protein or polypeptide that catalyzes or promotes, more or less specifically, one or more chemical or biochemical reactions.

A “protein” or “polypeptide”, which terms are used interchangeably herein, includes one or more chains of chemical building blocks called amino acids that are linked together by chemical bonds called peptide bonds. A protein or polypeptide is operable to function as an enzyme.

As used herein, the term “metabolically engineered” or “metabolic engineering” involves rational pathway design and assembly of biosynthetic genes, genes associated with operons, and control elements of such polynucleotides, for the production of a desired metabolite, such as an acetyl-phosphate and/or acetyl-CoA, higher alcohols or other chemical, in a microorganism. “Metabolically engineered” is operable to further include optimization of metabolic flux by regulation and optimization of transcription, translation, protein stability and protein functionality using genetic engineering and appropriate culture condition including the reduction of, disruption, or knocking out of, a competing metabolic pathway that competes with an intermediate leading to a desired pathway. A biosynthetic gene is operable to be heterologous to the host microorganism, either by virtue of being foreign to the host, or being modified by mutagenesis, recombination, and/or association with a heterologous expression control sequence in an endogenous host cell. In one embodiment, where the polynucleotide is xenogenetic to the host organism, the polynucleotide is operable to be codon optimized.

A “metabolite” refers to any substance produced by metabolism or a substance necessary for or taking part in a particular metabolic process that gives rise to a desired metabolite, chemical, alcohol, or ketone. A metabolite is operable to be an organic compound that is a starting material (e.g., glucose etc.), an intermediate in (e.g., acetyl-coA), or an end product (e.g., CBDA) of metabolism. Metabolites are operable to be used to construct more complex molecules, or they are operable to be broken down into simpler ones. Intermediate metabolites are operable to be synthesized from other metabolites, perhaps used to make more complex substances, or broken down into simpler compounds, often with the release of chemical energy.

A “mutation” means any process or mechanism resulting in a mutant protein, enzyme, polynucleotide, gene, or cell. This includes any mutation in which a protein, enzyme, polynucleotide, or gene sequence is altered, and any detectable change in a cell arising from such a mutation. Typically, a mutation occurs in a polynucleotide or gene sequence, by point mutations, deletions, or insertions of single or multiple nucleotide residues. A mutation includes polynucleotide alterations arising within a protein-encoding region of a gene as well as alterations in regions outside of a protein-encoding sequence, such as, but not limited to, regulatory or promoter sequences. A mutation in a gene is operable to be “silent”, i.e., not reflected in an amino acid alteration upon expression, leading to a “sequence-conservative” variant of the gene. This generally arises when one amino acid corresponds to more than one codon. A mutation that gives rise to a different primary sequence of a protein is operable to be referred to as a mutant protein or protein variant.

A “native” or “wild-type” protein, enzyme, polynucleotide, gene, or cell, means a protein, enzyme, polynucleotide, gene, or cell that occurs in nature.

A “parental microorganism” refers to a cell used to generate a recombinant microorganism. The term “parental microorganism” describes, in one embodiment, a cell that occurs in nature, i.e., a “wild-type” cell that has not been genetically modified. The term “parental microorganism” further describes a cell that serves as the “parent” for further engineering. In this latter embodiment, the cell is operable to have been genetically engineered, but serves as a source for further genetic engineering.

For example, a wild-type microorganism is operable to be genetically modified to express or over express a first target enzyme such as a hexokinase. This microorganism is operable to act as a parental microorganism in the generation of a microorganism modified to express or over-express a second target enzyme, e.g., a fructose-1,6-bisphosphate aldolase. In turn, that microorganism is operable be modified to express or over express e.g., an NADH oxidase and a Gald-3-phosphate dehydrogenase (and mutants thereof), which is operable be further modified to express or over express a third target enzyme, e.g., a phosphoglycerate kinase etc. As used herein, “express” or “over express” refers to the phenotypic expression of a desired gene product. In one embodiment, a naturally occurring gene in the organism is operable to be engineered such that it is linked to a heterologous promoter or regulatory domain, wherein the regulatory domain causes expression of the gene, thereby modifying its normal expression relative to the wild-type organism. Alternatively, the organism is operable to be engineered to remove or reduce a repressor function on the gene, thereby modifying its expression. In yet another embodiment, a cassette including the gene sequence operably linked to a desired expression control/regulatory element is engineered in to the microorganism.

Accordingly, a parental microorganism functions as a reference cell for successive genetic modification events. Each modification event is operable to be accomplished by introducing one or more nucleic acid molecules into the reference cell. The introduction facilitates the expression or over-expression of one or more target enzyme or the reduction or elimination of one or more target enzymes. It is understood that the term “facilitates” encompasses the activation of endogenous polynucleotides encoding a target enzyme through genetic modification of e.g., a promoter sequence in a parental microorganism. It is further understood that the term “facilitates” encompasses the introduction of exogenous polynucleotides encoding a target enzyme into a parental microorganism.

Polynucleotides that encode enzymes useful for generating metabolites including homologs, variants, fragments, related fusion proteins, or functional equivalents thereof, are used in recombinant nucleic acid molecules that direct the expression of such polypeptides in appropriate host cells, such as bacterial or yeast cells. The sequences provided herein and the accession numbers provide those of skill in the art the ability to obtain and obtain coding sequences for various enzymes of the disclosure using readily available software and basis biology knowledge.

The sequence listing appended hereto provide exemplary polypeptides useful in the methods described herein. It is understood that the addition of sequences which do not alter the activity of a polypeptide molecule, such as the addition of a non-functional or non-coding sequence (e.g., polyHIS tags), is a conservative variation of the basic molecule.

It is understood that a polynucleotide described herein include “genes” and that the nucleic acid molecules described above include “vectors” or “plasmids.”

The term “polynucleotide,” “nucleic acid” or “recombinant nucleic acid” refers to polynucleotides such as deoxyribonucleic acid (DNA), and, where appropriate, ribonucleic acid (RNA).

The term “expression” with respect to a gene or polynucleotide refers to transcription of the gene or polynucleotide and, as appropriate, translation of the resulting mRNA transcript to a protein or polypeptide. Thus, as will be clear from the context, expression of a protein or polypeptide results from transcription and translation of the open reading frame.

Those of skill in the art will recognize that, due to the degenerate nature of the genetic code, a variety of codons differing in their nucleotide sequences are operable to be used to encode a given amino acid. A particular polynucleotide or gene sequence encoding a biosynthetic enzyme or polypeptide described above are referenced herein merely to illustrate an embodiment of the disclosure, and the disclosure includes polynucleotides of any sequence that encode a polypeptide comprising the same amino acid sequence of the polypeptides and proteins of the enzymes utilized in the methods of the disclosure. In similar fashion, a polypeptide is operable to typically tolerate one or more amino acid substitutions, deletions, and insertions in its amino acid sequence without loss or significant loss of a desired activity. The disclosure includes such polypeptides with alternate amino acid sequences, and the amino acid sequences encoded by the DNA sequences shown herein merely illustrate exemplary embodiments of the disclosure.

The disclosure provides polynucleotides in the form of recombinant DNA expression vectors or plasmids, as described in more detail elsewhere herein, that encode one or more target enzymes. Generally, such vectors can either replicate in the cytoplasm of the host microorganism or integrate into the chromosomal DNA of the host microorganism. In either case, the vector is operable to be a stable vector (i.e., the vector remains present over many cell divisions, even if only with selective pressure) or a transient vector (i.e., the vector is gradually lost by host microorganisms with increasing numbers of cell divisions). The disclosure provides DNA molecules in isolated (i.e., not pure, but existing in a preparation in an abundance and/or concentration not found in nature) and purified (i.e., substantially free of contaminating materials or substantially free of materials with which the corresponding DNA would be found in nature) form.

A polynucleotide of the disclosure is operable to be amplified using cDNA, mRNA, or alternatively, genomic DNA, as a template and appropriate oligonucleotide primers according to standard PCR amplification techniques and those procedures described in the Examples section below. The nucleic acid so amplified is operable to be cloned into an appropriate vector and characterized by DNA sequence analysis. Furthermore, oligonucleotides corresponding to nucleotide sequences are operable to be prepared by standard synthetic techniques, e.g., using an automated DNA synthesizer.

The disclosure provides a number of polypeptide sequences in the sequence listing accompanying the present application, which are operable to be used to design, synthesize and/or isolate polynucleotide sequences using the degeneracy of the genetic code or using publicly available databases to search for the coding sequences.

It is also understood that an isolated polynucleotide molecule encoding a polypeptide homologous to the enzymes described herein are operable to be created by introducing one or more nucleotide substitutions, additions, or deletions into the nucleotide sequence encoding the particular polypeptide, such that one or more amino acid substitutions, additions, or deletions are introduced into the encoded protein. Mutations can be introduced into the polynucleotide by standard techniques, such as site-directed mutagenesis and PCR-mediated mutagenesis. In contrast to those positions where it is desirable to make a non-conservative amino acid substitution, in some positions it is preferable to make conservative amino acid substitutions.

As will be understood by those of skill in the art, it is operable to be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons are operable to be substituted to reflect the preferred codon usage of the host, a process sometimes called “codon optimization” or “controlling for species codon bias.”

Optimized coding sequences containing codons preferred by a particular prokaryotic or eukaryotic host (see also, Murray et al. (1989) Nucl. Acids Res. 17:477-508, which is incorporated herein by reference in its entirety) are operable to be prepared, for example, to increase the rate of translation or to produce recombinant RNA transcripts having desirable properties, such as a longer half-life, as compared with transcripts produced from a non-optimized sequence. Translation stop codons are also operable to be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al. (1996) Nucl. Acids Res. 24: 216-218, which is incorporated herein by reference in its entirety). Methodology for optimizing a nucleotide sequence for expression in a plant is provided, for example, in U.S. Pat. No. 6,015,891, and the references cited therein. U.S. Pat. No. 6,015,891 is incorporated herein by reference in its entirety.

The term “substrate” or “suitable substrate” refers to any substance or compound that is converted or meant to be converted into another compound by the action of an enzyme. The term includes not only a single compound, but also combinations of compounds, such as solutions, mixtures and other materials which contain at least one substrate, or derivatives thereof. Further, the term “substrate” encompasses not only compounds that provide a starting material, but also intermediate and end product metabolites used in a pathway associated with a metabolically engineered microorganism as described herein.

“Transformation” refers to the process by which a vector is introduced into a host cell. Transformation (or transduction, or transfection) is operable to be achieved by any one of a number of means including electroporation, microinjection, biolistics (or particle bombardment-mediated delivery), or Agrobacterium mediated transformation.

A “vector” generally refers to a polynucleotide that is operable to be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes,” that is, that replicate autonomously or are operable to integrate into a chromosome of a host cell. A vector is also operable to be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not episomal in nature, or it is operable to be an organism which includes one or more of the above polynucleotide constructs such as an Agrobacterium or a bacterium.

The various components of an expression vector are operable to vary widely, depending on the intended use of the vector and the host cell(s) in which the vector is intended to replicate or drive expression. Expression vector components suitable for the expression of genes and maintenance of vectors in E. coli, yeast, Streptomyces, and other commonly used cells are widely known and commercially available. For example, suitable promoters for inclusion in the expression vectors of the disclosure include those that function in eukaryotic or prokaryotic host microorganisms. Promoters are operable to comprise regulatory sequences that allow for regulation of expression relative to the growth of the host microorganism or that cause the expression of a gene to be turned on or off in response to a chemical or physical stimulus. For E. coli and certain other bacterial host cells, promoters derived from genes for biosynthetic enzymes, antibiotic-resistance conferring enzymes, and phage proteins are operable to be used and include, for example, the galactose, lactose (lac), maltose, tryptophan (trp), beta-lactamase (bla), bacteriophage lambda PL, and T5 promoters. In addition, synthetic promoters, such as the tac promoter (U.S. Pat. No. 4,551,433, which is incorporated herein by reference in its entirety), are also operable to be used. For E. coli expression vectors, it is useful to include an E. coli origin of replication, such as from pUC, plP, pl, or pBR.

Thus, recombinant expression vectors contain at least one expression system, which, in turn, is composed of at least a portion of a gene coding sequences operably linked to a promoter and optionally termination sequences that operate to effect expression of the coding sequence in compatible host cells. The host cells are modified by transformation with the recombinant DNA expression vectors of the disclosure to contain the expression system sequences either as extrachromosomal elements or integrated into the chromosome.

In addition, and as mentioned above, homologs of enzymes useful for generating metabolites are encompassed by the microorganisms and methods provided herein. The term “homologs” used with respect to an original enzyme or gene of a first family or species refers to distinct enzymes or genes of a second family or species which are determined by functional, structural or genomic analyses to be an enzyme or gene of the second family or species which corresponds to the original enzyme or gene of the first family or species. Most often, homologs will have functional, structural or genomic similarities. Techniques are known by which homologs of an enzyme or gene are operable to readily be cloned using genetic probes and PCR. Identity of cloned sequences as homolog are operable to be confirmed using functional assays and/or by genomic mapping of the genes.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences).

As used herein, two proteins (or a region of the proteins) are substantially homologous when the amino acid sequences have at least about 30%, 40%, 50% 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences are operable to be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology is operable to be adjusted upwards to correct for the conservative nature of the substitution. Means for making this adjustment are well known to those of skill in the art (see, e.g., Pearson et al., 1994, hereby incorporated herein by reference).

In some instances, “isozymes” are operable to be used that carry out the same functional conversion/reaction, but which are so dissimilar in structure that they are typically determined to not be “homologous”.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is also operable to be referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using measure of homology assigned to various substitutions, deletions, and other modifications, including conservative amino acid substitutions. For instance, GCG contains programs such as “Gap” and “Bestfit” which are operable to be used with default parameters to determine sequence homology or sequence identity between closely related polypeptides, such as homologous polypeptides from different species of organisms or between a wild type protein and a mutein thereof. See, e.g., GCG Version 6.1.

A typical algorithm used to compare a molecule sequence to a database containing a large number of sequences from different organisms is the computer program BLAST (Altschul, 1990; Gish, 1993; Madden, 1996; Altschul, 1997; Zhang, 1997), especially blastp or tblastn (Altschul, 1997). Typical parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

When searching a database containing sequences from a large number of different organisms, it is typical to compare amino acid sequences. Database searching using amino acid sequences is operable to be measured by algorithms other than BLASTp known in the art. For instance, polypeptide sequences are operable to be compared using FASTA, a program in GCG Version 6.1. FASTA provides alignments and percent sequence identity of the regions of the best overlap between the query and search sequences (Pearson, 1990, hereby incorporated herein by reference). For example, percent sequence identity between amino acid sequences is operable to be determined using FASTA with its default parameters (a word size of 2 and the PAM250 scoring matrix), as provided in GCG Version 6.1, which is hereby incorporated herein by reference in its entirety.

The disclosure provides accession numbers and sequences for various genes, homologs and variants useful in the generation of recombinant microorganism and proteins for use in in vitro systems. It is to be understood that homologs and variants described herein are exemplary and non-limiting. Additional homologs, variants and sequences are available to those of skill in the art using various databases including, for example, the National Center for Biotechnology Information (NCBI) access to which is available on the World-Wide-Web.

It is well within the level of skill in the art to utilize the sequences and accession number described herein to identify homologs and isozymes that are operable to be used or substituted for any of the polypeptides used herein. In fact, a BLAST search of any one of the sequences provide herein will identify a plurality of related homologs.

Culture conditions suitable for the growth and maintenance of a recombinant microorganism provided herein are known (see, e.g., “Culture of Animal Cells-A Manual of Basic Technique” by Freshney, Wiley-Liss, N.Y. (1994), Third Edition). The skilled artisan will recognize that such conditions are operable to be modified to accommodate the requirements of each microorganism.

It is understood that a range of microorganisms are operable to be modified to include all or part of a recombinant metabolic pathway suitable for the production of prenylated compounds, cannabinoids, or cannabinoid precursors. It is also understood that various microorganisms are operable to act as “sources” for genetic material encoding target enzymes suitable for use in a recombinant microorganism provided herein.

As previously discussed, general texts which describe molecular biological techniques useful herein, including the use of vectors, promoters and many other relevant topics, include Berger and Kimmel, Guide to Molecular Cloning Techniques, Methods in Enzymology Volume 152, (Academic Press, Inc., San Diego, Calif.) (“Berger”); Sambrook et al., Molecular Cloning-A Laboratory Manual, 2d ed., Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between greene Publishing Associates, Inc. and John Wiley & Sons, Inc., (supplemented through 1999) (“Ausubel”), each of which is incorporated herein by reference in its entirety.

Examples of protocols sufficient to direct persons of skill through in vitro amplification methods, including the polymerase chain reaction (PCR), the ligase chain reaction (LCR), Qp-replicase amplification and other RNA polymerase mediated techniques (e.g., NASBA), e.g., for the production of the homologous nucleic acids of the disclosure are found in Berger, Sambrook, and Ausubel, as well as in Mullis et al. (1987) U.S. Pat. No. 4,683,202; Innis et al., eds. (1990) PCR Protocols: A Guide to Methods and applications (Academic Press Inc. San Diego, Calif.) (“Innis”); Arnheim & Levinson (Oct. 1, 1990) C&EN 36-47; The Journal Of NIH Research (1991) 3: 81-94; Kwoh et al. (1989) Proc. Natl. Acad. Sci. USA 86: 1173; Guatelli et al. (1990) Proc. Nat'l. Acad. Sci. USA 87: 1874; Lomell et al. (1989) J. Clin. Chem 35: 1826; Landegren et al. (1988) Science 241: 1077-1080; Van Brunt (1990) Biotechnology 8: 291-294; Wu and Wallace (1989) Gene 4:560; Barringer et al. (1990) Gene 89:117; and Sooknanan and Malek (1995) Biotechnology 13:563-564, each of which is incorporated herein by reference in its entirety.

Improved methods for cloning in vitro amplified nucleic acids are described in Wallace et al., U.S. Pat. No. 5,426,039, which is incorporated herein by reference in its entirety.

Improved methods for amplifying large nucleic acids by PCR are summarized in Cheng et al. (1994) Nature 369: 684-685 and the references cited therein, in which PCR amplicons of up to 40 kb are generated. Cheng et al. (1994) Nature 369: 684-685 is incorporated herein by reference in its entirety. One of skill will appreciate that essentially any RNA can be converted into a double stranded DNA suitable for restriction digestion, PCR expansion and sequencing using reverse transcriptase and a polymerase. See, e.g., Ausubel, Sambrook and Berger, all supra.

The invention is illustrated in the following examples, which are provided by way of illustration and are not intended to be limiting.

### EXAMPLES

Chemicals and Reagents. Yeast hexokinase and Corynebacterium glutamicum catalase were purchased from Sigma Aldrich. Aerococcus viridians pyruvate oxidase was purchased from A.G. scientific. All cofactors and reagents were purchased from either Sigma Aldrich or Thermo Fisher Scientific, with the exception of olivetolic acid, which was purchased from Santa Cruz Biotechnology and divarinic acid, which was purchased from Toronto Research Chemicals.

Cloning and purification of enzymes. The NphB gene was purchased as a gene block from IDT DNA, and cloned into a pET 28(+) vector using the Gibson Assembly method. The remaining enzymes were amplified from genomic DNA or a plasmid, and cloned into pET28(+) using the same Gibson assembly method. All plasmids were transformed into BL21(DE3) Gold, and enzymes expressed in LB media with 50 μg/mL kanamycin. 1 μL cultures were inoculated with 2 mL of a saturated culture in the same media, and grown to an OD₆₀₀ of 0.5-0.8 at 37° C. The cultures were induced with 1 mM IPTG, and expressed at 18° C. for 16 hours. The cells were harvested by centrifugation at 2,500×g, and resuspended in ^{˜}20 mL lysis buffer: 50 mM Tris [pH 8.0], 150 mM NaCl, and 10 mM imidazole. The cells were lysed using an Emulsiflex instrument. The lysate was clarified by centrifugation at 20,000×g, and the supernatant was batch bound to 1 mL NiNTA resin for 30 mins at 4° C. The resin was transferred to a gravity flow column. The resin was washed with 10 column volumes of wash buffer: 50 mM Tris [pH 8.0], 150 mM NaCl, and 10 mM imidazole. The protein was then eluted with 2 column volumes of elution buffer: 50 mM Tris [pH 8.0], 150 mM NaCl, 250 mM imidazole and 30% (v/v) glycerol. Enzymes were flash frozen in elution buffer using liquid N₂, and the enzyme stocks were stored at −80° C.

PDH Cell-free Reactions. The PDH reactions were assembled in two parts. First the co-factors and substrates were combined in one tube, and the enzymes were combined in another. The reactions were initiated by mixing the co-factors and enzymes in a final volume of 200 μL. The final substrate and co-factor concentrations were as follows: 500 mM glucose, 1 mM 1,6 fructose bisphosphate, 4 mM ATP, 0.5 mM 2,3 bisphosphoglycerate, 0.5 mM NAD*, 1.5 mM CoA, 1.5 mM NADP⁺, 0.5 mM TPP, 6 mM MgCl₂, 10 mM KCl, 50 mM Tris [pH 8.0] and 20 mM phosphate buffer [pH 8.0], 5 mM glutathione and 0.5-5 mM 1,6 DHN. The reactions were quenched at 24 hours.

PDH Activity Assays. PDH was assayed for activity in the presence of several aromatic polyketides. The vehicle control was 1% ethanol, and the activity was compared to an assay without the aromatic polyketides. The final reaction volume was 200 μL, and contained 2 mM NADY, 2 mM CoA, 1 mM TPP, 5 mM MgCl₂, 5 mM KCl, 50 mM Tris pH 8.0, and 5 μL of 1.25 mg/mL PDH. The reactions were set up in a 96-well plate. The aromatic polyketides were added to a final concentration of 1 mM and the ethanol control was added to a final concentration of 1% (v/v). The plate was incubated at room temperature for 10 minutes, and the reactions were initiated with 10 μL of 100 mM pyruvate. The absorbance at 340 nm was monitored for 10 minutes using an M200 spectrometer. Because the aromatic molecules had a background absorbance at 340 nm, the reactions were blanked using the reaction mixture and aromatic molecule, but instead of initiating the reaction with pyruvate, water was added. The initial rates were determined using the initial slope of a linear fit. The amount of NADH produced per unit time was calculated using Beer's law, and the extinction coefficient of 6.22×103 M⁻¹ cm⁻¹. Reactions were performed in triplicate, and the average value and standard error were calculated.

PyOx/PTA Cell-free Reactions. The PyOx/PTA reactions were assembled in two pieces. First the co-factors and substrates were combined in one tube, and the enzymes were combined in another. The final co-factor and substrate concentrations in the 200 μL reaction were as follows: 500 mM glucose, 1 mM 1,6 fructose bisphosphate, 4 mM ATP, 0.5 mM 2,3 bisphosphoglycerate, 0.5 mM NAD*, 1.5 mM CoA, 3 mM mM NADP⁺, 0.5 mM TPP, 6 mM MgCl₂, 10 mM KCl, 50 mM Tris pH 8.0, and 50 mM phosphate buffer [pH 8.0]. The amount of enzyme added to each reaction is detailed in Table 3. The co-factors and enzymes were mixed to initiate the reaction, and a 500 μL nonane overlay was added to the top. The reactions were incubated at room temperature shaking gently on a gel shaker.

For 1,6 DHN/5-p-1,6 DHN: When the aromatic substrate was the varied component 0.5 to 5 mM of the aromatic substrate was added to the reaction, and the reactions were quenched at 24 hours. When time was the varied component, 5 mM of 1,6 DHN was added, and separate reactions were quenched at ˜12, 24, 48 and 72 hours.

For olivetolate/CBGA: The optimization of the cannabinoid pathway showed that the same titers are operable to be achieved with less glucose, so the glucose concentration was reduced to 150 mM. Additionally, increasing the NADP⁺ concentration to 6 mM and decreasing the ATP concentration to 1 mM led to higher titers of CBGA. The olivetolate concentration was set at 5 mM. The amount of NphB added to the reaction was variable. The data shown in FIG. 3C utilized 1.5 mg/mL NphB, and the reactions were quenched at ˜4, 8, 14, 24, 48, 72, and 96 hours. The data shown in FIG. 5A was achieved with 0.5 mg/mL of WT NphB and M23, and reactions were quenched at ˜6, 9, 12, 24, 48, 72, and 96 hours.

For divarinic acid/CBGVA: The conditions were very similar to the general method above except 150 mM glucose, 1 mM ATP, and 6 mM NADP⁺ was used and the reactions were quenched at ˜6, 9, 12, 24, and 48 hours. Additionally, the final concentration of the prenyl-transferase was 1 mg/mL, and AtaPT, NovQ, and NphB was tested with apigenin, daidzein, genistein, naringenin, and resveratrol. NphB was also tested with olivetol, olivetolate, and 1,6 DHN. The reactions were quenched at 24 h.

Quenching reactions. To quench the reactions, the aqueous and organic layer were transferred to a 1.5 mL microcentrifuge tube. The reaction vial was washed with 200 PL of ethyl acetate, which was then pooled with the reaction in the microcentrifuge tube. The samples were vortexed for 5-10 seconds and then centrifuged for 3 minutes at 13,000 rpm. The organic layer was removed, and the remaining aqueous layer was extracted 2 additional times with 200 μL of ethyl acetate. For each sample the organic extract was pooled, and then evaporated using a vacuum centrifuge. The samples were re-dissolved in methanol for HPLC analysis.

For olivetolate/CBGA: Due to the observed protein precipitation, the CBGA reactions shown in FIG. 5A were extracted in the presence of 0.12 g of urea (solid), to facilitate the extraction of CBGA. This was unnecessary for the WT NphB CBGA data in FIG. 3C because the proteins did not precipitate.

Quantification of products. The reactions were fractionated by reverse phase chromatography on a C18 column (4.6×100 mm) using a Thermo Ultimate 3000 HPLC. The column compartment temperature was set to 40° C., and the flow rate was 1 mL/min. The compounds were separated using a gradient elution with water+0.1% TFA (solvent A) and acetonitrile+0.1% TFA (solvent B) as the mobile phase. Solvent B was held at 20% for the first min. Then solvent B was increased to 95% B over 4 min, and 95% B was then held for 3 min. The column was then re-equilibrated to 20% B for three min, for a total run time of 11 min.

The cannabinoids (CBGA, CBDA, and CBDVA) were quantified using an external calibration curve derived from an analytical standard purchased from Sigma Aldrich. The 5-p-1,6-DHN and CBGVA nuclear magnetic resonance (NMR) samples were used to generate an external calibration curve because authentic standards were not available. A known concentration of the standard was dissolved in water, and then extracted using the method detailed above.

Quantify prenyl-products without authentic standards. Due to the lack of authentic standards for the prenyl-products prenyl-apigenin, prenyl-daidzein, prenyl-naringenin, prenyl-genistein, prenyl-resveratrol, and prenyl-olivetol, the prenyl-products were quantified based on substrate consumption. To generate a standard curve, serial dilutions of each aromatic substrate were subjected to the reaction mix, but to prevent product formation the prenyl-transferase was left out. Liquid chromatography-mass spectrometry was used to quantify the amount of substrate consumed by the reaction compared to the standard curve.

Electrospray ionization time-of-flight measurements were carried out on a Waters LCT-Premier XE Time of Flight Instrument controlled by MassLynx 4.1 software (Waters Corporation, Milford, Mass.). The instrument was equipped with the Multi Mode Ionization source operated in the electrospray mode. A solution of Leucine Enkephalin (Sigma Chemical, L9133) was used in the Lock-Spray to obtain accurate mass measurements. Samples were infused using direct loop injection on a Waters Acquity UPLC system. Samples were separated on a Waters Acquity UPLC system using an Acquity BEH C18 1.7 m column (50×2.1 mm) and were eluted with a gradient of 30-95% solvent B over 10 min (solvent A: water, solvent B: acetonitrile, both with 0.2% formic acid (vol/vol)). Mass spectra were recorded from a mass of 300-2000 Da.

NMR Spectroscopy. NMR spectroscopy was used to identify prenyl-products, and quantify 5-p-1,6-DHN.

For 1,6 DHN/5-p-1,6 DHN: The PyOx/PTA cell-free system was used to produce prenyl-DHN. 200 μL reactions were pooled, and extracted 3 times with an equivalent amount of nonane and then the nonane was evaporated. The product of the reactions was suspended in 500 L of deuterated methanol (CD₃OD), with 2 mM 1,3,5-trimethoxybenzene (TMB) as an internal standard. Spectra were collected on an AV400 Bruker NMR spectrometer. The amount of the prenylated compound in the sample was determined with reference to the internal TMB standard. The proton signal from TMB (3H, s) at 6.05 ppm were compared with an aromatic proton corresponding to 5-p-1,6-DHN (1H, d) at 7.27 ppm.

For divarinic acid/CBGVA: NMR was also used to identify the product of the enzymatic system with divarinic acid as the aromatic substrate. The PyOx/PTA system was set up as detailed above, and the reactions were quenched at 24 hours. The reactions were extracted as detailed above, and analyzed on the HPLC. There was a new major peak at 6.7 minutes that was predicted to be the prenylated divarinic acid. The HPLC peak was purified, removed the solvent, and re-dissolved the pure component in 600 μL of CD₃OD. A proton spectrum collected with an AV500 Bruker NMR spectrometer was compared to a proton spectrum published by Shoyama et al. for CBGVA to confirm that CBGVA was the main product. Based on the paper by Shoyama et al and the paper by Bohlman et al., it was concluded that the prenylation of divarinic acid occurs at the C3 carbon of divarinic acid.

Rosetta Design to modify the binding pocket of NphB to accept olivetolate. Olivetolate was placed in the active site of NphB in six different starting positions denoted as Olivetolate P1-6 in Table 4. ROSETTA was run 5 times for each olivetolate position for a total of 30 designs. The mutations predicted in each design are listed in Table 4. For each olivetolate position a consensus set of mutations (i.e., the most frequently chosen residue) was chosen to evaluate further: Consensus Group A through F (Table 4). The relative importance of each ROSSETTA suggested mutation was then evaluated. For each Consensus Group, the mutations were set back to WT residue, one at a time, and used ROSETTA to calculate the change in energy score (see Table 5). Those that caused the largest change in energy were deemed to be the most important mutants to include in the library for experimental testing.

TABLE 4

| | | | | | | Consensus | | | | | | Consensus |
|---|---|---|---|---|---|---|---|---|---|---|---|---|
| | Olivetolate P1 | | | | | Mutations | Olivetolate P2 | | | | | Mutations |
| Prediction # | 1 | 2 | 3 | 4 | 5 | Group A | 6 | 7 | 8 | 9 | 10 | Group B |
| V49 | I | T | I | I | I | I | T | S | S | S | T | S |
| M162 | | | | | | | | | | | | |
| F213 | N | N | N | N | N | N | N | N | N | N | N | N |
| A232 | N | N | N | N | S | N | S | S | S | S | S | S |
| I234 | T | T | T | T | T | T | T | T | T | T | T | T |
| V271 | N | H | N | N | N | N | N | N | N | N | N | N |
| G286 | | | | | | | | S | S | S | | S |
| Y288 | A | D | A | A | H | A | N | N | N | N | N | N |
| L298 | I | I | I | I | I | I | R | R | R | R | R | R |
| Energy Score | | | | | | −404 | | | | | | −410 |
| | | | | | | Consensus | | | | | | Consensus |
| | Olivetolate P3 | | | | | Mutations | Olivetolate P4 | | | | | Mutations |
| Prediction # | 11 | 12 | 13 | 14 | 15 | Group C | 16 | 17 | 18 | 19 | 20 | Group D |
| V49 | N | | N | N | S | N | T | N | T | N | T | T |
| M162 | C | C | CC | | C | C | | | | | | |
| F213 | N | N | N | N | N | N | G | G | G | G | G | G |
| A232 | | | | | | | | | | | | |
| I234 | T | T | T | T | T | T | T | T | T | T | T | T |
| V271 | N | N | N | N | N | N | H | N | H | N | H | H |
| G286 | S | | S | S | S | S | | | | | | |
| Y288 | S | A | S | S | N | S | D | S | N | S | D | N |
| L298 | R | R | R | R | R | R | I | I | I | I | I | I |
| Energy Score | | | | | | −405 | | | | | | −402 |
| | | | | | | Consensus | | | | | | Consensus |
| | Olivetolate P5 | | | | | Mutations | Olivetolate P6 | | | | | Mutations |
| Prediction # | 21 | 22 | 23 | 24 | 25 | Group E | 26 | 27 | 28 | 29 | 30 | Group F |
| V49 | S | S | I | S | S | S | G | G | G | S | S | G |
| M162 | R | R | R | R | R | R | R | R | R | R | R | R |
| F213 | N | N | N | N | N | N | N | N | N | N | N | N |
| A232 | N | S | N | S | N | S | S | S | S | S | S | S |
| I234 | T | T | T | T | T | T | T | T | T | T | T | T |
| V271 | N | N | S | N | N | N | A | N | N | A | A | N |
| G286 | | | | | | | | | | | | |
| Y288 | N | N | N | N | N | N | N | N | N | A | A | N |
| L298 | I | A | N | A | N | A | G | V | V | G | G | V |
| Energy Score | | | | | | −403 | | | | | | −398 |

TABLE 5

| Olivetolate Position 1 | | | | | Olivetolate Position 2 | | | | |
|---|---|---|---|---|---|---|---|---|---|
| | | | Energy | | | | | Energy | |
| Amino | Consensus | | Score of | | Amino | Consensus | | Score of | |
| Acid | Mutations | | Mutant −> | Energy | Acid | Mutations | | Mutant −> | Energy |
| Position | Group A | WT | WT | Difference | Position | Group B | WT | WT | Difference |
| 49 | I | V | −403 | 1 | 49 | S | V | −394 | 16 |
| 213 | N | F | −391 | 13 | 213 | N | F | −402 | 8 |
| 232 | N | A | −401 | 3 | 232 | S | A | −409 | 1 |
| 234 | T | I | −382 | 22 | 234 | T | I | −404 | 6 |
| 271 | N | V | −395 | 9 | 271 | N | V | −397 | 13 |
| 288 | A | Y | −392 | 12 | 296 | S | G | −409 | 1 |
| 298 | I | L | −404 | 0 | 288 | N | Y | −401 | 9 |
| | | | | | 298 | R | L | −408 | 2 |
| Mutation with largest effect | | | | | Mutation with largest effect | | | | |
| I234T | | | | | V49S | | | | |
| F213N | | | | | V271N | | | | |
| Y288A | | | | | Y288N | | | | |
| Olivetolate Position 3 | | | | | Olivetolate Position 4 | | | | |
| | | | Energy | | | | | Energy | |
| Amino | Consensus | | Score of | | Amino | Consensus | | Score of | |
| Acid | Mutations | | Mutant −> | Energy | Acid | Mutations | | Mutant −> | Energy |
| Position | Group C | WT | WT | Difference | Position | Group D | WT | WT | Difference |
| 49 | N | V | −391 | 14 | 49 | T | V | −401 | 1 |
| 162 | C | M | −404 | 1 | 213 | G | F | −98 | 304 |
| 213 | N | F | −390 | 15 | 234 | T | I | −372 | 30 |
| 234 | T | I | −400 | 5 | 271 | H | V | −398 | 4 |
| 271 | N | V | −396 | 9 | 288 | N | Y | −381 | 21 |
| 286 | S | G | −404 | 1 | 298 | I | L | −401 | 1 |
| 288 | S | Y | −394 | 11 | | | | | |
| 298 | R | L | −403 | 2 | | | | | |
| Mutation with largest effect | | | | | Mutation with largest effect | | | | |
| F213N | | | | | F213G | | | | |
| V49N | | | | | I234T | | | | |
| Y288S | | | | | Y288N | | | | |
| Olivetolate Position 5 | | | | | Olivetolate Position 6 | | | | |
| | | | Energy | | | | | Energy | |
| Amino | Consensus | | Score of | | Amino | Consensus | | Score of | |
| Acid | Mutations | | Mutant −> | Energy | Acid | Mutations | | Mutant −> | Energy |
| Position | Group E | WT | WT | Difference | Position | Group F | WT | WT | Difference |
| 49 | S | V | −398 | 5 | 49 | G | V | −383 | 15 |
| 162 | R | M | −402 | 1 | 162 | R | M | −398 | 0 |
| 213 | N | F | −318 | 85 | 213 | N | F | −388 | 10 |
| 232 | S | A | −327 | 76 | 232 | S | A | −388 | 10 |
| 234 | T | I | −398 | 5 | 234 | T | I | −388 | 10 |
| 271 | N | V | −391 | 12 | 271 | N | V | −390 | 8 |
| 288 | N | Y | −390 | 13 | 288 | N | Y | −367 | 31 |
| 298 | A | L | −394 | 9 | 298 | V | L | −397 | 1 |

| | |
|---|---|
| Mutations with largest effect | Mutations with largest effect |
| F213N | Y288N |
| A232S | V49G |
| Y288N | |

Initial NphB mutant library screening. For screening of the initial library, small scale expression and purifications were performed. 25 mL of LB media was inoculated with 25 μL of a saturated culture of BL21 DE3 Gold harboring the NphB expression plasmid. The cultures were incubated at 37° C. until the OD₆₀₀ reached 0.4-0.6. The expression of the NphB constructs were induced with the addition of 1 mM IPTG, followed by incubation for 18 hours at 18° C. Cells were harvested by centrifugation at 2500×g. The pellets were re-suspended in 500 μL of lysis buffer: 50 mM [Tris pH 8.0], 150 mM NaCl, and 5 mM imidazole and lysed by sonication. The cell lysate was clarified by centrifugation at 20,000×g for 10 minutes at 4° C., and the supernatant was incubated at 4° C. with 50 PL of NiNTA resin. A 96-well spin column plate was used to purify the NphB constructs. The supernatant/resin was applied to the column and centrifuged for 2 mins at 500×g. 500 μL of lysis buffer was then added, and the plate was centrifuged again for 1 minute at 500×g. The protein was eluted using 200 μL of elution buffer (50 mM Tris [pH 8.0], 150 mM NaCl, 250 mM imidazole, and 30% (v/v) glycerol).

The enzymes were assayed under the following conditions: 2.5 mM geranyl pyrophosphate, 5 mM olivetolate, 5 mM MgCl₂, 50 mM Tris pH 8.0, ˜0.1 mg/mL NphB mutant in a final volume of 100 μL. All enzymes were first diluted to 0.5 mg/mL using elution buffer so the final concentration of imidazole was the same in each reaction. The reactions were incubated for 12 hours at room temperature, then extracted 3 times with 100 μL of ethyl acetate. The organic extract was pooled for each reaction and the solvent was removed using a vacuum centrifuge. The samples were redissolved in 100 μL of methanol and subjected to HPLC analysis.

Focused NphB mutant library screening. For the focused library, 1 μL scale expression and purification of the NphB constructs as described above was performed. The enzymes were assayed under the following conditions: 2.5 mM GPP, 5 mM olivetolate, 5 mM MgCl₂, 50 mM Tris pH 8.0, and ˜1 mg/mL of NphB enzyme in a final volume of 100 μL. The reactions were incubated at room temperature for 1 hour. 40 μL of each reaction was quenched in 80 μL of acetonitrile. The samples were centrifuged for 5 minutes at 13,000 rpm to remove precipitated proteins. The supernatant was analyzed using HPLC as described above.

Enzyme Kinetic Parameters. The reactions were set up under the following conditions: 50 mM Tris [pH 8.0], 2.5 mM GPP, 5 mM MgCl₂, ˜27 μM enzyme, and olivetolate or divarinic acid was varied from 0.1 mM to 6 mM in a final volume of 200 μL. 40 μL of the reaction was quenched in 80 μl acetonitrile+0.1% TFA at the time intervals detailed below. The reactions were centrifuged for 5 minutes at 13,000-16,060×g to pellet the protein, and the supernatant was analyzed using the HPLC method detailed above. The initial rate was plotted vs the concentration of substrate, and fit with the Michaelis-Menten equation to determine the kinetic parameters k_{cat} and K_{M} (OriginPro). Each Michaelis-Menten curve was performed in triplicate. The average and standard deviation of the kinetic parameters are reported.

For olivetolate/CBGA: For WT, M1, M10 and M30, the time course was 3, 6, 9, and 12 minutes. For mutant 25, the reactions were quenched at 1, 2, 4, and 8 minutes, and for M31 the reactions were quenched at 1, 2, 4, and 6 minutes.

For divarinic acid/CBGVA: For M31, the time course was 0.5, 1, 1.5, and 2 minutes. For M23, the time course was 5, 10, 15, and 20 minutes, and for WT NphB the time course was 8, 16, 24, and 32 minutes. The enzyme concentration for the mutants was ˜27 μM, and the concentration of WT NphB was ˜35 μM.

GC-MS characterization of isomer profile from WT NphB and M23. Samples were dissolved in 200 μL of ethyl acetate. GC-MS measurements were carried out using an Agilent Model 7693 Autosampler, 7890B Gas Chromatograph, and 7250 Q-TOF Mass Selective Detector in the Electron Ionization mode. Sample injection was carried out in split mode with inlet temperature set to 280° C. Separation was carried out on an Agilent HP5-MS column with dimensions 30 m×250 m×0.25 m. Ultra High Purity Grade He (Airgas) was used as carrier gas with the flow set to 1.1 mL/min in constant flow mode. The initial oven temperature was set to 120° C. for 1 min followed by a 20° C./min ramp to a final temperature of 300° C., which was maintained for 4 min. A 3.0 min solvent delay was used. EI energy was set to 15 eV. The MSD was set to scan the 50-500 m/z range. Data collection and analysis were performed using Mass Hunter Acquisition and Qualitative Analysis software (Agilent).

Due to the increased temperature of the GC inlet, CBGA undergoes spontaneous decarboxylation as described by Radwan et al, resulting in an M+ ion at 316 m/z. The retention time corresponding to the 316 m/z ion for the CBGA standard was 10.48 minutes.

Nonane-flow system for the extraction of CBGA from solution. A PyOx/PTA reaction was set up as detailed above. A 500 μL nonane overlay was added to the reaction in a 2 ml glass vial which was covered with 2 layers of breathable cell culture film. 2 needles were inserted into a 15 mL falcon tube at the ˜750 μL mark and the 3.5 mL mark. Luer locks to tubing connectors were connected to the needles and Viton tubing was connected to the other end of the luer lock. Needles were connected to the other end of the tubing via a luer lock connector and inserted through the mesh covering so they were only touching the nonane layer and not the reaction. 2 mL of Tris buffer [pH 8.5] was added to the 15 mL conical tube, and 6 mL of nonane was added. The nonane was pumped through the system using a peristaltic pump such that the nonane flowed from the top of the reaction through the buffered solution. The nonane pumped into the reservoir separated into the top layer of the 15 mL conical tube. The nonane from the top of the 15 mL conical tube was pumped into the top of the reaction vial. This essentially diluted the CBGA throughout the system driving the diffusion of CBGA into the nonane layer and out of the reaction.

Cloning CBDAS. A gene block of CBDAS was ordered from IDT codon optimized for Pichia pastoris. The signal sequence was removed by PCR amplifying from the 28^{th} residue of the protein sequence (NPREN . . . ) through the end of the protein, with overhangs compatible with the pPICZa vector. The PCR product was cloned into the pPICZa vector digested with EcoRI and XbaI using the Gibson cloning method. The product of the assembly reaction was transformed into BL21 Gold (DE3) cells a clone with the correct sequence isolated. The plasmid was digested with Pmei for 2 hours, and then purified using the Qiagen PCR purification protocol. The plasmid was transformed into Pichia pastoris X33 using electroporation. Immediately following electroporation, the cells were incubated in 1 mL of cold 1 M sorbitol and 1 mL of YPD media without shaking for 2 hours. The cells were plated on YPDS plates with 500 g/mL of zeocin. Colonies were screened using PCR for the presence of the CBDAS gene between the AOX1 promoter and terminator. For screening, the colonies were re-suspended in L of sterile water and 5 μL of the resuspended colony was transferred into a PCR tube with 0.2% SDS. The samples were heated for 10 minutes at 99° C., and then 1 μL was used as the template for PCR. Six colonies with positive colony PCR hits were screened for the expression of CBDAS.

CBDAS Expression Test. The six colonies were grown overnight at 30° C. to obtain a saturated culture. The overnight cultures were used to inoculate a 25 mL culture in BMGY media and grown to an OD of ˜2. The cells were harvested by centrifugation at 2,000×g for 10 minutes. The cell pellet was re-suspended in 90 mL of BMMY media, and incubated at 30° C. for 5 days. Each day, 1 mL of the culture was removed for SDS-PAGE analysis, and 500 μL of methanol was added. On day 3 the cultures were screened for CBDAS activity. The assay conditions were as follows: 100 μL of 200 mM citrate buffer, 100 μM CBGA, 5 mM MgCl₂, 5 mM KCl, 1 mM FAD, and 50 μL of the expression media in a final volume of 200 μL. The reactions were incubated overnight at room temperature and then extracted 3 times with 200 μL of ethyl acetate. The ethyl acetate extractions were pooled for each sample, and removed using a vacuum centrifuge. The samples were re-suspended in 200 μL of methanol and analyzed by HPLC. All clones produced active CBDAS.

The culture from three clones (˜300 mL total), was collected to obtain CBDAS activity. The cells were pelleted by centrifuging at ˜3,000×g for 20 minutes at 4° C. Then the supernatant was passed through a 0.22 m filter. The media was concentrated and buffer exchanged into 100 mM citrate buffer pH 5.0 using a 50,000 MWCO protein concentrator from Millipore. The total protein in the media concentrate was determined to be 0.4 mg/mL using a Bradford assay, for a total yield of ˜5 mg/L total protein.

Production of CBDVA and CBDA. To convert the precursors CBGA and CBGVA into CBDA and CBGVA respectively, a secondary reaction was set up with CBDAS synthase.

For CBGA/CBDA: A PyOx/PTA enzymatic system was set up as detailed above to produce CBGA. After 24 hours 200 μL of the nonane overlay from the CBGA reaction was transferred to a CBDAS reaction vessel. In the aqueous layer: 50 mM Hepes [pH 7.0], 5 mM MgCl₂, 5 mM KCl, 25 μM FAD, and 0.1 mg/mL CBDAS concentrate. The reaction was incubated at 30° C. with gentle shaking. Reactions were quenched at 12, 24, 48, 72, and 96 hours.

For CBGVA/CBDVA: HPLC purified CBGVA was converted to CBDVA. The final reaction volume was 200 μL, with 50 mM Hepes [pH 7.0], 5 mM MgCl₂, 5 mM KCl, 25 μM FAD, and 0.1 mg/mL (total protein) of CBDAS concentrate. A 200 μL nonane overlay was added, and the reactions were incubated at 30° C. with gentle shaking. The reactions were quenched at ˜24, 48, 72, and 96 hours.

MatB Activity Assay. A coupled enzymatic assay was used to determine the activity of malonyl-CoA synthetase (MatB) from R. palustris (see, e.g., SEQ ID NO:82-83) in the presence of OA and DA. The reaction conditions were: 2.5 mM malonate, 2 mM ATP, 1 mM CoA, 2.5 mM phosphoenolpyruvate (PEP), 1 mM NADH, 5 mM MgCl₂, 10 mM KCl, 0.35 mg/mL ADK, 0.75 μg/mL MatB, 1.6 units of PK and 2.5 units of LDH, and 50 mM Tris [pH 8.0]. Background ATPase activity was controlled for by leaving out the substrate (malonate), and either 1% ethanol, 250 μM or 5 mM OA or 5 mM DA was added to the remaining reactions. The activity of MatB was determined by monitoring decreasing absorbance at 340 nm due to NADH consumption using an M2 SpectraMax. To ensure that MatB was limiting at 5 mM OA or DA, MatB was doubled to 1.5 μg/mL. The rate of the reaction doubled suggesting that MatB was the limiting component in the system. The rate of NADH consumption at 5 mM OA and 5 mM DA was normalized to the 1% ethanol control.

Met and d3-Met Activity Assay. An enzymatic activity was conducted to compare the activity of methionine adenosyltransferase (MAT) from Thermococcus kodakarensis (tk) and Methanocaldococcusjannaschii (mj) on methionine (met), d3-met, and the production of S-adenosyl-L-methionine-d3 (SAM-d3). The reaction conditions were: 200 μl reactions containing 200 mM Tris-HCl (pH=8), 20 mM MgCl₂, 50 mM KCl, 5 mM ATP, 5 mM met or d3-met. Enzyme concentration of each MAT was 10 μM in each of four samples, for a total of eight samples. PPase (2.5 μM) was added to two samples of each MAT type. The reaction mixture was incubated for 1 h at room temperature and quenched with freshly prepared Malachite Green working solution (Sigma-Aldrich Malachite Green Phosphate Assay Kit) and measured on a plate reader at 620 nm alongside a phosphate standard according to the manufactures protocol. Enzyme activity was quantified as inorganic phosphate (Pi) released per hour.

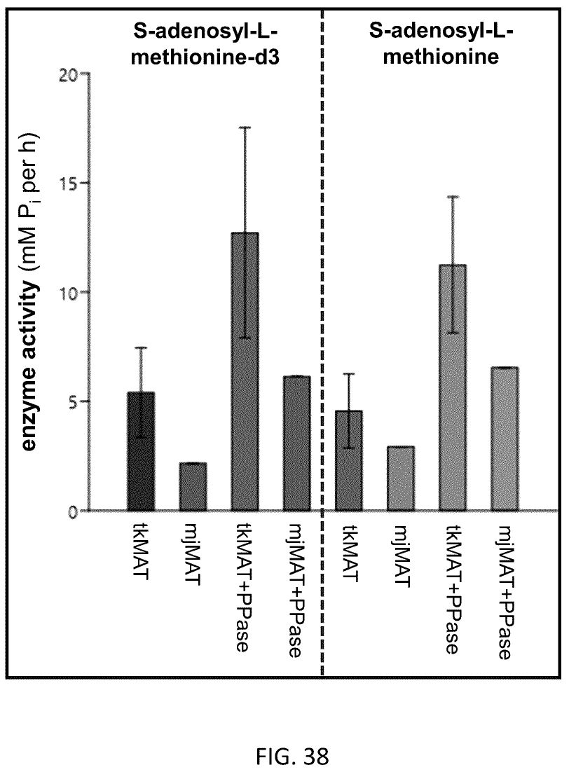

The results of this activity assay, as illustrated in FIG. 38, indicate that both methionine adenosyltransferase (MAT) from Thermococcus kodakarensis (tk) and Methanocaldococcusjannaschii (mj) are equally active on met and d3-met. As expected, adding PPase increased the amount of Pi released per hour. This overall confirms the production of SAM-d3.

AAE3 Activity Assay. A coupled enzymatic assay, similar to the one above with respect to MatB, was used to determine the activity of acyl activating enzyme 3 (AAE3) (see, e.g., SeQ ID NOs: 70-71 and homologs-SEQ ID NO:72-75) in the presence of OA and DA. The conditions were the same as the MatB assay with the following modifications: 2.5 mM hexanoate was added in lieu of malonate, and 15 μg/mL of AAE3 was added in lieu of MatB. To ensure that AAE3 was limiting, AAE3 was doubled in the presence of 5 mM OA or DA. The rate of the reaction doubled indicating AAE3 is limiting.

ADK Activity Assay. A coupled enzymatic assay was used to determine the activity of adenylate kinase (ADK) (see, e.g., SEQ ID NO: 81) in the presence of OA and DA. The conditions were similar to the MatB assay, with the following modifications: 2 mM AMP was added in lieu of malonate, CoA was not added, and 0.001 mg/mL of ADK was added. To ensure that ADK was the limiting reagent at 5 mM OA and DA, the amount of ADK was doubled. The 2-fold increase in rate suggested that ADK was the limiting factor.

CPK Activity Assay. A coupled enzymatic assay was used to determine the activity of creatine kinase (CPK) in the presence of OA or DA. The reaction conditions were: 5 mM Creatine Phosphate, 2 mM ADP, 5 mM glucose, 2 mM NADP⁺, 5 mM MgCl₂, 5 mM KCl, 0.3 mg/mL Zwf, 0.1 mg/mL Sc Hex, and 0.08 units CPK. The positive control reaction contained 1% ethanol, and either 5 mM of OA or DA was added to the remaining reactions. The absorbance of NADPH at 340 nm was monitored. To ensure that CPK was limiting was doubled at 5 mM OA and 5 mM DA. The resulting rate doubled, which indicates CPK is limiting even at high OA and DA.

OLS Activity Assay. Olivetol synthase (OLS) (see, e.g., SEQ ID NO:76-77) was assayed by setting up the following conditions: 200 μM malonyl CoA, 100 μM hexanoyl-CoA, 0.65 mg/mL OAS, in either 50 mM citrate buffer pH 5.5 or 50 mM Tris buffer pH 8.0. The reactions were initiated by the addition of OAS, and then they were quenched at 30 minutes by adding 150 μL of methanol to the 50 μL reaction. The samples were centrifuged at ˜16,000×g for 2 minutes to pellet the proteins. The supernatant was analyzed using the HPLC.

For the inhibition experiments the conditions were altered to: 1 mM malonyl-CoA, 400 μM hexanoyl-CoA in 50 mM citrate buffer, pH 5.5 in a final volume of 200 μL. Either 1% ethanol, 250 μM OA or 1 mM DA was added to the reaction, and then the reactions were initiated by adding 0.65 mg/mL OLS. 50 μL aliquots were quenched at 2, 4, 6, and 8 minutes in 150 μL of methanol. The reactions were vortexed briefly and centrifuged at 16,000×g for 2 minutes to pellet the proteins. The supernatant was analyzed by HPLC. The raw peak areas of HTAL, PDAL, and olivetol were summed and plotted against time to determine the rate. The rate of the OA supplemented reaction and the DA supplemented reaction were normalized to the ethanol control.

OLS/OAC Activity Assay. To produce OA, the same OLS conditions specified above were used, but olivetolic acid cyclase (OAC) (see, e.g., SEQ ID NO:78-79) was added to the reaction at 0.6 mg/mL. The reactions were quenched and analyzed in the same manner as the OLS assay. Acetyl-phosphate and BSA were added to the assays individually 5 mM-40 mM AcP and 10-30 mg/mL BSA final concentration.

Full pathway set up. The enzymes used in this study and the final concentration (mg/mL) are found in Table 6 for the MatB path and Table 7 for the MdcA path. For the MatB path, the cofactors were added at the following concentrations: 150 mM glucose, 1 mM fructose bisphosphate, 2 mM ATP, 0.25 mM NAD+, 3 mM NADP+, 2 mM CoA, 0.25 mM 2,3-bisphosphoglycerate, 6 mM MgCl₂, 10 mM KCl, 0.5 mM thiamine pyrophosphate, 50 mM phosphate pH 8.0, 5 mM hexanoate, 15 mM malonate, 5 mM creatine phosphate, and 50 mM Tris, pH 8.0. The reactions were initiated by the addition of the enzymes listed in Table 6. The reaction was incubated overnight at room temperature, and the reaction was quenched and extracted 3 times with 200 μL of ethyl acetate. The ethyl acetate was removed using a vacuum centrifuge. The sample was dissolved in 200 μL of methanol and analyzed using HPLC.

TABLE 6

| | |
|---|---|
| Enzyme | mg/mL |

Glycolysis

| Sc Hex | 0.02 | |
|---|---|---|
| Gs PfkA | 0.32 | |
| Sa Fba | 0.18 | |
| Gs TpiA | 0.04 | |
| Gs Pgi | 0.17 | |
| Ec GapA | 0.05 | |
| Gs GapM6 | 0.18 | |
| Gs Pgk | 0.03 | |
| Ec dPgm | 0.38 | |
| Ec Eno | 0.08 | |
| Ec PykF | 0.56 | |
| Av PyOx | 1 | unit |
| Gs PTA | 0.06 | |
| LI NoxE | 0.25 | |
| Ca Catalase | 125 | units |

Mevalonate

| | |
|---|---|
| Re PhaA | 0.12 |
| Ef HMGS-A110G | 0.22 |
| Ef HMGR | 0.58 |
| Mm MVK | 0.16 |
| Spne PMVK | 0.23 |
| Spne MDC | 0.22 |
| Ec IDI | 0.23 |
| Gs FPPS S82F | 0.04 |
| SCL190 NphB | 0.45 |
| Gs PPase | 0.16 |

Olivetolate

| Rp MatB | 0.03 | |
|---|---|---|
| Cs AAE3 | 0.18 | |
| Cs OLS | 0.25 | |
| Cs OAC | 0.87 | |
| Gt ADK | 0.07 | |
| Creatine Kinase | 2 | units |

TABLE 7

| | |
|---|---|
| Enzyme | mg/mL |

Glycolysis

| Sc Hex | 0.02 | |
|---|---|---|
| Gs PfkA | 0.32 | |
| Sa Fba | 0.18 | |
| Gs TpiA | 0.04 | |
| Gs Pgi | 0.17 | |
| Ec GapDH | 0.05 | |
| Gs GapM6 | 0.18 | |
| Gs Pgk | 0.03 | |
| Ttg dPgm | 0.09 | |
| Ec EnoNH | 0.08 | |
| Gs PykA | 0.13 | |
| Av PyOx | 1 | unit |
| Gs PTA | 0.06 | |
| LI NoxE | 0.25 | |
| Ca Catalase | 125 | units |

Mevalonate

| | |
|---|---|
| Re PhaA | 0.12 |
| Ef HMGS A110G | 0.43 |
| Ef HMGR | 0.58 |
| Mm MVK | 0.16 |
| Spne PMVK | 0.23 |
| Spne MDC | 0.19 |
| Ec IDI-CH | 0.23 |
| Gs FPPS S82F | 0.04 |
| SCL190 NphB 31 | 0.68 |
| Gs Ppase | 0.16 |

Olivetolate

| Gs MdcA | 0.18 | |
|---|---|---|
| Cs AAE3 | 0.12 | |
| Cs OAS | 0.60 | |
| Cs OAC | 0.87 | |
| Gt ADK | 0.07 | |
| Creatine Kinase | 2 | units |

The pathway of both the MatB and MdcA pathway are provided in FIG. 6A-B.

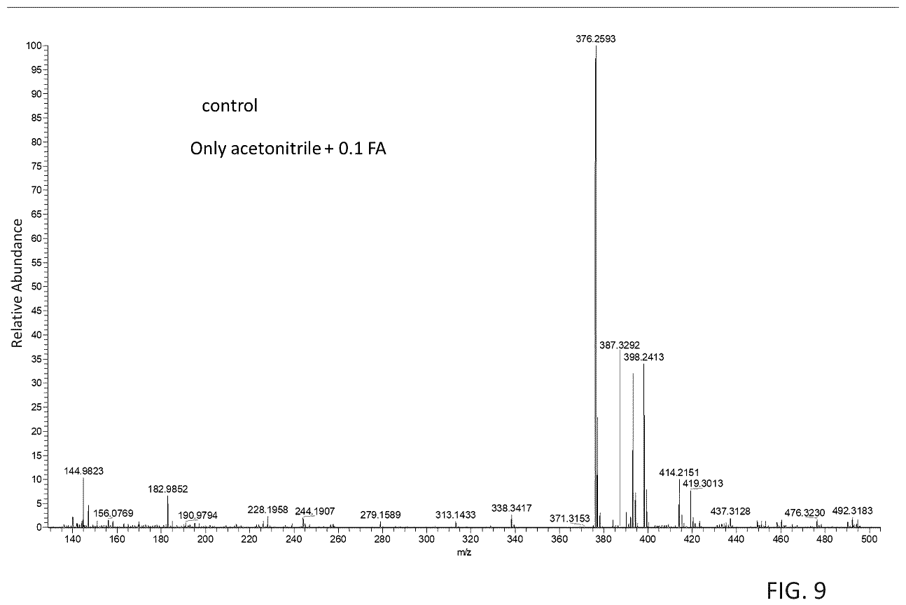

FIG. 9 illustrates Fourier transform mass spectrometry with probe electrospray ionization (FTMS-pESI) spectral data for acetonitrile with 0.1% formic acid as a control.

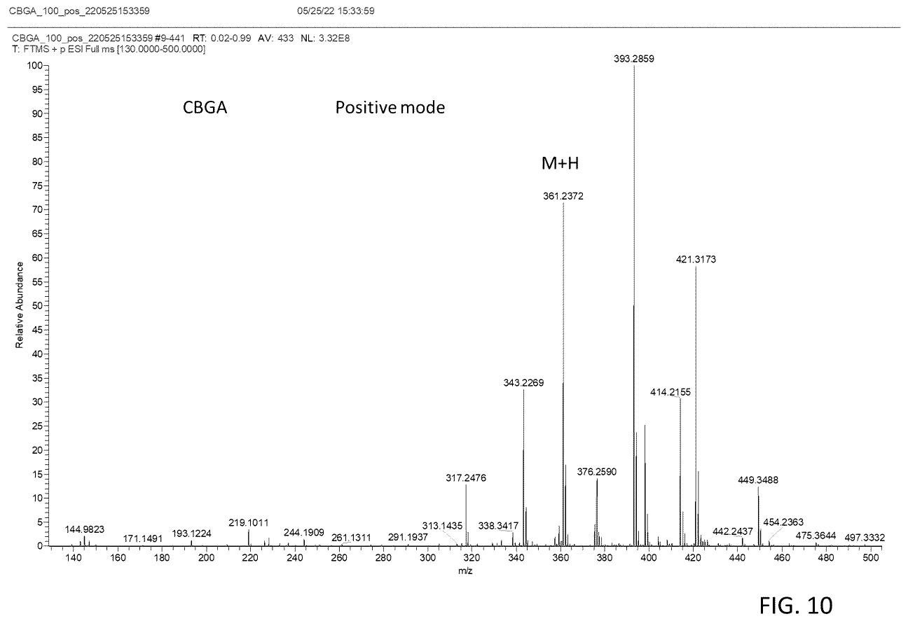

FIG. 10 illustrates FTMS-pESI spectral data for CBGA in positive mode.

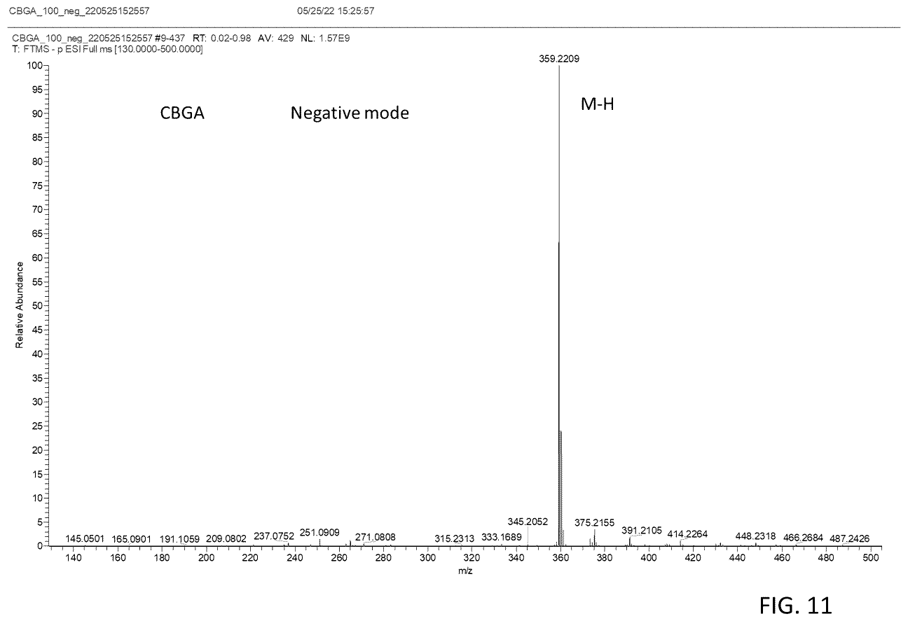

FIG. 11 illustrates FTMS-pESI spectral data for CBGA in negative mode.

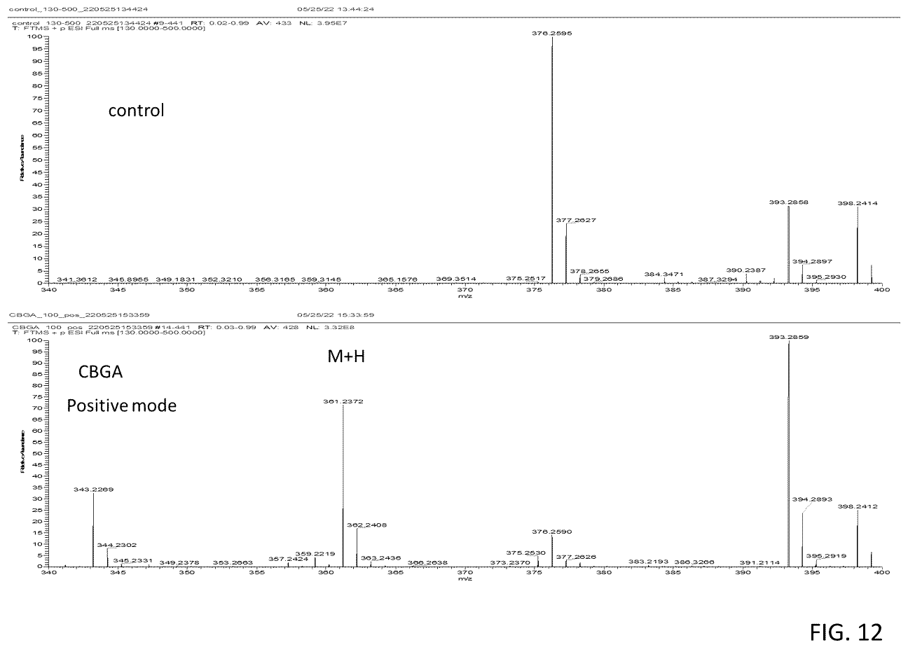

FIG. 12 illustrates FTMS-pESI spectral data for the control and CBGA in positive mode.

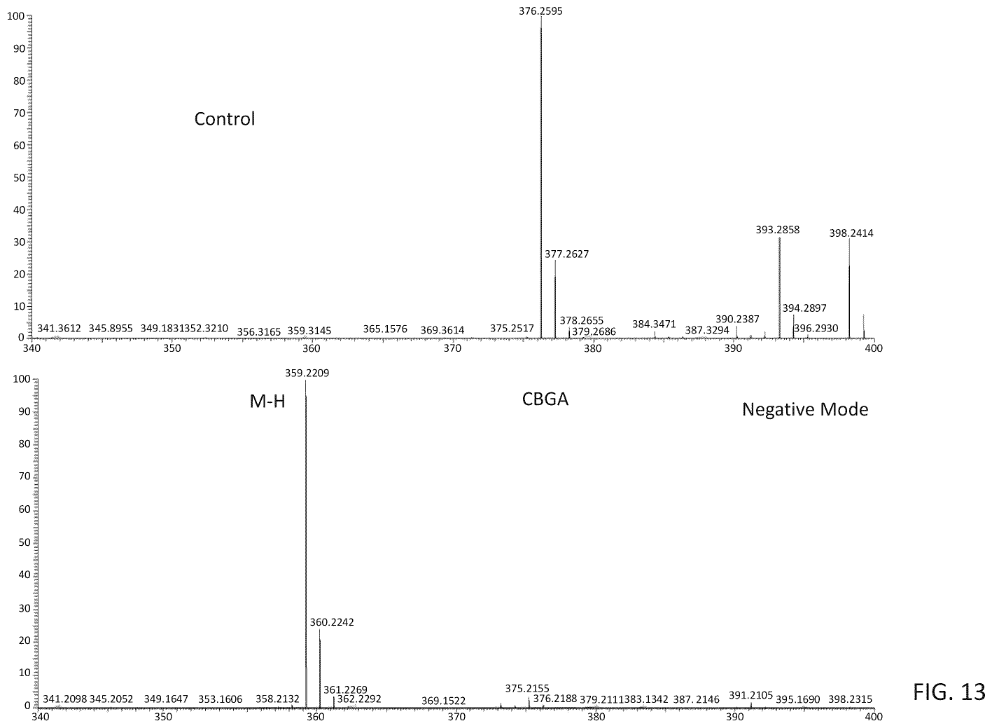

FIG. 13 illustrates FTMS-pESI spectral data for the control and CBGA in positive mode.

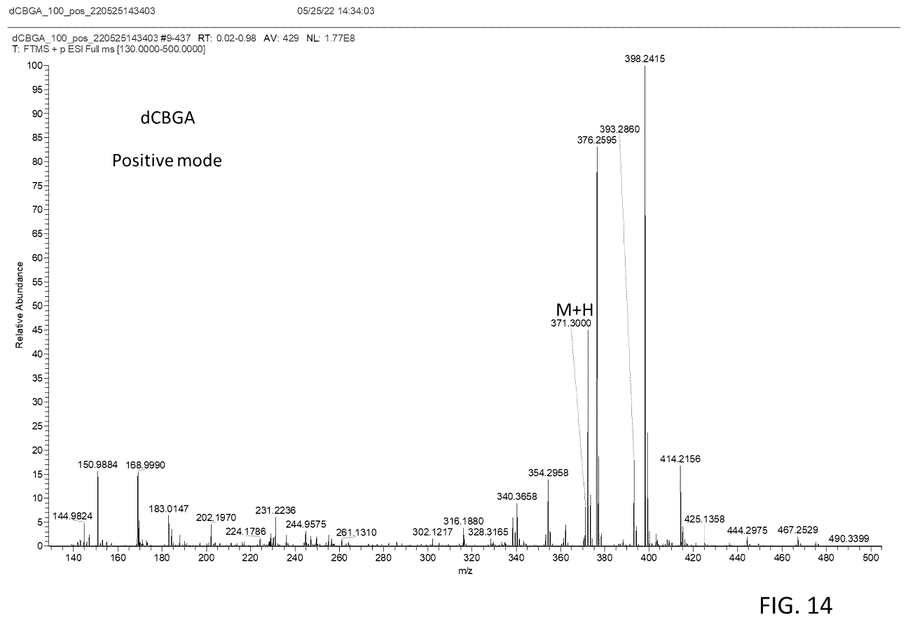

FIG. 14 illustrates FTMS-pESI spectral data for deuterated CBGA in positive mode.

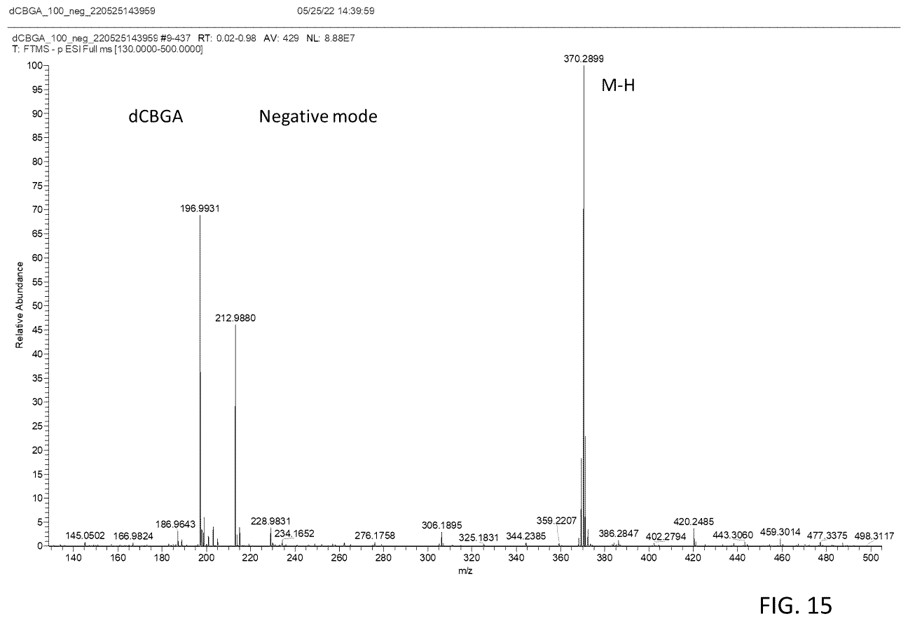

FIG. 15 illustrates FTMS-pESI spectral data for deuterated CBGA in negative mode.

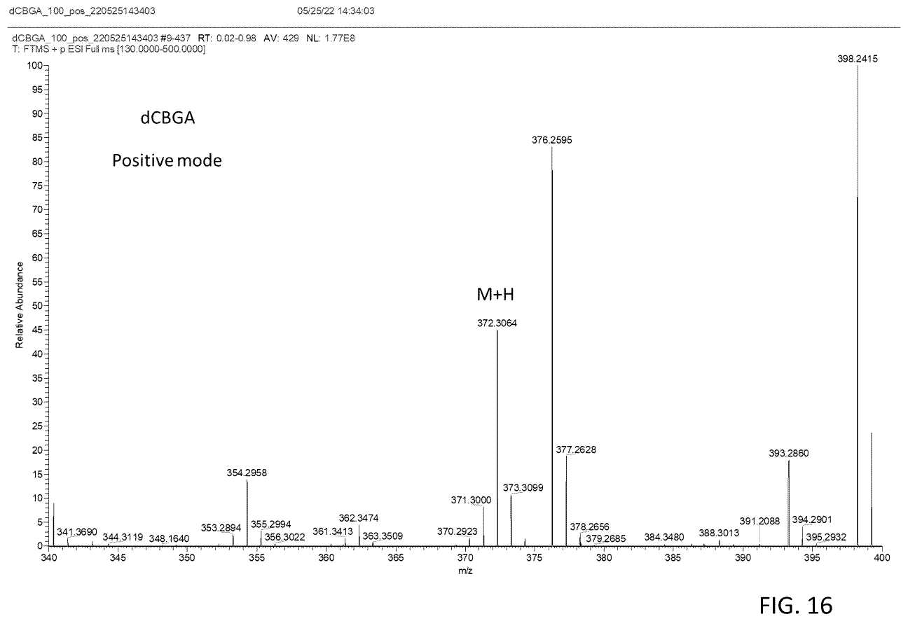

FIG. 16 illustrates additional FTMS-pESI spectral data for deuterated CBGA in positive mode.

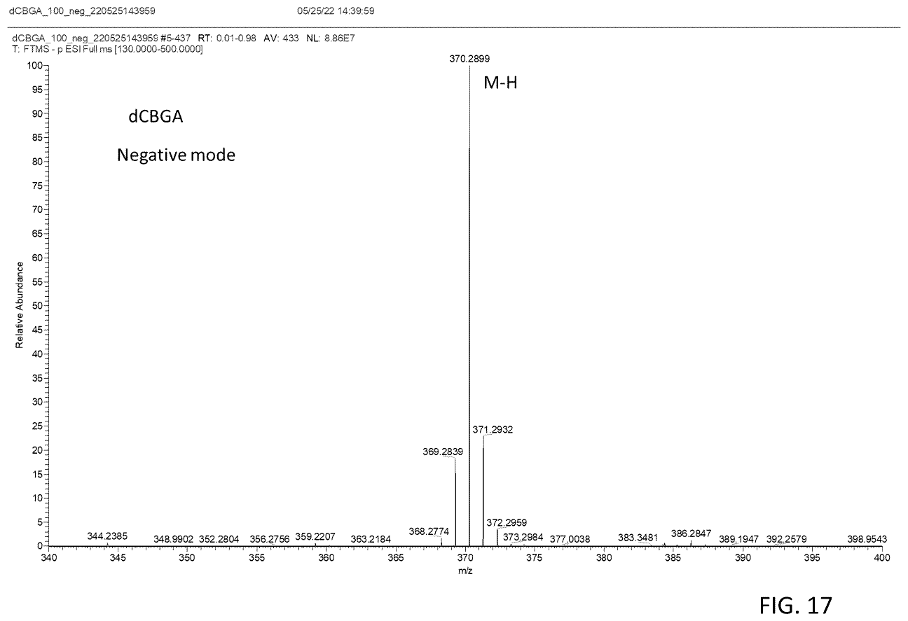

FIG. 17 illustrates additional FTMS-pESI spectral data for deuterated CBGA in negative mode.

Table 8 shows the results of the FTMS-pESI spectral analysis, which indicate that compounds are operable to be identified in positive and negative modes.

TABLE 8

| | Positive | Negative |
|---|---|---|
| CBGA | 361.2372 | 359.2209 |
| dCBGA | 372.3064 | 370.2899 |

Additional samples were subjected to nano-scale liquid chromatographic tandem mass spectrometry (nLC-MS/MS). Samples were prepared using 2 μL of standard and 198 μL of 50:50 water:acetonitrile in 0.1% formic acid. A 1 μL sample was injected.

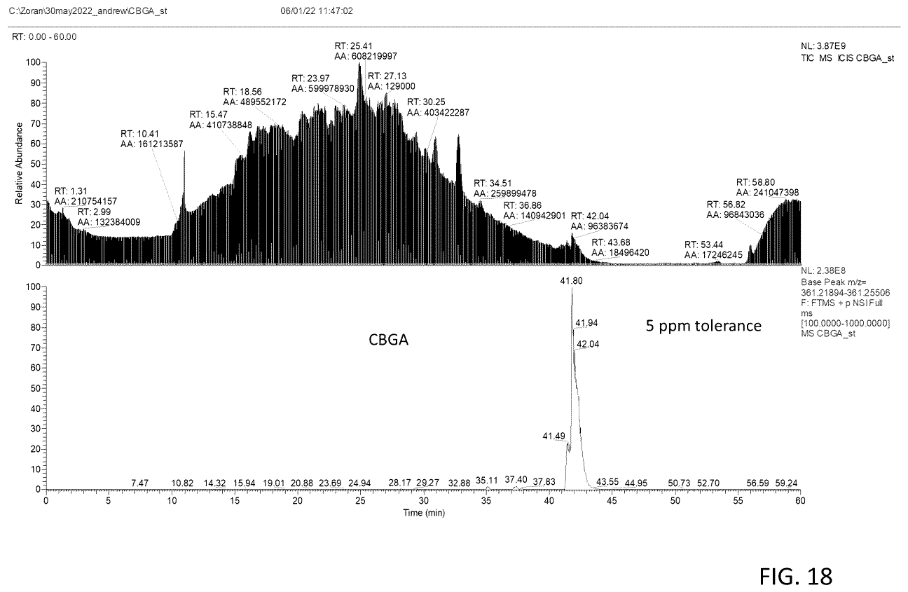

FIG. 18 illustrates nLC-MS/MS data for CBGA.

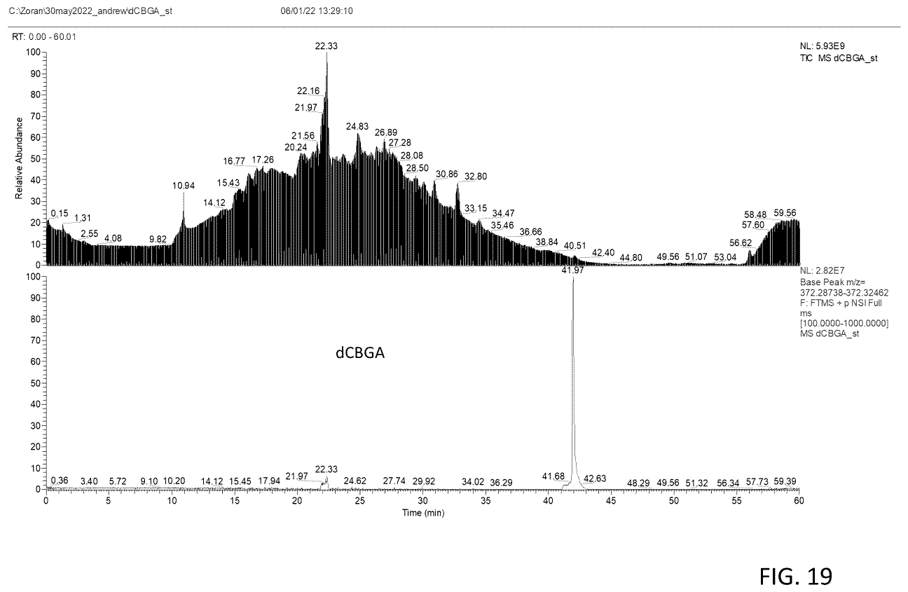

FIG. 19 illustrates nLC-MS/MS data for dCBGA.

Table 9 compares nLC-MS/MS data for CBGA and dCBGA.

TABLE 9

| | Positive | Negative | Retention time (min) |
|---|---|---|---|
| CBGA | 361.2372 | 359.2209 | 41.5-43.0 |
| dCBGA | 372.3064 | 370.2899 | 41.97 ± 0.5 |

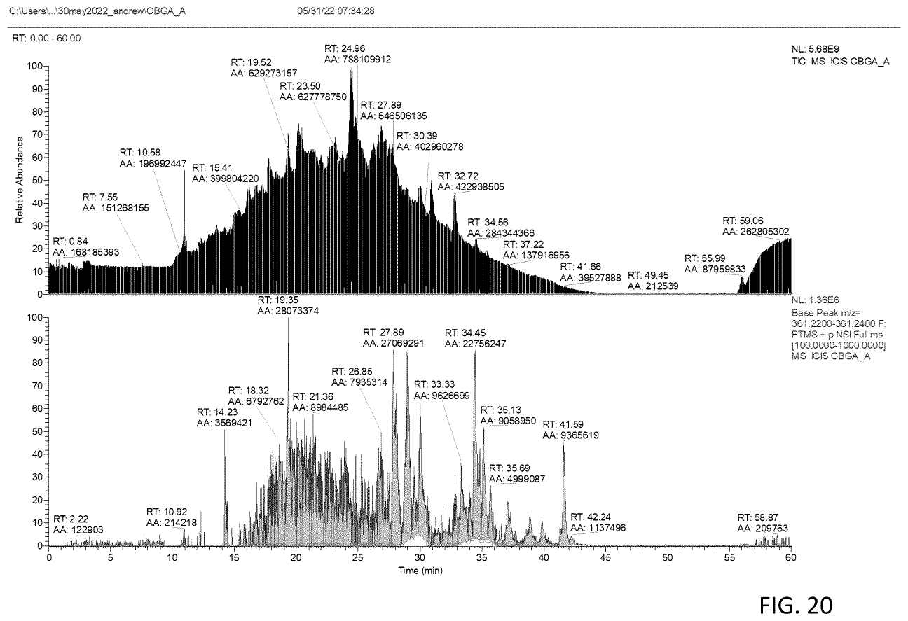

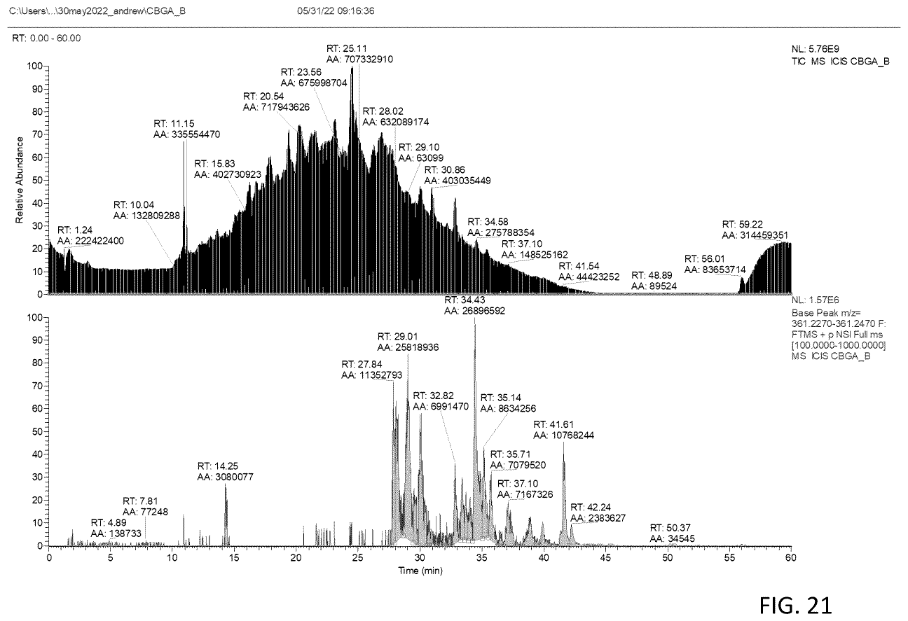

FIG. 20 illustrates nLC-MS/MS data for a first sample of CBGA after one hour.

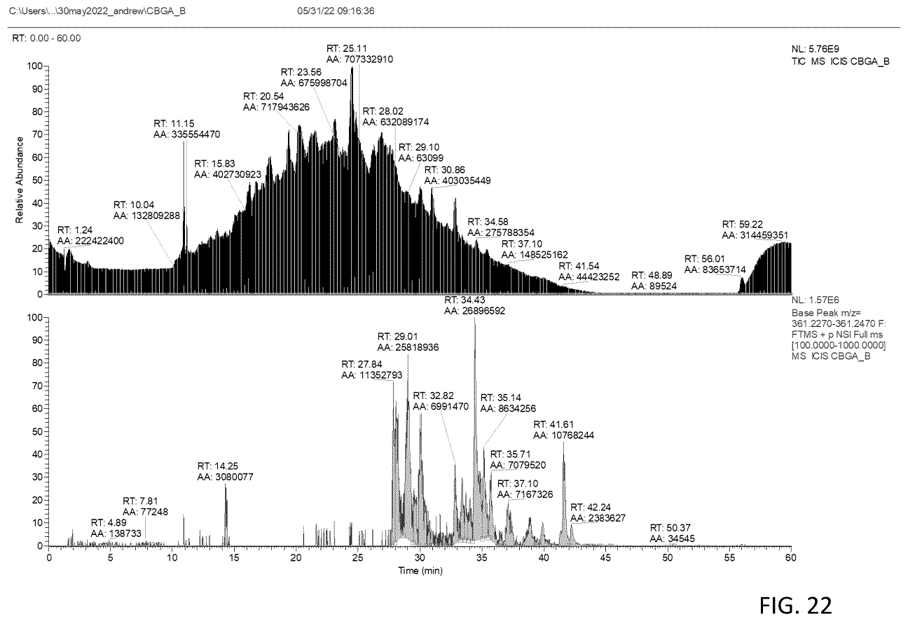

FIG. 21 illustrates nLC-MS/MS data for a second sample of CBGA after one hour.

FIG. 22 illustrates nLC-MS/MS data for a third sample of CBGA after one hour.

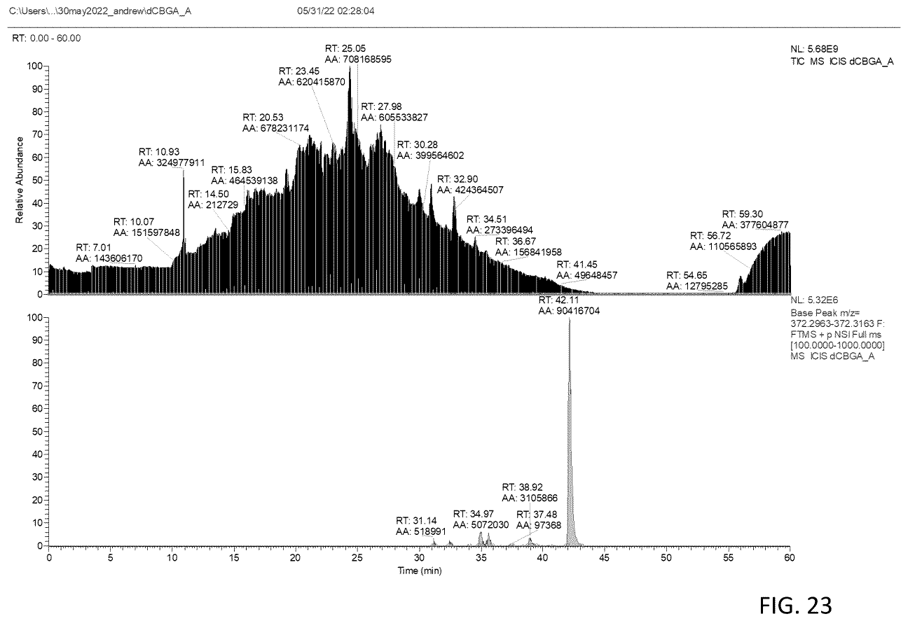

FIG. 23 illustrates nLC-MS/MS data for a first sample of dCBGA after one hour.

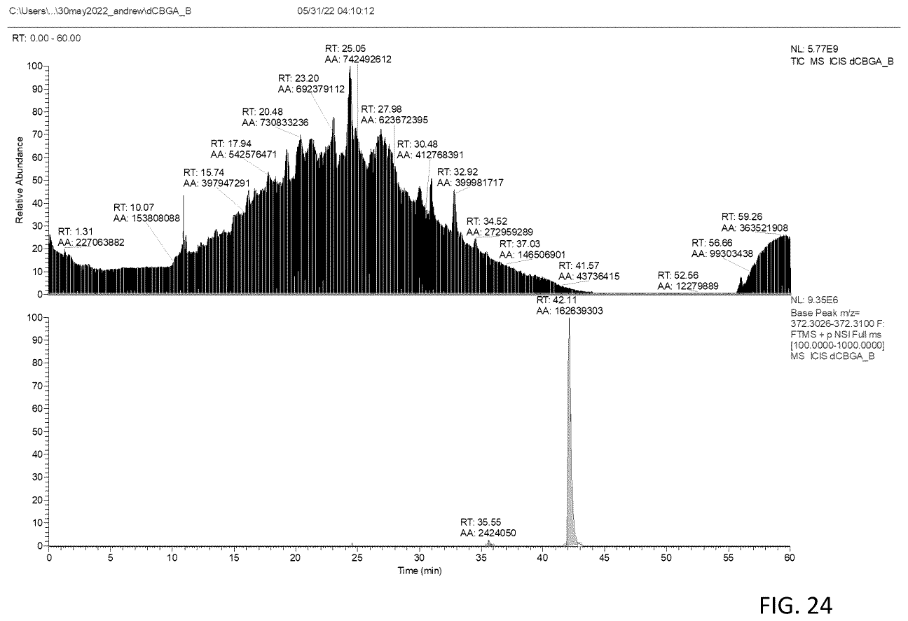

FIG. 24 illustrates nLC-MS/MS data for a second sample of dCBGA after one hour.

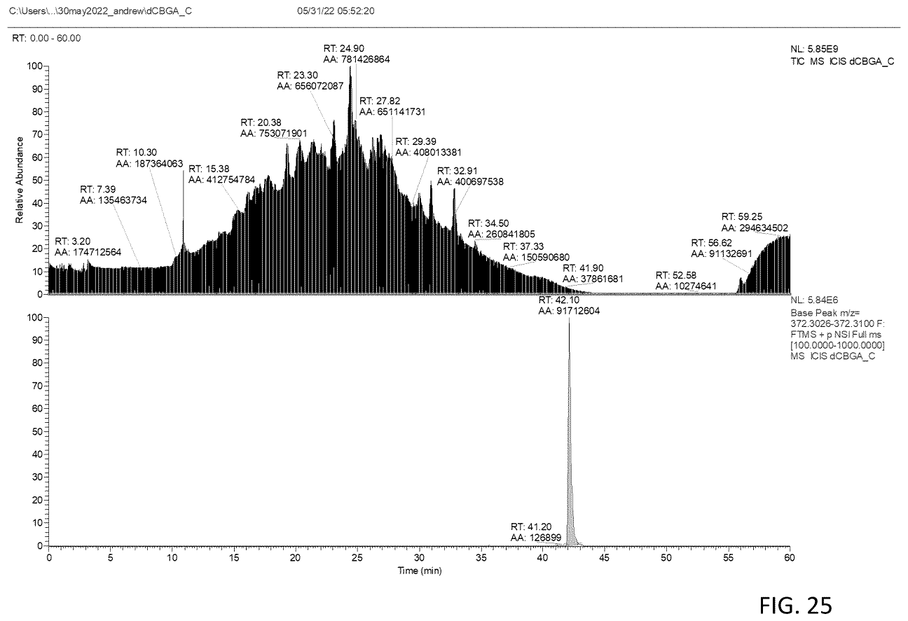

FIG. 25 illustrates nLC-MS/MS data for a third sample of dCBGA after one hour.

Table 10 compares nLC-MS/MS data for the CBGA samples and dCBGA samples after one hour.

TABLE 10

| CBGA | dCBGA |
|---|---|
| 9365619 | 90416704 |
| 10768244 | 162639303 |
| 8328277 | 91712604 |

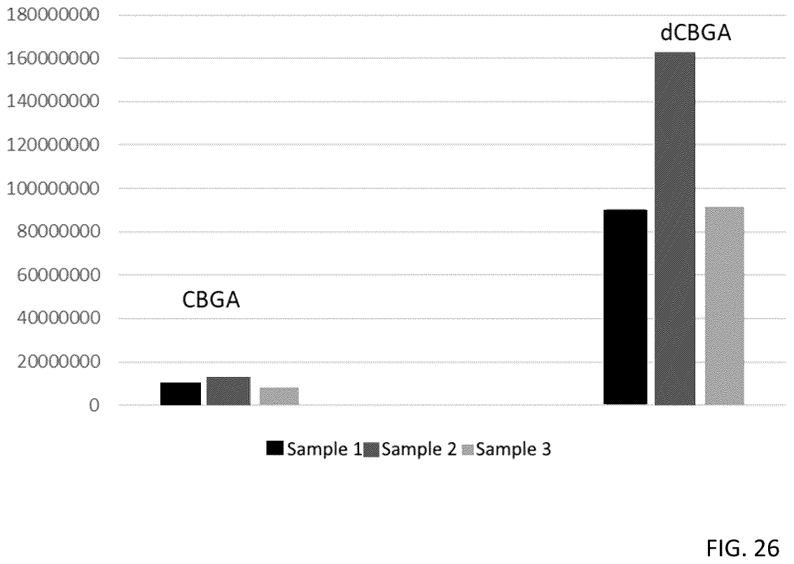

FIG. 26 illustrates a graph comparing CBGA and dCBGA. Sample 1, Sample 2, and Sample 3 are shown left to right for both CBGA and dCBGA. The results indicate that CBGA is significantly more degraded after one hour than dCBGA.

In one embodiment, upon formation of the THCA compound of the present invention, the compound is purified to isolate the produced THCA. The extraction of CBGA and THCA into ethylacetate formed an emulsion which could only be partially broken with the addition of hexane. Bovine serum albumin (BSA) binds both CBGA and THCA tightly and thus not all of the product is recoverable in the organic phase of the purification process. However, acid precipitation of the BSA with the addition of 1% HCl allows for the denaturation of BSA and subsequent unbinding of the CBGA and THCA to allow extraction and purification. In one embodiment, the HCl is added directly to the reaction mixture, which is then incubated for about 10 minutes to assure full denaturation of BSA. The mixture is then centrifuged for about 30 minutes at 4000 g. After centrifuging the mixture, the CBGA and THCA coprecipitate into pelleted form. The pellet is then washed with water until the water reaches a neutral pH. The pellet is then resuspended in methanol using vigorous vortexing. The methanol mixture was rotavapped over a water bath at 40° C., and the resulting oil was resuspended in ethanol. The THCA then precipitates as a brown oil. The reaction mixture is then filtered and washed with water and then resuspended in hexane. The hexane was then rotavapped to remove mass added to the product by excess methane. The resulting oil was resuspended in methanol for storage. Table 11 below shows the summary of recovered product during the full step starting with 67.5 mg of CBGA reaction mix. In the experiment detailed in Table 11, 41 mg of purified product was obtained using this purification process, which is 60% of the theoretical yield. In one embodiment, the implementation of a crystallization step avoids the ethanol precipitation, thereby improving the purity and recovery of THCA.

TABLE 11

| | Product Amount [mg] | % Recovered |
|---|---|---|
| CBGA reaction mix | 67.5 | |
| Conversion to THCA | 58.5 | 86.7 |
| Methanol extraction | 47.6 | 70.5 |
| Final product | 41 | 60.7 |

Upon purification of the THCA, the purified THCA is further operable to be decarboxylated to obtain THC. In the continuation of the experiment described above with respect to Table 11, 23 mg of the purified THCA were used for decarboxylation. The methanol was rotavapped off the product and the product was subsequently resuspended in 2 mL of toluene. Next, 100 uL of saturated bicarbonate was added to the mixture and the mixture was heated to 95° C. on the rotavap to allow for refluxing and condensation. Samples were taken after 10 minutes and 35 minutes at which point the reaction was stopped. The reaction mixture was washed with water to remove excess salts. Ethanol was then added to the toluene to further assist in the evaporation of excess reactants and rotavapped dry three times to remove toluene. This process yielded 13.4 mg of THC at 90% purity.

In one embodiment, impurities in the compound resulting in increased retention time are operable to be removed prior to decarboxylation through THCA crystallization in an ethanol/hexane mixture.

One Pot Synthesis

In one embodiment, the synthesis is a one-pot synthesis. In a one-pot synthesis, the synthesis reactions are carried out by combining all reagents, catalysts, enzymes, buffers, and other necessary chemical specials in a single reaction vessel. In one embodiment, the one-pot synthesis includes at least one deuterated compound (e.g., a deuterated fatty acid). Advantageously, a one-pot synthesis improves the efficiency of a chemical reaction, thereby increasing chemical yield.

Formulations

In one embodiment, the cannabinoid, the cannabinoid precursor, and/or the other prenylated chemical substituted with at least one deuterium, at least one tritium, at least one halogen, at least one hydroxyl group, and/or at least one additional isotope are incorporated into a pharmaceutical composition. In one embodiment, the pharmaceutical composition includes about 1% (w/w) or more of the cannabinoid, the cannabinoid precursor, and/or the other prenylated chemical substituted with the at least one deuterium, the at least one tritium, the at least one halogen, the at least one hydroxyl group, and/or the at least one additional isotope (e.g., about 2% (w/w) or more, about 3% (w/w) or more, about 4% (w/w) or more, about 5% (w/w) or more, about 6% (w/w) or more, about 7% (w/w) or more, about 8% (w/w) or more, about 9% (w/w) or more, about 10% (w/w), about 15% (w/w), about 20% (w/w) or more, or about 25% (w/w) or more).

In one embodiment, the pharmaceutical composition further includes an unsubstituted cannabinoid, an unsubstituted cannabinoid precursor, and/or an unsubstituted other prenylated chemical. In one embodiment, the pharmaceutical composition includes about 1% (w/w) or more of the unsubstituted cannabinoid, the unsubstituted cannabinoid precursor, and/or the unsubstituted other prenylated chemical e (e.g., about 2% (w/w) or more, about 3% (w/w) or more, about 4% (w/w) or more, about 5% (w/w) or more, about 6% (w/w) or more, about 7% (w/w) or more, about 8% (w/w) or more, about 9% (w/w) or more, about 10% (w/w), about 15% (w/w), about 20% (w/w) or more, or about 25% (w/w) or more).

In one embodiment, the pharmaceutical composition further includes a lipid. The lipid includes, but is not limited to, a phospholipid (e.g., soy lecithin, egg lecithin, phosphocholines, phosphoglycerols), fat, oil (e.g., olive oil, vegetable oil), and/or a fatty acid. In one embodiment, the lipid is operable to form micelles, emulsions, or liposomes.

In one embodiment, the cannabinoid, the cannabinoid precursor, and/or the other prenylated chemical substituted with at least one deuterium, at least one tritium, at least one halogen, at least one hydroxyl group, and/or at least one additional isotope are microencapsulated or nanoencapsulated.

In one embodiment, the pharmaceutical composition further includes at least one masking agent (e.g., taste masking agent, smell masking agent). In a preferred embodiment, the at least one masking agent includes, but is not limited to, at least one sweetener and/or at least one flavoring agent. The at least one sweetener includes, but is not limited to, saccharin (e.g., sodium salt, calcium salt), fructose, dextrose, aspartame, acesulfame potassium, glycerin, sucralose, maltodextrin, sucrose, glucose, maltose, xylitol, sorbitol, erythritol, and/or mannitol. In one embodiment, the at least one masking agent includes phenethyl alcohol, vanilla, cherry, cinnamon, lavender, lemon, menthol, orange, peppermint, spearmint, raspberry, strawberry, grape, ethyl vanillin, coriander, ginger, nutmeg, cardamom, butterscotch, cocoa, acacia syrup, anethole, anise oil, benzaldehyde, ethyl acetate, methyl salicylate, and/or tolu. In one embodiment, the at least one masking agent is about 0.001% to about 1% w/w of the weight of the pharmaceutical composition, for example about: 0.001%, 0.005%, 0.01%, 0.02%, 0.05%, 0.1%, 0.2%, 0.3%, 0.4%, 0.5%, 0.6%, 0.7%, 0.8%, 0.9%, or 1% w/w based on the weight of the pharmaceutical composition. In one embodiment, the at least one masking agent is about 0.01% to 0.5%, 0.02% to 0.2%, or 0.015% to 0.15% w/w based on the weight of the pharmaceutical composition.

In one embodiment, the pharmaceutical composition further includes a vitamin or a mineral. The vitamin includes, but is not limited to, vitamin A, vitamin C, vitamin D (e.g., vitamin D1, D2, D3, D4, D5, D6, and/or D7), vitamin E, vitamin B (e.g., B1, B2, B3, B5, B9, and/or B12), vitamin K (e.g., K1, K2, K3, K4, and/or K5). The mineral includes, but is not limited to, magnesium, calcium, iron, zinc, chrome, selenium, and/or potassium.

In one embodiment, the pharmaceutical composition further includes at least one anticaking agent. The at least one anticaking agent includes, but is not limited to, tribasic calcium phosphate, cellulose, microcrystalline cellulose, silicon dioxide, sodium chloride, magnesium stearate, magnesium carbonate, and/or sodium bicarbonate. In one embodiment, the at least one anticaking agent is about 0.5% to about 5% w/w of the weight of the pharmaceutical composition.

In one embodiment, the pharmaceutical composition includes at least one preservative. The at least one preservative includes, but is not limited to a paraben, benzalkonium chloride, phenyl ethyl alcohol, ethylenediaminetetraacetic acid (EDTA), benzoyl alcohol, sulfur dioxide, a sulfite, a thiol, propionic acid, benzoic acid, sorbic acid, sodium sorbate, calcium sorbate, potassium sorbate, sodium benzoate, potassium benzoate, lactic acid, and/or sodium propionate. In one embodiment, the at least one preservative is about 0.01% to about 5% w/w of the weight of the composition, for example about: 0.0%, 0.05%, 0.1%, 0.2%, 0.5%, 1%, 2%, 3%, 4%, or 5% w/w based on the weight of the pharmaceutical composition. In one embodiment, the at least one preservative is about 0.01% to 5%, 0.02% to 4%, or 0.05% to 2.5% w/w based on the weight of the pharmaceutical composition. In a preferred embodiment, the at least one preservative is sulfite-free.

In one embodiment, the pharmaceutical composition is in a crystalline, powder, granular, capsule, tablet, syrup, solution, emulsion, topical, wafer, aerosol, oil, patch (e.g., transdermal patch), parenteral, suppository, intravenous, or suspension form. In one embodiment, the pharmaceutical composition is delivered via oral administration, sublingual route, buccal route, rectal route, intravenous injection, intramuscular administration, subcutaneous injection, intranasal route, inhaled route, ocular route, or vaginal route. In one embodiment, the pharmaceutical composition is provided in an edible form. The edible form includes, but is not limited to, candy, pastry (e.g., brownie, cookie, muffin), beverage, bread, cereal, or pasta. The topical form includes, but is not limited to, a cream, an ointment, a lotion, a paste, or a gel.

Treatment

In one embodiment, the pharmaceutical composition is used to treat cancer (e.g., stomach cancer, colon cancer, pancreatic ductal adenocarcinoma), glaucoma, fibromyalgia, peripheral neuropathy, nausea, diabetes, obesity, a liver disease, a neurological disorder (e.g., seizure, epilepsy, multiple sclerosis, stroke, Parkinson's Disease, vascular dementia, senile dementia, Alzheimer's disease, mild cognitive impairment, Huntington's Disease, Amyotrophic Lateral Sclerosis (ALS), migraine), an autoimmune disorder (e.g., type 1 diabetes, Crohn's disease, Celiac disease, ulcerative colitis, inflammatory bowel disease (IBD), lupus, rheumatoid arthritis, psoriatic arthritis, Addison's disease, Graves' disease, vasculitis, pernicious anemia), a skin disorder (e.g., psoriasis, atopic dermatitis (AD), eczema, acne, contact dermatitis, herpes simplex, shingles, actinic keratosis, ichthyosis, Bowen's disease, keratoacanthoma, lichen sclerosus, hidradenitis suppurativa, seborrheic keratosis, rosacea, Pityriasis lichenoid, seborrhea), a joint disorder (e.g., osteoarthritis), a reproductive disorder (e.g., endometriosis, dysmenorrhea, irregular menstrual bleeding, dyspareunia), a bacterial infection, and/or a psychiatric disorder (e.g., anxiety, depression, stress, panic attacks, attention-deficit/hyperactivity disorder (ADHD, post-traumatic stress disorder (PTSD)), bipolar disorder, obsessive compulsive disorder, schizophrenia, personality disorders). See, e.g., U.S. Patent Publication No. 20210315837, which is incorporated herein by reference in its entirety.

In one embodiment, the pharmaceutical composition is administered once a day, twice a day, three times a day, or four times day. In another embodiment, the pharmaceutical composition is administered once every two days, once every three days, once every four days, once every five days, once every six days, or once every seven days. In yet another embodiment, the pharmaceutical composition is administered once every two weeks, once every three weeks, once every four weeks, once every six weeks, once every two months, once every three months, or once every four months.

Certain modifications and improvements will occur to those skilled in the art upon a reading of the foregoing description. The above-mentioned examples are provided to serve the purpose of clarifying the aspects of the invention and it will be apparent to one skilled in the art that they do not serve to limit the scope of the invention. All modifications and improvements have been deleted herein for the sake of conciseness and readability but are properly within the scope of the present invention.

The invention claimed is:

1. A cannabinoid composition comprising:
a cannabinoid derived from a deuterated fatty acid, wherein the cannabinoid is a compound of Formula I: 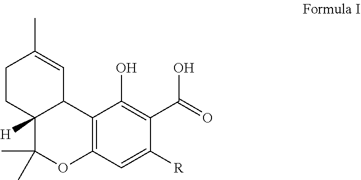
wherein R is a deuterated carbon chain; and
wherein the deuterated carbon chain includes at least one deuterated carbon.

2. The composition of claim 1, wherein the deuterated carbon chain is C5D₁₁.

3. The composition of claim 1, wherein the composition further includes an unsubstituted cannabinoid and/or an unsubstituted cannabinoid precursor.

4. The composition of claim 1, wherein the composition includes a prodrug.

5. The composition of claim 1, wherein the composition further includes a phospholipid, fat, oil, and/or a fatty acid.

6. The composition of claim 1, wherein the composition further includes at least one sweetener and/or at least one flavoring agent.

7. The composition of claim 1, wherein the composition further includes a vitamin or a mineral.

8. The composition of claim 1, wherein the composition further includes at least one anticaking agent.

9. The composition of claim 1, wherein the composition further includes at least one preservative.

10. The composition of claim 1, wherein the composition is in a crystalline, powder, granular, capsule, tablet, syrup, solution, emulsion, topical, wafer, aerosol, oil, patch, parenteral, suppository, intravenous, or suspension form.

11. A method for synthesizing a cannabinoid compound or derivative thereof, comprising:
phosphorylating prenol and/or isoprenol using hydroxyethylthiazole kinase (ThiM) to produce isopentenyl phosphate;
isomerizing isopentenyl phosphate to produce isopentenyl diphosphate, wherein isopentenyl diphosphate is operable to be phosphorylated in the presence of inositol polyphosphate kinase to produce dimethylallyl diphosphate (DMAPP);
synthesizing geranyl pyrophosphate from isopentenyl diphosphate and/or DMAPP in the presence of farnesyl pyrophosphate synthase;
activating a deuterated fatty acid to produce a deuterated CoA thioester;
activating an acid to produce a second CoA thioester;
synthesizing the deuterated CoA thioester and the second CoA thioester;
cyclizing the synthesized product to produce a deuterated olivetolic acid;
prenylating the deuterated olivetolic acid in the presence of geranyl pyrophosphate to produce a deuterated cannabigerolic acid;
cyclizing the deuterated cannabigerolic acid to produce a deuterated cannabinoid.

12. The method of claim 11, further comprising recovering the deuterated cannabinoid from a reaction mixture, wherein acid precipitation, centrifugation, washing, rotovapping, and precipitation are used to isolate the cannabinoid from the reaction mixture.

13. The method of claim 11, wherein the method for synthesizing a cannabinoid compound or derivative thereof is a one-pot synthesis.

14. The method of claim 11, wherein the deuterated fatty acid is a fully deuterated acid or a partially deuterated fatty acid.

15. The method of claim 11, wherein the deuterated cannabigerolic acid is cyclized using tetrahydrocannabidiolic acid synthase (THCAS), cannabidiolic acid synthase (CBDAS), and/or cannabichromenic acid synthase (CBCAS).

16. A method of cell-free synthesis of a cannabinoid or derivative thereof, comprising:
cloning at least one polynucleotide sequence encoding at least one enzyme into at least one microorganism to produce a modified microorganism, wherein the modified microorganism exhibits elevated expression of the at least one enzyme in comparison to an unmodified parental microorganism;
lysing at least one cell of the modified microorganism to obtain the at least one enzyme, wherein the at least one enzyme is used in a cannabinoid biosynthesis pathway;
producing a cannabinoid or derivative thereof using the cannabinoid biosynthesis pathway, comprising;
activating a deuterated fatty acid in the presence of an acyl-activating enzyme (AAE) 3 to produce a deuterated CoA thioester, wherein the deuterated fatty acid is a compound of Formula II; 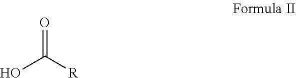
activating an acid in the presence of a corresponding CoA synthetase to produce a second CoA thioester;
synthesizing the deuterated CoA thioester and the second CoA thioester in the presence of olivetol synthase (OLS);
cyclizing the synthesized product in the presence of olivetolic acid cyclase (OAC) to produce a deuterated olivetolic acid;
prenylating the deuterated olivetolic acid in the presence of geranyl pyrophosphate to produce a deuterated cannabigerolic acid; and
cyclizing the deuterated cannabigerolic acid to produce a deuterated cannabinoid.

17. The method of claim 16, wherein the cannabinoid biosynthesis pathway is a one-pot synthesis.

18. The method of claim 16, wherein the deuterated fatty acid is a fully deuterated acid or a partially deuterated fatty acid.

19. The method of claim 16, wherein the deuterated cannabigerolic acid is cyclized using tetrahydrocannabidiolic acid synthase (THCAS), cannabidiolic acid synthase (CBDAS), and/or cannabichromenic acid synthase (CBCAS).

20. The method of claim 16, wherein the at least one enzyme is AAE3, OLS, OAC, THCAS, CBDAS, and/or CBCAS.


SEQUENCE LISTING (87 sequences)

SEQ ID NO: 1 - protein, 327 aa, synthetic construct
  REGION 1..327: note = NphB M1
MGSSHHHHHHSSGLVPRGSHMSEAADVERVYAAMEEAAGLLGVACARDKIYPLLSTFQDTLVEGGSVVVFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSFSVYPTLNWETGKIDRLCFAVISNDPTLVPSSDEGDIEKFHNYATKAPYAYVGEKRTLVYGLTLSPKEEYYKLGAAYHITDVQRGLLKAFDSLED

SEQ ID NO: 2 - protein, 327 aa, synthetic construct
  REGION 1..327: note = NphB M2
MGSSHHHHHHSSGLVPRGSHMSEAADVERVYAAMEEAAGLLGVACARDKIYPLLSTFQDTLVEGGSVVVFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSFSVYPTLNWETGKIDRLCFAVISNDPTLVPSSDEGDIEKFHNYATKAPYAYVGEKRTLVYGLTLSPKEEYYKLGANYHITDVQRGLLKAFDSLED

SEQ ID NO: 3 - protein, 327 aa, synthetic construct
  REGION 1..327: note = NphB M3
MGSSHHHHHHSSGLVPRGSHMSEAADVERVYAAMEEAAGLLGVACARDKIYPLLSTFQDTLVEGGSVVVFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSHSVYPTLNWETGKIDRLCFAVISNDPTLVPSSDEGDIEKFHNYATKAPYAYVGEKRTLVYGLTLSPKEEYYKLGAAYHITDVQRGLLKAFDSLED

SEQ ID NO: 4 - protein, 327 aa, synthetic construct
  REGION 1..327: note = NphB M4
MGSSHHHHHHSSGLVPRGSHMSEAADVERVYAAMEEAAGLLGVACARDKIYPLLSTFQDTLVEGGSVVVFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSNSVYPTLNWETGKIDRLCFAVISNDPTLVPSSDEGDIEKFHNYATKAPYAYVGEKRTLVYGLTLSPKEEYYKLGAAYHITDVQRGLLKAFDSLED

SEQ ID NO: 5 - protein, 327 aa, synthetic construct
  REGION 1..327: note = NphB M5
MGSSHHHHHHSSGLVPRGSHMSEAADVERVYAAMEEAAGLLGVACARDKIYPLLSTFQDTLVEGGSVVSFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSFSVYPTLNWETGKIDRLCFAVISNDPTLVPSSDEGDIEKFHNYATKAPYAYVGEKRTLVYGLTLSPKEEYYKLGANYHITDVQRGLLKAFDSLED

SEQ ID NO: 6 - protein, 327 aa, synthetic construct
  REGION 1..327: note = NpHB M6
MGSSHHHHHHSSGLVPRGSHMSEAADVERVYAAMEEAAGLLGVACARDKIYPLLSTFQDTLVEGGSVVNFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSFSVYPTLNWETGKIDRLCFAVISNDPTLVPSSDEGDIEKFHNYATKAPYAYVGEKRTLVYGLTLSPKEEYYKLGASYHITDVQRGLLKAFDSLED

SEQ ID NO: 7 - protein, 327 aa, synthetic construct
  REGION 1..327: note = NphB M7
MGSSHHHHHHSSGLVPRGSHMSEAADVERVYAAMEEAAGLLGVACARDKIYPLLSTFQDTLVEGGSVVSFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSFSVYPTLNWETGKIDRLCFAVISNDPTLVPSSDEGDIEKFHNYATKAPYAYVGEKRTLVYGLTLSPKEEYYKLGAAYHITDVQRGLLKAFDSLED

SEQ ID NO: 8 - protein, 327 aa, synthetic construct
  REGION 1..327: note = NphB M8
MGSSHHHHHHSSGLVPRGSHMSEAADVERVYAAMEEAAGLLGVACARDKIYPLLSTFQDTLVEGGSVVTFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSFSVYPTLNWETGKIDRLCFAVISNDPTLVPSSDEGDIEKFHNYATKAPYAYVGEKRTLVYGLTLSPKEEYYKLGANYHITDVQRGLLKAFDSLED

SEQ ID NO: 9 - protein, 327 aa, synthetic construct
  REGION 1..327: note = NphB M9
MGSSHHHHHHSSGLVPRGSHMSEAADVERVYAAMEEAAGLLGVACARDKIYPLLSTFQDTLVEGGSVVVFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSFSVYPTLNWETGKIDRLCFAVTSNDPTLVPSSDEGDIEKFHNYATKAPYAYVGEKRTLVYGLTLSPKEEYYKLGANYHITDVQRGLLKAFDSLED

SEQ ID NO: 10 - protein, 327 aa, synthetic construct
  REGION 1..327: note = NphB M10
MGSSHHHHHHSSGLVPRGSHMSEAADVERVYAAMEEAAGLLGVACARDKIYPLLSTFQDTLVEGGSVVVFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSFSVYPTLNWETGKIDRLCFAVISNDPTLVPSSDEGDIEKFHNYATKAPYAYVGEKRTLVYGLTLSPKEEYYKLSANYHITDVQRGLLKAFDSLED

SEQ ID NO: 11 - protein, 327 aa, synthetic construct
  REGION 1..327: note = NphB M11
MGSSHHHHHHSSGLVPRGSHMSEAADVERVYAAMEEAAGLLGVACARDKIYPLLSTFQDTLVEGGSVVGFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSNSVYPTLNWETGKIDRLCFAVISNDPTLVPSSDEGDIEKFHNYATKAPYAYVGEKRTLVYGLTLSPKEEYYKLGANYHITDVQRGLLKAFDSLED

SEQ ID NO: 12 - protein, 327 aa, synthetic construct
  REGION 1..327: note = NphB M12
MGSSHHHHHHSSGLVPRGSHMSEAADVERVYAAMEEAAGLLGVACARDKIYPLLSTFQDTLVEGGSVVVFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSNSVYPTLNWETGKIDRLCFAVTSNDPTLVPSSDEGDIEKFHNYATKAPYAYVGEKRTLVYGLTLSPKEEYYKLGAAYHITDVQRGLLKAFDSLED

SEQ ID NO: 13 - protein, 327 aa, synthetic construct
  REGION 1..327: note = NphB M13
MGSSHHHHHHSSGLVPRGSHMSEAADVERVYAAMEEAAGLLGVACARDKIYPLLSTFQDTLVEGGSVVNFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSNSVYPTLNWETGKIDRLCFAVISNDPTLVPSSDEGDIEKFHNYATKAPYAYVGEKRTLVYGLTLSPKEEYYKLGASYHITDVQRGLLKAFDSLED

SEQ ID NO: 14 - protein, 327 aa, synthetic construct
  REGION 1..327: note = NphB M14
MGSSHHHHHHSSGLVPRGSHMSEAADVERVYAAMEEAAGLLGVACARDKIYPLLSTFQDTLVEGGSVVVFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSGSVYPTLNWETGKIDRLCFAVTSNDPTLVPSSDEGDIEKFHNYATKAPYAYVGEKRTLVYGLTLSPKEEYYKLGANYHITDVQRGLLKAFDSLED

SEQ ID NO: 15 - protein, 327 aa, synthetic construct
  REGION 1..327: note = NphB M15
MGSSHHHHHHSSGLVPRGSHMSEAADVERVYAAMEEAAGLLGVACARDKIYPLLSTFQDTLVEGGSVVVFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSNSVYPTLNWETGKIDRLCFSVISNDPTLVPSSDEGDIEKFHNYATKAPYAYVGEKRTLVYGLTLSPKEEYYKLGAAYHITDVQRGLLKAFDSLED

SEQ ID NO: 16 - protein, 327 aa, synthetic construct
  REGION 1..327: note = NphB M16
MGSSHHHHHHSSGLVPRGSHMSEAADVERVYAAMEEAAGLLGVACARDKIYPLLSTFQDTLVEGGSVVVFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSNSVYPTLNWETGKIDRLCFSVISNDPTLVPSSDEGDIEKFHNYATKAPYAYVGEKRTLVYGLTLSPKEEYYKLGANYHITDVQRGLLKAFDSLED

SEQ ID NO: 17 - protein, 327 aa, synthetic construct
  REGION 1..327: note = NphB M17
MGSSHHHHHHSSGLVPRGSHMSEAADVERVYAAMEEAAGLLGVACARDKIYPLLSTFQDTLVEGGSVVTFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSGSVYPTLNWETGKIDRLCFAVISNDPTLVPSSDEGDIEKFHNYATKAPYAYVGEKRTLVYGLTLSPKEEYYKLGANYHITDVQRGLLKAFDSLED

SEQ ID NO: 18 - protein, 327 aa, synthetic construct
  REGION 1..327: note = NphB M18
MGSSHHHHHHSSGLVPRGSHMSEAADVERVYAAMEEAAGLLGVACARDKIYPLLSTFQDTLVEGGSVVSFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSFSVYPTLNWETGKIDRLCFAVISNDPTLVPSSDEGDIEKFHNYATKAPYAYVGEKRTLNYGLTLSPKEEYYKLGANYHITDVQRGLLKAFDSLED

SEQ ID NO: 19 - protein, 327 aa, synthetic construct
  REGION 1..327: note = NphB M19
MGSSHHHHHHSSGLVPRGSHMSEAADVERVYAAMEEAAGLLGVACARDKIYPLLSTFQDTLVEGGSVVSFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSNSVYPTLNWETGKIDRLCFAVISNDPTLVPSSDEGDIEKFHNYATKAPYAYVGEKRTLNYGLTLSPKEEYYKLGANYHITDVQRGLLKAFDSLED

SEQ ID NO: 20 - protein, 327 aa, synthetic construct
  REGION 1..327: note = NphB M20
MGSSHHHHHHSSGLVPRGSHMSEAADVERVYAAMEEAAGLLGVACARDKIYPLLSTFQDTLVEGGSVVTFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSGSVYPTLNWETGKIDRLCFAVISNDPTLVPSSDEGDIEKFHNYATKAPYAYVGEKRTLHYGLTLSPKEEYYKLGANYHITDVQRGLLKAFDSLED

SEQ ID NO: 21 - protein, 327 aa, synthetic construct
  REGION 1..327: note = NphB M21
MGSSHHHHHHSSGLVPRGSHMSEAADVERVYAAMEEAAGLLGVACARDKIYPLLSTFQDTLVEGGSVVSFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSNSVYPTLNWETGKIDRLCFSVTSNDPTLVPSSDEGDIEKFHNYATKAPYAYVGEKRTLNYGLTLSPKEEYYKLGANYHITDVQRGLLKAFDSLED

SEQ ID NO: 22 - protein, 327 aa, synthetic construct
  REGION 1..327: note = NphB M22
MGSSHHHHHHSSGLVPRGSHMSEAADVERVYAAMEEAAGLLGVACARDKIYPLLSTFQDTLVEGGSVVTFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSGSVYPTLNWETGKIDRLCFAVTSNDPTLVPSSDEGDIEKFHNYATKAPYAYVGEKRTLHYGLTLSPKEEYYKLGANYHITDVQRGILKAFDSLED

SEQ ID NO: 23 - protein, 327 aa, synthetic construct
  REGION 1..327: note = NphB M23
MGSSHHHHHHSSGLVPRGSHMSEAADVERVYAAMEEAAGLLGVACARDKIYPLLSTFQDTLVEGGSVVVFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSFSVYPTLNWETGKIDRLCFAVISNDPTLVPSSDEGDIEKFHNYATKAPYAYVGEKRTLVYGLTLSPKEEYYKLSAAYHITDVQRGLLKAFDSLED

SEQ ID NO: 24 - protein, 327 aa, synthetic construct
  REGION 1..327: note = NphB M24
MGSSHHHHHHSSGLVPRGSHMSEAADVERVYAAMEEAAGLLGVACARDKIYPLLSTFQDTLVEGGSVVVFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSFSVYPTLNWETGKIDRLCFSVISNDPTLVPSSDEGDIEKFHNYATKAPYAYVGEKRTLVYGLTLSPKEEYYKLSAAYHITDVQRGLLKAFDSLED

SEQ ID NO: 25 - protein, 327 aa, synthetic construct
  REGION 1..327: note = NphB M25
MGSSHHHHHHSSGLVPRGSHMSEAADVERVYAAMEEAAGLLGVACARDKIYPLLSTFQDTLVEGGSVVVFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSHSVYPTLNWETGKIDRLCFSVISNDPTLVPSSDEGDIEKFHNYATKAPYAYVGEKRTLVYGLTLSPKEEYYKLSAAYHITDVQRGLLKAFDSLED

SEQ ID NO: 26 - protein, 327 aa, synthetic construct
  REGION 1..327: note = NphB M27
MGSSHHHHHHSSGLVPRGSHMSEAADVERVYAAMEEAAGLLGVACARDKIYPLLSTFQDTLVEGGSVVVFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSFSVYPTLNWETGKIDRLCFAVISNDPTLVPSSDEGDIEKFHNYATKAPYAYVGEKRTLVYGLTLSPKEEYYKLSAVYHITDVQRGLLKAFDSLED

SEQ ID NO: 27 - protein, 327 aa, synthetic construct
  REGION 1..327: note = NphB M28
MGSSHHHHHHSSGLVPRGSHMSEAADVERVYAAMEEAAGLLGVACARDKIYPLLSTFQDTLVEGGSVVVFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSFSVYPTLNWETGKIDRLCFSVISNDPTLVPSSDEGDIEKFHNYATKAPYAYVGEKRTLVYGLTLSPKEEYYKLSAVYHITDVQRGLLKAFDSLED

SEQ ID NO: 28 - protein, 328 aa, synthetic construct
  REGION 1..328: note = NphB M30
MGSSHHHHHHSSGLVPRGSHMSEAADVERVYAAMEEAAGLLGVACARDKIYPLLSTFQDTLVEGGSVVVFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSFSVYPTLNWETGKIDRLCFSAVISNDPTLVPSSDEGDIEKFHNYATKAPYAYVGEKRTLVYGLTLSPKEEYYKLGAAYHITDVQRGLLKAFDSLED

SEQ ID NO: 29 - protein, 328 aa, synthetic construct
  REGION 1..328: note = NphB M31
MGSSHHHHHHSSGLVPRGSHMSEAADVERVYAAMEEAAGLLGVACARDKIYPLLSTFQDTLVEGGSVVVFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSFSVYPTLNWETGKIDRLCFSAVISNDPTLVPSSDEGDIEKFHNYATKAPYAYVGEKRTLVYGLTLSPKEEYYKLGAVYHITDVQRGLLKAFDSLED

SEQ ID NO: 30 - protein, 307 aa, Streptomyces sp. [sequence omitted]

SEQ ID NO: 31 - protein, 449 aa, Geobacillus thermodenitrificans
MTHIRFDYSKALAFFGEHELTYLRDAVKVAHHSLHEKTGVGNDFLGWLDWPVNYDKEEFARIKQAAKKIQSDSDVLLVIGIGGSYLGARAAIEMLHHSFYNALPKEKRSTPQIIFVGNNISSTYMKDVIDFLEGKDFSINVISKSGTTTEPAIAFRIFRKLLEDKYGKEEARRRIYATTDRARGALRTLADEEGYETFVIPDDIGGRYSVLTAVGLLPIAASGADIDAMMEGAAKAREDFSRSELEENAAYQYAAIRNILYNKGKTIELLVNYEPALHYFAEWWKQLFGESEGKDQKGIYPASADFSTDLHSLGQYIQEGRRDLFETVLKLEEPRHELVIEAEESDLDGLNYLAGQTVDFVNTKAFEGTLLAHTDGGVPNLVVTLPKLDEYTFGYLVYFFEKACAMSGYLLGVNPFDQPGVEAYKKNMFALLGKPGYEELKDELEKRLK

SEQ ID NO: 32 - protein, 319 aa, Geobacillus stearothermophilus
MKRIGVLTSGGDSPGMNAAIRSVVRKAIYHGVEVYGVYHGYAGLIAGNIKKLEVGDVGDIIHRGGTILYTARCPEFKTEEGQKKGIEQLKKHGIEGLVVIGGDGSYQGAKKLTEHGFPCVGVPGTIDNDIPGTDFTIGFDTALNTVIDAIDKIRDTATSHERTYVIEVMGRHAGDIALWSGLAGGAETILIPEADYDMNDVIARLKRGHERGKKHSIIIVAEGVGSGVDFGRQIQEATGFETRVTVLGHVQRGGSPTAFDRVLASRLGARAVELLLEGKGGRCVGIQNNQLVDHDIAEALANKHTIDQRMYALSKELSI

SEQ ID NO: 33 - protein, 309 aa, Escherichia coli
MVRIYTLTLAPSLDSATITPQIYPEGKLRCTAPVFEPGGGGINVARAIAHLGGSATAIFPAGGATGEHLVSLLADENVPVATVEAKDWTRQNLHVHVEASGEQYRFVMPGAALNEDEFRQLEEQVLEIESGAILVISGSLPPGVKLEKLTQLISAAQKQGIRCIVDSSGEALSAALAIGNIELVKPNQKELSALVNRELTQPDDVRKAAQEIVNSGKAKRVVVSLGPQGALGVDSENCIQVVPPPVKSQSTVGAGDSMVGAMTLKLAENASLEEMVRFGVAAGSAATLNQGTRLCSHDDTQKIYAYLSR

SEQ ID NO: 34 - protein, 319 aa, Geobacillus stearothermophilus
MKRIGVLTSGGDSPGMNAAIRSVVRKAIYHGVEVYGVYHGYAGLIAGNIKKLEVGDVGDIIHRGGTILYTARCPEFKTEEGQKKGIEQLKKHGIEGLVVIGGDGSYQGAKKLTEHGFPCVGVPGTIDNDIPGTDFTIGFDTALNTVIDAIDKIRDTATSHERTYVIEVMGRHAGDIALWSGLAGGAETILIPEADYDMNDVIARLKRGHEAGKKHSIIIVAEGVGSGVDFGRQIQEATGFETRVTVLGHVQRGGSPTAFDRVLASRLGARAVELLLEGKGGRCVGIQNNQLVDHDIAEALANKHTIDQRMYALSKELSI

SEQ ID NO: 35 - protein, 359 aa, Escherichia coli
MSKIFDFVKPGVITGDDVQKVFQVAKENNFALPAVNCVGTDSINAVLETAAKVKAPVIVQFSNGGASFIAGKGVKSDVPQGAAILGAISGAHHVHQMAEHYGVPVILHTDHCAKKLLPWIDGLLDAGEKHFAATGKPLFSSHMIDLSEESLQENIEICSKYLERMSKIGMTLEIELGCTGGEEDGVDNSHMDASALYTQPEDVDYAYTELSKISPRFTIAASFGNVHGVYKPGNVVLTPTILRDSQEYVSKKHNLPHNSLNFVFHGGSGSTAQEIKDSVSYGVVKMNIDTDTQWATWEGVLNYYKANEAYLQGQLGNPKGEDQPNKKYYDPRVWLRAGQTSMIARLEKAFQELNAIDVL

SEQ ID NO: 36 - protein, 255 aa, Escherichia coli
MRHPLVMGNWKLNGSRHMVHELVSNLRKELAGVAGCAVAIAPPEMYIDMAKREAEGSHIMLGAQNVDLNLSGAFTGETSAAMLKDIGAQYIIIGHSERRTYHKESDELIAKKFAVLKEQGLTPVLCIGETEAENEAGKTEEVCARQIDAVLKTQGAAAFEGAVIAYEPVWAIGTGKSATPAQAQAVHKFIRDHIAKVDANIAEQVIIQYGGSVNASNAAELFAQPDIDGALVGGASLKADAFAVIVKAAEAAKQA

SEQ ID NO: 37 - protein, 335 aa, Geobacillus stearothermophilus
MAVKVGINGFGRIGRNVFRAALKNPDIEVVAVNDLTDANTLAHLLKYDSVHGRLDAEVSVNGNNLVVNGKEIIVKAERDPENLAWGEIGVDIVVESTGRFTKREDAAKHLEAGAKKVIISAPAKNEDITIVMGVNQDKYDPKAHHVISNASCTTNCLAPFAKVLHEQFGIVRGMMTTVHSYTNDQRILDLPHKDLRRARAAAESIIPTTTGAAKAVALVLPELKGKLNGMAMRVPTPNVSVVDLVAELEKEVTVEEVNAALKAAAEGELKGILAYSEEPLVSRDYNGSTVSSTIDALSTMVIDGKMVKVVSWYDNETGYSHRVVDLAAYIASKGL

SEQ ID NO: 38 - protein, 335 aa, synthetic construct
  REGION 1..335: note = mGap Mutation P191D
MAVKVGINGFGRIGRNVFRAALKNPDIEVVAVNDLTDANTLAHLLKYDSVHGRLDAEVSVNGNNLVVNGKEIIVKAERDPENLAWGEIGVDIVVESTGRFTKREDAAKHLEAGAKKVIISAPAKNEDITIVMGVNQDKYDPKAHHVISNASCTTNCLAPFAKVLHEQFGIVRGMMTTVHSYTNDQRILDLDHKDLRRARAAAESIIPTTTGAAKAVALVLPELKGKLNGMAMRVPTPNVSVVDLVAELEKEVTVEEVNAALKAAAEGELKGILAYSEEPLVSRDYNGSTVSSTIDALSTMVIDGKMVKVVSWYDNETGYSHRVVDLAAYIASKGL

SEQ ID NO: 39 - protein, 335 aa, synthetic construct
  REGION 1..335: note = mGap Mutation D34A/L35R/T36K
MAVKVGINGFGRIGRNVFRAALKNPDIEVVAVNARKDANTLAHLLKYDSVHGRLDAEVSVNGNNLVVNGKEIIVKAERDPENLAWGEIGVDIVVESTGRFTKREDAAKHLEAGAKKVIISAPAKNEDITIVMGVNQDKYDPKAHHVISNASCTTNCLAPFAKVLHEQFGIVRGMMTTVHSYTNDQRILDLPHKDLRRARAAAESIIPTTTGAAKAVALVLPELKGKLNGMAMRVPTPNVSVVDLVAELEKEVTVEEVNAALKAAAEGELKGILAYSEEPLVSRDYNGSTVSSTIDALSTMVIDGKMVKVVSWYDNETGYSHRVVDLAAYIASKGL

SEQ ID NO: 40 - protein, 394 aa, Geobacillus stearothermophilus
MNKKTIRDVDVRGKRVFCRVDFNVPMEQGAITDDTRIRAALPTIRYLIEHGAKVILASHLGRPKGKVVEELRLDAVAKRLGELLERPVAKTNEAVGDEVKAAVDRLNEGDVLLLENVRFYPGEEKNDPELAKAFAELADLYVNDAFGAAHRAHASTEGIAHYLPAVAGFLMEKELEVLGKALSNPDRPFTAIIGGAKVKDKIGVIDNLLEKVDNLIIGGGLAYTFVKALGHDVGKSLLEEDKIELAKSFMEKAKEKGVRFYMPVDVVVADRFANDANTKVVPIDAIPADWSALDIGPKTRELYRDVIRESKLVVWNGPMGVFEMDAFAHGTKAIAEALAEALDTYSVIGGGDSAAAVEKFGLADKMDHISTGGGASLEFMEGKQLPGVVALEDK

SEQ ID NO: 41 - protein, 470 aa, Geobacillus thermodenitrificans
MAKQQIGVIGLAVMGKNLALNIESRGYSVAVYNRSREKTDEFLEEAKGKNIVGTYSIEEFVNALEKPRKILLMVKAGAPTDATIEQLKPYLEKGDILIDGGNTYFKDTQRRNEELAKLGIHFIGTGVSGGEEGALKGPSIMPGGQKEAHELVRPIFEAIAAKVDGEPCTTYIGPDGAGHYVKMVHNGIEYGDMQLIAEAYFLLKHVLGMDAAELHEVFADWNKGELNSYLIEITADIFTKIDDETGKPLVDVILDKAGQKGTGKWTSQNALDLGVPLPIITESVFARFISAMKDERVKASKLLSGPAVKPFEGDRDHFIEAVRRALYMSKICSYAQGFAQMKAASDEYNWNLRYGDIAMIFRGGCIIRAQFLQKIKEAYDRDPALPNLLLDPYFKNIVESYQDSLREIVATAAMRGIPVPAFASALAYYDSYRNEVLPANLIQAQRDYFGAHTYERVDKEGIFHTEWLAK

SEQ ID NO: 42 - protein, 437 aa, Escherichia coli
MSKIVKIIGREIIDSRGNPTVEAEVHLEGGFVGMAAAPSGASTGSREALELRDGDKSRFLGKGVTKAVAAVNGPIAQALIGKDAKDQAGIDKIMIDLDGTEKKSKFGANAILAVSLANAKAAAAAKGMPLYEHIAELNGTPGKYSMPVPMMNIINGGEHADNNVDIQEFMIQPVGAKTVKEAIRMGSEVFHHLAKVLKAKGMNTAVGDEGGYAPNLGSNDEALAVIAEAVKAAGYELGKDITLAMDCAASEFYKDGKYVLAGEGNKAFTSEEFTHFLEELTKQYPIVSIEDGLDESDWDGFAYQTKVLGDKIQLVGDDLFVTNTKILKEGIEKGIANSYLIKFNQIGSLTETLAAIKMAKDAGYTAVISHRSGETEDATIADLAVGTAAGQIKTGSMSRSDRVAKYNQLIRIEEALGEKARTTVVKRSKARHKTDFI

SEQ ID NO: 43 - protein, 587 aa, Geobacillus stearothermophilus
MKRKTKIVCTIGPASESVDKLVQLMEAGMNVARLNFSHGDHEEHGRRIANIREAAKRTGRTVAILLDTKGPEIRTHNMENGAIELKEGSKLVISMSEVLGTPEKISVTYPSLIDDVSVGAKILLDDGLISLEVNAVDKQAGEIVTTVLNGGVLKNKKGVNVPGVKVNLPGITEKDRADILFGIRQGIDFIAASFVRRASDVLEIRELLEAHDALHIQIIAKIENEEGVANIDEILEAADGLMVARGDLGVEIPAEEVPLIQKLLIKKCNMLGKPVITATQMLDSMQRNPRPTRAEASDVANAIFDGTDAVMLSGETAAGQYPVEAVKTMHQIALRTEQALEHRDILSQRTKESQTTITDAIGQSVAHTALNLDVAAIVTPTVSGKTPQMVAKYRPKAPIIAVTSNEAVSRRLALVWGVYTKEAPHVNTTDEMLDVAVDAAVRSGLVKHGDLVVITAGVPVGETGSTNLMKVHVISDLLAKGQGIGRKSAFGKAVVAKTAEEARQKMVDGGILVTVSTDADMMPAIEKAAAIITEEGGLTSHAAVVGLSLGIPVIVGVENATTLFKDGQEITVDGGFGAVYRGHASVL

SEQ ID NO: 44 - protein, 475 aa, Zymomonas mobilis
MTEGLFPRGRKVRVVSTLGPASSTAEQIRDRFLAGADVFRINMSHGTHDEKKVIVDNIRALEKEFNRPTTILFDLQGPKLRVGDFKEGKVQLKEGQTFTFDQDPTLGDETRVNLPHPEIFKALDKGHRLLLDDGKIVVRCVESSPTKIVTRVEVPGPLSDHKGFNVPDVVIPLAALTPKDRKDLDFALKEKADWVALSFVQRVEDVIEAKELIKGRAPLLVKLEKPAAIENLESILAATDAVMVARGDLGVECLPESVPPTQKRIVERSRQLGKPVVVATAMLESMIKAPAPTRAEVSDVANAIYEGADGIMLSAESAAGDWPHEAVNMMHRIASYVENAPGYIERVRFTPTPAEPTTVDALAENASKTAETVGAKAIIVFTETGKTAQRVSRARPVAPILSLTPDAEVARRLGLVWGAQPVQVSTVKTLDEAKKLAAETAKKYGFAKAGDKLVVVAGEPFGKAGTTNIVDVIEA

SEQ ID NO: 45 - protein, 531 aa, Oryctolagus cuniculus
MSKSHSEAGSAFIQTQQLHAAMADTFLEHMCRLDIDSAPITARNTGIICTIGPASRSVETLKEMIKSGMNVARMNFSHGTHEYHAETIKNVRTATESFASDPILYRPVAVALDTKGPEIRTGLIKGSGTAEVELKKGATLKITLDNAYMEKCDENILWLDYKNICKVVDVGSKVYVDDGLISLQVKQKGPDFLVTEVENGGFLGSKKGVNLPGAAVDLPAVSEKDIQDLKFGVEQDVDMVFASFIRKAADVHEVRKILGEKGKNIKIISKIENHEGVRRFDEILEASDGIMVARGDLGIEIPAEKVFLAQKMIIGRCNRAGKPVICATQMLESMIKKPRPTRAEGSDVANAVLDGADCIMLSGETAKGDYPLEAVRMQHLIAREAEAAMFHRKLFEELARASSHSTDLMEAMAMGSVEASYKCLAAALIVLTESGRSAHQVARYRPRAPIIAVTRNHQTARQAHLYRGIFPVVCKDPVQEAWAEDVDLRVNLAMNVGKARGFFKKGDVVIVLTGWRPGSGFTNTMRVVPVP

SEQ ID NO: 46 - protein, 592 aa, Aerococcus viridans
MSDNKINIGLAVMKILESWGADTIYGIPSGTLSSLMDAMGEEENNVKFLQVKHEEVGAMAAVMQSKFGGNLGVTVGSGGPGASHLINGLYDAAMDNIPVVAILGSRPQRELNMDAFQELNQNPMYDHIAVYNRRVAYAEQLPKLVDEAARMAIAKRGVAVLEVPGDFAKVEIDNDQWYSSANSLRKYEPIAPAAQDIDAAVELLNNSKRPVIYAGIGTMGHGPAVQELARKIKAPVITTGKNFETFEWDFEALTGSTYRVGWKPANETILEADTVLFAGSNFPFSEVEGTFRNVDNFIQIDIDPAMLGKRHHADVAILGDAGLAIDEILNKVDAVEESAWWTANLKNIANWREYINMLETKEEGDLQFYQVYNAINNHADEDAIYSIDVGNSTQTSIRHLHMTPKNMWRTSPLFATMGIAIPGGLGAKNTYPDRQVWNIIGDGAFSMTYPDVVTNVRYNMPVINVVFSNTEYAFIKNKYEDTNKNLFGVDFTDVDYAKIAEAQGAKGFTVSRIEDMDRVMAEAVAANKAGHTVVIDCKITQDRPIPVETLKLDSKLYSEDEIKAYKERYEAANLVPFREYLEAEGLESKYIK

SEQ ID NO: 47 - protein, 393 aa, Cupriavidus necator
MTDVVIVSAARTAVGKFGGSLAKIPAPELGAVVIKAALERAGVKPEQVSEVIMGQVLTAGSGQNPARQAAIKAGLPAMVPAMTINKVCGSGLKAVMLAANAIMAGDAEIVVAGGQENMSAAPHVLPGSRDGFRMGDAKLVDTMIVDGLWDVYNQYHMGITAENVAKEYGITREAQDEFAVGSQNKAEAAQKAGKFDEEIVPVLIPQRKGDPVAFKTDEFVRQGATLDSMSGLKPAFDKAGTVTAANASGLNDGAAAVVVMSAAKAKELGLTPLATIKSYANAGVDPKVMGMGPVPASKRALSRAEWTPQDLDLMEINEAFAAQALAVHQQMGWDTSKVNVNGGAIAIGHPIGASGCRILVTLLHEMKRRDAKKGLASLCIGGGMGVALAVERK

SEQ ID NO: 48 - protein, 383 aa, Enterococcus faecalis
MTIGIDKISFFVPPYYIDMTALAEARNVDPGKFHIGIGQDQMAVNPISQDIVTFAANAAEAILTKEDKEAIDMVIVGTESSIDESKAAAVVLHRLMGIQPFARSFEIKEACYGATAGLQLAKNHVALHPDKKVLVVAADIAKYGLNSGGEPTQGAGAVAMLVASEPRILALKEDNVMLTQDIYDFWRPTGHPYPMVDGPLSNETYIQSFAQVWDEHKKRTGLDFADYDALAFHIPYTKMGKKALLAKISDQTEAEQERILARYEESIIYSRRVGNLYTSSLYLGLISLLENATTLTAGNQIGLFSYGSGAVAEFFTGELVAGYQNHLQKETHLALLDNRTELSIAEYEAMFAETLDTDIDQTLEDELKYSISAINNTVRSYRN

SEQ ID NO: 49 - protein, 803 aa, Enterococcus faecalis
MKTVVIIDALRTPIGKYKGSLSQVSAVDLGTHVTTQLLKRHSTISEEIDQVIFGNVLQAGNGQNPARQIAINSGLSHEIPAMTVNEVCGSGMKAVILAKQLIQLGEAEVLIAGGIENMSQAPKLQRFNYETESYDAPFSSMMYDGLTDAFSGQAMGLTAENVAEKYHVTREEQDQFSVHSQLKAAQAQAEGIFADEIAPLEVSGTLVEKDEGIRPNSSVEKLGTLKTVFKEDGTVTAGNASTINDGASALIIASQEYAEAHGLPYLAIIRDSVEVGIDPAYMGISPIKAIQKLLARNQLTTEEIDLYEINEAFAATSIVVQRELALPEEKVNIYGGGISLGHAIGATGARLLTSLSYQLNQKEKKYGVASLCIGGGLGLAMLLERPQQKKNSRFYQMSPEERLASLLNEGQISADTKKEFENTALSSQIANHMIENQISETEVPMGVGLHLTVDETDYLVPMATEEPSVIAALSNGAKIAQGFKTVNQQRLMRGQIVFYDVADAESLIDELQVRETEIFQQAELSYPSIVKRGGGLRDLQYRAFDESFVSVDFLVDVKDAMGANIVNAMLEGVAELFREWFAEQKILFSILSNYATESVVTMKTAIPVSRLSKGSNGREIAEKIVLASRYASLDPYRAVTHNKGIMNGIEAVVLATGNDTRAVSASCHAFAVKEGRYQGLTSWTLDGEQLIGEISVPLALATVGGATKVLPKSQAAADLLAVTDAKELSRVVAAVGLAQNLAALRALVSEGIQKGHMALQARSLAMTVGATGKEVEAVAQQLKRQKTMNQDRALAILNDLRKQ

SEQ ID NO: 50 - protein, 301 aa, Methanosarcina mazei
MVSCSAPGKIYLFGEHAVVYGETAIACAVELRTRVRAELNDSITIQSQIGRTGLDFEKHPYVSAVIEKMRKSIPINGVFLTVDSDIPVGSGLGSSAAVTIASIGALNELFGFGLSLQEIAKLGHEIEIKVQGAASPTDTYVSTFGGVVTIPERRKLKTPDCGIVIGDTGVFSSTKELVANVRQLRESYPDLIEPLMTSIGKISRIGEQLVLSGDYASIGRLMNVNQGLLDALGVNILELSQLIYSARAAGAFGAKITGAGGGGCMVALTAPEKCNQVAEAVAGAGGKVTITKPTEQGLKVD

SEQ ID NO: 51 - protein, 335 aa, Streptococcus pneumoniae
MIAVKTCGKLYWAGEYAILEPGQLALIKDIPIYMRAEIAFSDSYRIYSDMFDFAVDLRPNPDYSLIQETIALMGDFLAVRGQNLRPFSLAIYGKMEREGKKFGLGSSGSVVVLVVKALLALYNLSVDQNLLFKLTSAVLLKRGDNGSMGDLACIAAEDLVLYQSFDRQKVAAWLEEENLATVLERDWGFSISQVKPTLECDFLVGWTKEVAVSSHMVQQIKQNINQNFLTSSKETVVSLVEALEQGKSEKIIEQVEVASKLLEGLSTDIYTPLLRQLKEASQDLQAVAKSSGAGGGDCGIALSFDAQSTKTLKNRWADLGIELLYQERIGHDDKS

SEQ ID NO: 52 - protein, 344 aa, Streptococcus pneumoniae
MYHSLGNQFDTRTRTSRKIRRERSCSDMDREPVTVRSYANIAIIKYWGKKKEKEMVPATSSISLTLENMYTETTLSPLPANVTADEFYINGQLQNEVEHAKMSKIIDRYRPAGEGFVRIDTQNNMPTAAGLSSSSSGLSALVKACNAYFKLGLDRSQLAQEAKFASGSSSRSFYGPLGAWDKDSGEIYPVETDLKLAMIMLVLEDKKKPISSRDGMKLCVETSTTFDDWVRQSEKDYQDMLIYLKENDFAKIGELTEKNALAMHATTKTASPAFSYLTDASYEAMDFVRQLREKGEACYFTMDAGPNVKVFCQEKDLEHLSEIFGQRYRLIVSKTKDLSQDDCC

SEQ ID NO: 53 - protein, 297 aa, Geobacillus stearothermophilus [sequence omitted]

SEQ ID NO: 54 - DNA, 789 bp, Escherichia coli
  source 1..789: mol_type = other DNA
  CDS 1..789
atgcaagtcgacctgctgggttcagcgcaatctgcgcacgcgttacacctttttcaccaacattcccctcttgtgcactgcatgaccaatgatgtggtgcaaacctttaccgccaataccttgctggcgctcggtgcatcgccagcgatggttatcgaaaccgaagaggccagtcagtttgcggctatcgccagtgccttgttgattaacgttggcacactgacgcagccacgcgctcaggcgatgcgtgctgccgttgagcaagcaaaaagctctcaaacaccctggacgcttgatccagtagcggtgggtgcgctcgattatcgccgccatttttgtcatgaacttttatcttttaaaccggcagcgatacgtggtaatgcttcggaaatcatggcattagctggcattgctaatggcggacggggagtggataccactgacgccgcagctaacgcgatacccgctgcacaaacactggcacgggaaactggcgcaatcgtcgtggtcactggcgagatggattatgttaccgatggacatcgtatcattggtattcacggtggtgatccgttaatgaccaaagtggtaggaactggctgtgcattatcggcggttgtcgctgcctgctgtgcgttaccaggcgatacgctggaaaatgtcgcatctgcctgtcactggatgaaacaagccggagaacgcgcagtcgccagaagcgaggggccaggcagttttgttccacatttccttgatgcgctctggcaattgacgcaggaggtgcaggcatga

SEQ ID NO: 55 - protein, 262 aa, Escherichia coli
MQVDLLGSAQSAHALHLFHQHSPLVHCMTNDVVQTFTANTLLALGASPAMVIETEEASQFAAIASALLINVGTLTQPRAQAMRAAVEQAKSSQTPWTLDPVAVGALDYRRHFCHELLSFKPAAIRGNASEIMALAGIANGGRGVDTTDAAANAIPAAQTLARETGAIVVVTGEMDYVTDGHRIIGIHGGDPLMTKVVGTGCALSAVVAACCALPGDTLENVASACHWMKQAGERAVARSEGPGSFVPHFLDALWQLTQEVQA

SEQ ID NO: 56 - DNA, 819 bp, Bacillus subtilis
  source 1..819: mol_type = other DNA
  CDS 1..819
atggatgcacaatcagcagcaaaatgtcttacggctgtccgccggcatagcccactggtgcatagcataaccaacaatgtcgtaacgaatttcacagcaaacggcctgctcgcgctcggcgcatcgcccgttatggcgtacgcaaaagaagaggtcgccgatatggcgaaaattgcgggtgcactcgttttaaatatcggaacactgagcaaggagtcagtcgaagcgatgatcatcgcgggaaaatcagctaatgaacatggcgttcccgtcattcttgatcctgtcggtgccggagcaacaccgttccgcactgaatcggcacgtgacatcattcgtgaggtgcgccttgctgcaatcagaggaaatgcggcggaaattgcccataccgtcggcgtgaccgattggctgatcaaaggtgttgatgcgggtgaaggtggaggcgacatcatccggctggctcagcaggcggcacaaaagctaaacacggtcattgcgataactggtgaagttgatgtcatagccgacacgtcacatgtatacacccttcataacggccacaagctgctgacaaaagtgacaggcgccggttgcctgctgacttccgtcgtcggtgcgttttgcgctgtggaagaaaatccattgtttgctgctattgcggccatttcttcgtatggggtcgccgctcagcttgccgcacagcagacggctgacaaaggccctggaagctttcagattgaattgctgaacaagctttcaactgttactgaacaagacgtccaagaatgggcgactatagaaagggtgactgtctcatga

SEQ ID NO: 57 - protein, 272 aa, Bacillus subtilis
MDAQSAAKCLTAVRRHSPLVHSITNNVVTNFTANGLLALGASPVMAYAKEEVADMAKIAGALVLNIGTLSKESVEAMIIAGKSANEHGVPVILDPVGAGATPFRTESARDIIREVRLAAIRGNAAEIAHTVGVTDWLIKGVDAGEGGGDIIRLAQQAAQKLNTVIAITGEVDVIADTSHVYTLHNGHKLLTKVTGAGCLLTSVVGAFCAVEENPLFAAIAAISSYGVAAQLAAQQTADKGPGSFQIELLNKLSTVTEQDVQEWATIERVTVS

SEQ ID NO: 58 - DNA, 783 bp, Methanocaldococcus jannaschii
  source 1..783: mol_type = other DNA
  CDS 1..783
atgttgactattcttaagttgggagggagcattctgtccgataaaaacgttccatatagcattaagtgggataacttagaacgtattgctatggaaatcaaaaacgcgttagattattacaagaaccaaaataaagaaattaagcttattctggtacatggcggcggggcatttgggcatccagtggccaagaaatacctgaagattgaagacggcaaaaaaattttcatcaacatggaaaaaggattctgggagattcagcgtgcgatgcgccgttttaataacatcatcatcgacacgcttcagagttacgatatcccagcggtctcgattcaaccttccagctttgttgtttttggcgacaaattgatcttcgacacctctgcgatcaaagagatgttgaaacgcaaccttgtacccgttatccatggggatatcgtcattgacgataaaaatgggtaccgtattatcagcggtgacgacatcgtgccatatttagccaatgaactgaaggcagatttaatcctttatgcaaccgacgtggacggcgtattgattgacaacaagcccattaaacgcattgataagaataatatctacaagattttgaattatctttcgggtagcaattcaattgacgtcacgggggggatgaaatacaagatcgacatgatccgtaaaaacaaatgccgtggtttcgtgtttaatggcaacaaggcaaacaacatttataaggcgctgcttggggaagtcgagggtaccgaaatcgacttttctgaataa

SEQ ID NO: 59 - protein, 260 aa, Methanocaldococcus jannaschii
MLTILKLGGSILSDKNVPYSIKWDNLERIAMEIKNALDYYKNQNKEIKLILVHGGGAFGHPVAKKYLKIEDGKKIFINMEKGFWEIQRAMRRFNNIIIDTLQSYDIPAVSIQPSSFVVFGDKLIFDTSAIKEMLKRNLVPVIHGDIVIDDKNGYRIISGDDIVPYLANELKADLILYATDVDGVLIDNKPIKRIDKNNIYKILNYLSGSNSIDVTGGMKYKIDMIRKNKCRGFVFNGNKANNIYKALLGEVEGTEIDFSE

SEQ ID NO: 60 - DNA, 747 bp, Methanothrix thermoacetophila
  source 1..747: mol_type = other DNA
  CDS 1..744: protein_id = 61; translation = MLKILKLGGSIITDKSRLATARLDQISRIAHEISGIENLIVVHG AGSFGHIHAKNFGLPERFSGEGLLKTHLSVSDLNRIVVEALHDAGVDALPLHPLSSVV LRDGRIHHMSTEVITEMLRRDVVPVLHGDVAMDLSKGAGIVSGDQLVSYMARTLGAGM VAMGTDVDGVMIDGRVLSCITPNDMHSLESHLLPAKGVDVTGGMRGKLAELVELAGIG IDSRIFNAGVAGNVRRALSGESLGTLITGR
atgttaaagattttgaaattgggcggtagcattattacggataagagccgcttagctactgcacgtctggatcaaatttcacgtatcgcacacgaaatctcaggcatcgagaacctgattgttgttcacggagccggttcttttggtcacatccatgccaaaaatttcggtcttccggaacgtttctcaggagaagggttactgaaaacacatctgtcggtctcggatttgaatcgtatcgtcgttgaagctcttcatgatgcaggggtggacgcgctgcccttgcaccccttatcaagtgtagtccttcgtgacggacgcatccaccatatgtctaccgaggtcattacggaaatgcttcgtcgtgatgtagtgccggtattacatggggatgttgcgatggacctgtcaaagggtgccggcattgtaagtggagaccagttggtttcgtatatggcacgtactctgggagctggtatggtcgctatggggaccgatgtcgacggggttatgatcgatggtcgtgtccttagttgcattacacctaatgacatgcactctttggagagtcacttattacccgcaaaaggggtagacgtcacgggtggaatgcgcggtaaactggcggaattagtagagctggcaggcattggaattgattcgcgtatttttaatgccggcgttgctggtaatgtacgccgtgctttgtctggggagtcgttaggaactttgattactggacgctaa

SEQ ID NO: 61 - protein, 248 aa, Methanothrix thermoacetophila
MLKILKLGGSIITDKSRLATARLDQISRIAHEISGIENLIVVHGAGSFGHIHAKNFGLPERFSGEGLLKTHLSVSDLNRIVVEALHDAGVDALPLHPLSSVVLRDGRIHHMSTEVITEMLRRDVVPVLHGDVAMDLSKGAGIVSGDQLVSYMARTLGAGMVAMGTDVDGVMIDGRVLSCITPNDMHSLESHLLPAKGVDVTGGMRGKLAELVELAGIGIDSRIFNAGVAGNVRRALSGESLGTLITGR

SEQ ID NO: 62 - DNA, 543 bp, Escherichia coli
  source 1..543: mol_type = other DNA
  CDS 1..543
atgcaaaccgagcatgtcattttattggacgagcaaggagaaccaattggaactttagaaaaatacgctgcacatacagcggacacccgcttacatcttgctttttctagttggctgtttaacgataagggtcaattattagtgacgcgccgtgcgctgagcaaaaaagcatggccgggtgtttggacgaacagtgtttgcggacacccccaactgggagaatccaatgaggatgcggtaattcgccgttgtcgctatgaattgggtgtggagattacgccaccgacaccgatctaccctgatttccgttatcgcgctacggatccttcaggtattgttgaaaatgaagtatgcccagtgtttgccgcgcgcacaacttctgcgcttcaaatcaacccagacgaggtcatggattaccaatggtgtgatcttgctgacgtactgcacgggattgacgcgacaccgtgggcttttagtccctggatggttatgcaagcgacaaatgaagaagcacgtaagcgccttcaggcgtttactcagtaa

SEQ ID NO: 63 - protein, 180 aa, Escherichia coli
MQTEHVILLDEQGEPIGTLEKYAAHTADTRLHLAFSSWLFNDKGQLLVTRRALSKKAWPGVWTNSVCGHPQLGESNEDAVIRRCRYELGVEITPPTPIYPDFRYRATDPSGIVENEVCPVFAARTTSALQINPDEVMDYQWCDLADVLHGIDATPWAFSPWMVMQATNEEARKRLQAFTQ

SEQ ID NO: 64 - DNA, 894 bp, Geobacillus stearothermophilus
  source 1..894: mol_type = other DNA
  CDS 1..894
atggcgcagctttcagttgaacagtttctcaacgagcaaaaacaggcggtggaaacagcgctctcccgttatatagagcgcttagaagggccggcgaagctgaaaaaggcgatggcgtactcattggaggccggcggcaaacgaatccgtccgttgctgcttctgtccaccgttcgggcgctcggcaaagacccggcggtcggattgcccgtcgcctgcgcgattgaaatgatccatacgtactttttgatccatgatgatttgccgagcatggacaacgatgatttgcggcgcggcaagccgacgaaccataaagtgttcggcgaggcgatggccatcttggcgggggacgggttgttgacgtacgcgtttcaattgatcaccgaaatcgacgatgagcgcatccctccttccgtccggcttcggctcatcgaacggctggcgaaagcggccggtccggaagggatggtcgccggtcaggcagccgatatggaaggagaggggaaaacgctgacgctttcggagctcgaatacattcatcggcataaaaccgggaaaatgctgcaatacagcgtgcacgccggcgccttgatcggcggcgctgatgcccggcaaacgcgggagcttgacgaattcgccgcccatctaggccttgcctttcaaattcgcgatgatattctcgatattgaaggggcagaagaaaaaatcggcaagccggtcggcagcgaccaaagcaacaacaaagcgacgtatccagcgttgctgtcgcttgccggcgcgaaggaaaagttggcgttccatatcgaggcggcgcagcgccatttacggaacgctgacgttgacggcgccgcgctcgcctatatttgcgaactggtcgccgcccgcgaccattaa

SEQ ID NO: 65 - protein, 297 aa, Geobacillus stearothermophilus
MAQLSVEQFLNEQKQAVETALSRYIERLEGPAKLKKAMAYSLEAGGKRIRPLLLLSTVRALGKDPAVGLPVACAIEMIHTYFLIHDDLPSMDNDDLRRGKPTNHKVFGEAMAILAGDGLLTYAFQLITEIDDERIPPSVRLRLIERLAKAAGPEGMVAGQAADMEGEGKTLTLSELEYIHRHKTGKMLQYSVHAGALIGGADARQTRELDEFAAHLGLAFQIRDDILDIEGAEEKIGKPVGSDQSNNKATYPALLSLAGAKEKLAFHIEAAQRHLRNADVDGAALAYICELVAARDH

SEQ ID NO: 66 - DNA, 924 bp, synthetic construct
  misc_feature 1..924: note = NphB M23 cDNA
  source 1..924: mol_type = other DNA
atgtccgaggcggccgacgtggagcgtgtttatgctgctatggaagaagctgcgggtctgctgggagtggcatgtgctcgtgataagatttatccccttctttcaaccttccaggatacattggttgaaggtggcagtgtggtagtgtttagcatggctagtggacgccacagcacggaactggactttagtatttcagtacccacgtcccatggtgacccatacgcaactgtcgtcgaaaaggggctgttccctgcaacaggccatcctgttgacgatcttttggctgatacgcagaagcacctgcctgtttctatgttcgccattgatggagaagtcaccggaggtttcaaaaaaacttatgctttctttccaactgataatatgccaggtgtggccgagttgagtgccatccccagtatgccaccggcggtcgcggaaaacgccgaattattcgcgcgttatgggttagataaggtgcagatgacgtcaatggactacaagaagcgccaggtcaatttgtacttctctgagttaagtgcacagactttagaagccgagtctgtccttgcgcttgttcgtgaactgggtttgcacgtgccgaacgaactgggtcttaaattttgcaagcgctccttttccgtttatccgacactgaactgggaaacagggaaaattgatcgtttatgttttgcggtgatttcaaacgaccctaccttggtaccaagttcggacgaaggggacattgaaaaatttcacaactacgcgacgaaggcgccgtacgcatacgtcggcgaaaagcgtacgctggtttacgggttgacgctgagtcccaaagaggaatactataaattaagcgcagcgtaccatatcaccgatgtacaacgcggactgctgaaggcctttgatagccttgaagactaa

SEQ ID NO: 67 - DNA, 924 bp, synthetic construct
  misc_feature 1..924: note = NphB M31 cDNA
  source 1..924: mol_type = other DNA
atgtccgaggcggccgacgtggagcgtgtttatgctgctatggaagaagctgcgggtctgctgggagtggcatgtgctcgtgataagatttatccccttctttcaaccttccaggatacattggttgaaggtggcagtgtggtagtgtttagcatggctagtggacgccacagcacggaactggactttagtatttcagtacccacgtcccatggtgacccatacgcaactgtcgtcgaaaaggggctgttccctgcaacaggccatcctgttgacgatcttttggctgatacgcagaagcacctgcctgtttctatgttcgccattgatggagaagtcaccggaggtttcaaaaaaacttatgctttctttccaactgataatatgccaggtgtggccgagttgagtgccatccccagtatgccaccggcggtcgcggaaaacgccgaattattcgcgcgttatgggttagataaggtgcagatgacgtcaatggactacaagaagcgccaggtcaatttgtacttctctgagttaagtgcacagactttagaagccgagtctgtccttgcgcttgttcgtgaactgggtttgcacgtgccgaacgaactgggtcttaaattttgcaagcgctccttttccgtttatccgacactgaactgggaaacagggaaaattgatcgtttatgttttagcgtgatttcaaacgaccctaccttggtaccaagttcggacgaaggggacattgaaaaatttcacaactacgcgacgaaggcgccgtacgcatacgtcggcgaaaagcgtacgctggtttacgggttgacgctgagtcccaaagaggaatactataaattaggcgcagtgtaccatatcaccgatgtacaacgcggactgctgaaggcctttgatagccttgaagactaa

SEQ ID NO: 68 - DNA, 924 bp, synthetic construct
  misc_feature 1..924: note = NpHB M31 Pross 10 cDNA
  source 1..924: mol_type = other DNA
  CDS 1..924
atgtcggaagctgccgatgtagaacgtgtctacgccgccatcgaagaagccgcaggtttgttgggggtcgcatgcgcacgcgataagatttggcccttgctgtcaacattccaggataccttggttgagggtggaagcgtagttgtttttagcatggcctcggggcgtcactcaacggagctggacttctcaatttccgtcccgcctagtcatggcgatccgtacgcgattgtggtggaaaagggcttgttcccggcaactggacatccagttgatgaccttctggcggacattcagaagcatcttcccgtatctatgtttgcgattgacggggaagttaccggggggttcaaaaaaacttatgcgttcttcccgaccgataacatgcccggtgtcgcggaactggcggccatcccatcgatgcctcctgcagtcgctgaaaatgctgaactgttcgcgcgttatggcctggacaaggtacaaatgacctcgatggattataaaaaacgtcaagtgaacctgtatttctccgaactgtcggctcagacgctggaggctgaatcagtacttgctttagtgcgtgaactgggtcttcatgtcccaaacgagctgggtctgaaattttgcaaacgctccttctcagtatacccaacattaaactgggacacctcgaagattgaccgcctttgcttctctgtaatcagtacagatccgacacttgtacctagctcagacgagggagacattgaaaaatttcacaattacgctacaaaggccccctatgcatatgttggagaaaagcgtacacttgtttacggcttgactttatctcccaaagaggagtattataaattgggtgccgtttaccacattactgacgtacaacgcaaacttttgaaggcgttcgacagccttgaggattaa

SEQ ID NO: 69 - protein, 307 aa, synthetic construct
  REGION 1..307: note = Synthetic Construct
MSEAADVERVYAAIEEAAGLLGVACARDKIWPLLSTFQDTLVEGGSVVVFSMASGRHSTELDFSISVPPSHGDPYAIVVEKGLFPATGHPVDDLLADIQKHLPVSMFAIDGEVTGGFKKTYAFFPTDNMPGVAELAAIPSMPPAVAENAELFARYGLDKVQMTSMDYKKRQVNLYFSELSAQTLEAESVLALVRELGLHVPNELGLKFCKRSFSVYPTLNWDTSKIDRLCFSVISTDPTLVPSSDEGDIEKFHNYATKAPYAYVGEKRTLVYGLTLSPKEEYYKLGAVYHITDVQRKLLKAFDSLED

SEQ ID NO: 70 - DNA, 1632 bp, Cannabis sativa
  source 1..1632: mol_type = other DNA
  CDS 1..1632
atggaaaagagtggctacggacgcgacggtatttaccgtagcctgcgtcctcctttacacctgccaaacaataacaatttgagtatggtctcattcctgttccgtaacagcagcagctatccacagaaaccggcgttgatcgatagcgagactaatcaaattttatcttttagtcattttaaaagcaccgtgatcaaggtctcccatggcttcttaaacctggggatcaaaaagaatgacgtggttttaatctacgcacccaattcgatccactttcccgtatgcttccttggcattattgcttctggggcgatcgccactacttcaaatccattatacaccgtgagtgagttgtcgaaacaagtaaaggactcgaaccctaaattgattatcacagtccctcagttattggaaaaggtcaagggtttcaatctgccaactatccttatcggccctgattctgagcaggaatcgtctagtgataaagtaatgactttcaatgatctggtcaatctgggaggaagttcgggtagcgaattccctatcgtcgacgatttcaagcaatccgacaccgccgcactgttgtactcaagtggcacgacaggtatgagcaagggggtcgttctgacgcacaaaaattttattgcctcatcgttgatggtaacaatggaacaggacttggtcggcgagatggacaatgtgttcctgtgtttccttcctatgtttcacgtctttggcttagccattattacgtatgctcagttacagcgcggtaataccgtgatttcaatggcccgctttgacttggaaaagatgttaaaagatgttgaaaagtacaaagttacccacctttgggtcgtacccccagttatcttagcgttgtcgaagaactcaatggtgaaaaaattcaatttgtcatccatcaagtatattggttcaggcgctgcgccattaggaaaggatctgatggaagaatgctctaaggtggttccttacggaatcgtggctcaaggatatggcatgacggaaacgtgcggaatcgtatccatggaagacatccgcggcgggaaacgcaattcagggtcggccggaatgttggcaagtggggtagaagctcagatcgtgagtgtggacaccttaaaaccccttcccccgaatcaattaggggaaatctgggtaaaaggtccaaatatgatgcaaggctatttcaacaatcctcaagcgaccaaacttaccattgataaaaagggttgggttcatactggcgacttggggtatttcgacgaagacggacacttatatgttgtagaccgtattaaggagcttattaaatacaagggattccaagttgcgcctgcggaactggagggattattagttagtcaccccgagatcttagacgcggtagttattcccttccccgatgctgaggcaggcgaagtcccggtggcatacgttgttcgctcgcctaacagttcgttgaccgaaaatgacgttaaaaaattcatcgccggtcaggtcgcctcctttaagcgtctgcgcaaggttacttttattaattccgtccccaagagcgcaagtgggaagattctgcgccgcgagcttattcaaaaggttcgctctaacatgtaa

SEQ ID NO: 71 - protein, 543 aa, Cannabis sativa
MEKSGYGRDGIYRSLRPPLHLPNNNNLSMVSFLFRNSSSYPQKPALIDSETNQILSFSHFKSTVIKVSHGFLNLGIKKNDVVLIYAPNSIHFPVCFLGIIASGAIATTSNPLYTVSELSKQVKDSNPKLIITVPQLLEKVKGFNLPTILIGPDSEQESSSDKVMTFNDLVNLGGSSGSEFPIVDDFKQSDTAALLYSSGTTGMSKGVVLTHKNFIASSLMVTMEQDLVGEMDNVFLCFLPMFHVFGLAIITYAQLQRGNTVISMARFDLEKMLKDVEKYKVTHLWVVPPVILALSKNSMVKKFNLSSIKYIGSGAAPLGKDLMEECSKVVPYGIVAQGYGMTETCGIVSMEDIRGGKRNSGSAGMLASGVEAQIVSVDTLKPLPPNQLGEIWVKGPNMMQGYFNNPQATKLTIDKKGWVHTGDLGYFDEDGHLYVVDRIKELIKYKGFQVAPAELEGLLVSHPEILDAVVIPFPDAEAGEVPVAYVVRSPNSSLTENDVKKFIAGQVASFKRLRKVTFINSVPKSASGKILRRELIQKVRSNM

SEQ ID NO: 72 - DNA, 2163 bp, Cannabis sativa
  source 1..2163: mol_type = other DNA
  CDS 1..2163
atgggtaagaattacaagtccctggactctgttgtggcctctgacttcatagccctaggtatcacctctgaagttgctgagacactccatggtagactggccgagatcgtgtgtaattatggcgctgccactccccaaacatggatcaatattgccaaccatattctgtcgcctgacctccccttctccctgcaccagatgctcttctatggttgctataaagactttggacctgcccctcctgcttggatacccgacccggagaaagtaaagtccaccaatctgggcgcacttttggagaagcgaggaaaagagtttttgggagtcaagtataaggatcccatttcaagcttttctcatttccaagaattttctgtaagaaaccctgaggtgtattggagaacagtactaatggatgagatgaagataagtttttcaaaggatccagaatgtatattgcgtagagatgatattaataatccagggggtagtgaatggcttccaggaggttatcttaactcagcaaagaattgcttgaatgtaaatagtaacaagaaattgaatgatacaatgattgtatggcgtgatgaaggaaatgatgatttgcctctaaacaaattgacacttgaccaattgcgtaaacgtgtttggttagttggttatgcacttgaagaaatgggtttggagaagggttgtgcaattgcaattgatatgccaatgcatgtggatgctgtggttatctatctagctattgttcttgcgggatatgtagttgtttctattgctgatagtttttctgctcctgaaatatcaacaagacttcgactatcaaaagcaaaagccatttttacacaggatcatattattcgtgggaagaagcgtattcccttatacagtagagttgtggaagccaagtctcccatggccattgttattccttgtagtggctctaatattggtgcagaattgcgtgatggcgatatttcttgggattactttctagaaagagcaaaagagtttaaaaattgtgaatttactgctagagaacaaccagttgatgcctatacaaacatcctcttctcatctggaacaacaggggagccaaaggcaattccatggactcaagcaactcctttaaaagcagctgcagatgggtggagccatttggacattaggaaaggtgatgtcattgtttggcccactaatcttggttggatgatgggtccttggctggtctatgcttcactccttaatggggcttctattgccttgtataatggatcaccacttgtttctggctttgccaaatttgtgcaggatgctaaagtaacaatgctaggtgtggtccctagtattgttcgatcatggaaaagtaccaattgtgttagtggctatgattggtccaccatccgttgcttttcctcttctggtgaagcatctaatgtagatgaatacctatggttgatggggagagcaaactacaagcctgttatcgaaatgtgtggtggcacagaaattggtggtgcattttctgctggctctttcttacaagctcaatcattatcttcatttagttcacaatgtatgggttgcactttatacatacttgacaagaatggttatccaatgcctaaaaacaaaccaggaattggtgaattagcgcttggtccagtcatgtttggagcatcgaagactctgttgaatggtaatcaccatgatgtttattttaagggaatgcctacattgaatggagaggttttaaggaggcatggggacatttttgagcttacatctaatggttattatcatgcacatggtcgtgcagatgatacaatgaatattggaggcatcaagattagttccatagagattgaacgagtttgtaatgaagttgatgacagagttttcgagacaactgctattggagtgccacctttgggcggtggacctgagcaattagtaattttctttgtattaaaagattcaaatgatacaactattgacttaaatcaattgaggttatctttcaacttgggtttacagaagaaactaaatcctctgttcaaggtcactcgtgttgtgcctctttcatcacttccgagaacagcaaccaacaagatcatgagaagggttttgcgccagcaattttctcactttgaatga

SEQ ID NO: 73 - protein, 720 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 74 - DNA, 1695 bp, Cannabis sativa
  source 1..1695: mol_type = other DNA
  CDS 1..1695
atggaagtactgaaggaggttgcgaaggaaggtagcgcagcccgtgaaggtgtcgctattcgcgccgaccagaaatcgtacagctataagcaattgatctcctccgcgcagtcgatctgctcactgttatgcggtactgaacttaaagcgattcacgaagccgggaaacaagctcgtcctagcgcgtctatcaatggggccgggggtcacggccacttgggaggagctcgtattggaattgttgctaagccgtcggcagaatttgtagccggtgttttaggtacgtggttatctggtggagttgcggttccccttgcactgtcttacccggaggcggaattactgcatgtcatgaacgattcagatatcagcatgatcttgagcacggaagaccatcaagaactgatgcaaaatattgctgccaagacttccgcacagttttccttaattccatctgtgccgtcgtcgtgctcacaagaagtagcggtcgatcatcgtcagaccggtgacatctctaccgactctatcttgcttaaccgcgagatctctaacgagaatcccgcacttatcgtctatacgtcggggacgacaggcaagccgaagggcgtcgttcacacacaccaatcaatttctgcacaggttcagacgttagccaaggcatgggagtatactcctgccgatcaattcttacactgcttaccgctgcatcatgtgcatgggctgtttaacgcactgttcgcgcccctttacgcgcgttcaacagttgaatttctgccgaaattttctgtccgcggtatttggcaacgctggcgcgaatcctacccaacgtcagagacgaaagccaatgactgcattacggtatttacaggagttcccaccatgtacacgcgtctgattcaaggatatgaagctatggatccagagttaaaagaggcctctgcatctgctgctaagcagctgcgccttatgatgtgtggttcctctgcgctgccagttcctgtcatgcagcagtggcaaaccatcaccggccaccgtcttctggaacgttacggaatgaccgaatttgtcatggcaatttctaaccccttgaaaggtgagcgcaaatccggtactgtcggaaagccgtttccaggtgtagaggtgcgcattttagcagaggatgaaaacggcgatgatgctaccggggtgggagagctgtgcgtacgcagtccgtcccttttcaaagagtattggcgtttgcccgaggtcacaaaagcctcctttacagacgacggctttttcaaaaccggcgacgcaggcaaggtcgatgaggacggttactacgtgattctgggccgtactagcgcagatattatgaaagttggaggctataagctgtctgctctggaaatcgagtcggtccttctggaacacccgactgtcgaggaatgctgtgtcttgggacttcccgacaaggattatggggaagccgtatccgcaatcattgtaccggcagccgaggcgaagaagaaacgcgaagaggagtcacgccccgccattagtctggaggaactgttctcatgggcacagcacaaacttgccccctacaaactgcccacgcgtttattcctgtgggactctttacctcgcaacgcaatggggaaagtcaacaaaaaagagctgaagaaaaaactgacagttgagcaaggtatttaa

SEQ ID NO: 75 - protein, 564 aa, Cannabis sativa
MEVLKEVAKEGSAAREGVAIRADQKSYSYKQLISSAQSICSLLCGTELKAIHEAGKQARPSASINGAGGHGHLGGARIGIVAKPSAEFVAGVLGTWLSGGVAVPLALSYPEAELLHVMNDSDISMILSTEDHQELMQNIAAKTSAQFSLIPSVPSSCSQEVAVDHRQTGDISTDSILLNREISNENPALIVYTSGTTGKPKGVVHTHQSISAQVQTLAKAWEYTPADQFLHCLPLHHVHGLFNALFAPLYARSTVEFLPKFSVRGIWQRWRESYPTSETKANDCITVFTGVPTMYTRLIQGYEAMDPELKEASASAAKQLRLMMCGSSALPVPVMQQWQTITGHRLLERYGMTEFVMAISNPLKGERKSGTVGKPFPGVEVRILAEDENGDDATGVGELCVRSPSLFKEYWRLPEVTKASFTDDGFFKTGDAGKVDEDGYYVILGRTSADIMKVGGYKLSALEIESVLLEHPTVEECCVLGLPDKDYGEAVSAIIVPAAEAKKKREEESRPAISLEELFSWAQHKLAPYKLPTRLFLWDSLPRNAMGKVNKKELKKKLTVEQGI

SEQ ID NO: 76 - DNA, 1158 bp, Cannabis sativa
  source 1..1158: mol_type = other DNA
  CDS 1..1158
atgaatcatctgcgtgctgaaggaccagcttccgtattggcaattggaacagctaaccctgagaacattcttcttcaggatgagtttcccgactattacttccgcgtgacaaagagcgaacacatgacacagcttaaagagaagttccgtaagatctgtgacaaaagcatgatccgcaaacgtaactgcttccttaacgaggagcatctgaagcagaatccccgtcttgttgaacatgagatgcagaccttggatgctcgccaggacatgttggttgttgaggtccctaagctgggcaaagatgcgtgtgcaaaagcgattaaagagtgggggcagcctaaaagcaaaattactcatctgattttcacaagcgccagtacaaccgatatgcccggtgcggactaccattgtgcaaaattattgggtttatcgccttcagtaaaacgtgttatgatgtaccagttaggatgctacggtggtggcaccgtacttcgtattgcgaaggacatcgccgagaacaacaaaggagcccgtgtacttgctgtatgttgtgatatcatggcgtgcctttttcgcggccccagcgagagtgaccttgagttacttgtggggcaggccatcttcggagacggtgccgcagccgtcattgttggcgcagagcccgatgaatccgttggcgagcgcccgatctttgagcttgtaagtacaggacaaactatcttgcccaactctgaggggactatcggcggacatattcgtgaggcgggcttgatttttgaccttcacaaggatgttccaatgcttatctccaataatattgaaaaatgtcttatcgaagcattcactccgattggtatctccgattggaattcgattttttggatcacccatcctggtgggaaagctattttagacaaggtggaggagaaattacatcttaagtcagataagtttgtcgacagtcgccacgtgttgtcggaacatggcaacatgtcatcgtcaaccgtcttgttcgttatggacgaattacgtaaacgcagtttagaagagggtaagagtacgacgggggacgggttcgagtggggagtcttattcgggttcggtccaggattgacagtggaacgcgtcgtggttcgcagtgtccccattaagtactaa

SEQ ID NO: 77 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 78 - DNA, 306 bp, Cannabis sativa
  source 1..306: mol_type = other DNA
  CDS 1..306
atggcagtcaaacacttgatcgtgttaaagttcaaagatgaaatcacagaggctcagaaggaagaatttttcaagacgtatgtaaaccttgttaatatcatccccgctatgaaggatgtgtattggggtaaagacgtgacacagaagaacaaagaggaaggctacacgcacatcgtagaggtcacatttgagagcgtcgaaactattcaggattacatcattcatcccgcacacgttggattcggggatgtgtatcgctctttctgggaaaaattgctgatcttcgactatacaccgcgtaagtaa

SEQ ID NO: 79 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 80 - DNA, 654 bp, Geobacillus thermodenitrificans
  source 1..654: mol_type = other DNA
  CDS 1..654
atgaatttagtgctgatggggctgccaggtgccggcaaaggcacgcaagccgagaaaatcgtagaaacatatggaatcccacatatttcaaccggggatatgtttcgggcggcgatgaaagaaggcacaccgttaggattgcaggcaaaagaatatatcgaccgtggtgatcttgttccggatgaggtgacgatcggtatcgtccgtgaacggttaagcaaagacgactgccaaaacggctttttgcttgacggattcccacgcacggttgcccaagcggaggcgctggaagcgatgctggctgaaatcggccgcaagcttgactatgtcatccatatcgatgttcgccaagatgtgttaatggagcgcctcacaggcagacgaatttgtcgcaactgcggagcgacataccatcttgtttttcacccaccggctcagccaggcgtatgtgataaatgcggtggcgagctttatcagcgccctgacgataatgaagcaacagtggcgaatcggcttgaggtgaatacgaaacaaatgaagccattgctcgatttctatgagcaaaaaggctatttgcgtcacattaacggcgaacaagaaatggaaaaagtgtttagcgacattcgcgaattgctcgggggacttactcgataa

SEQ ID NO: 81 - protein, 217 aa, Geobacillus thermodenitrificans
MNLVLMGLPGAGKGTQAEKIVETYGIPHISTGDMFRAAMKEGTPLGLQAKEYIDRGDLVPDEVTIGIVRERLSKDDCQNGFLLDGFPRTVAQAEALEAMLAEIGRKLDYVIHIDVRQDVLMERLTGRRICRNCGATYHLVFHPPAQPGVCDKCGGELYQRPDDNEATVANRLEVNTKQMKPLLDFYEQKGYLRHINGEQEMEKVFSDIRELLGGLTR

SEQ ID NO: 82 - DNA, 1512 bp, Rhodopseudomonas palustris
  source 1..1512: mol_type = other DNA
  CDS 1..1512
atgaacgccaacctgttcgcccgcctgttcgataagctcgacgacccccacaagctcgcgatcgaaaccgcggccggggacaagatcagctacgccgagctggtggcgcgggcgggccgcgtcgccaacgtgctggtggcacgcggcctgcaggtcggcgaccgcgttgcggcgcaaaccgagaagtcggtggaagcgctggtgctgtatctcgccacggtgcgggccggcggcgtgtatctgccgctcaacaccgcctatacgctgcacgagctcgattacttcatcaccgatgccgagccgaagatcgtggtgtgcgatccgtccaagcgcgacgggatcgcggcgattgccgccaaggtcggcgccacggtggagacgcttggccccgacggtcggggctcgctcaccgatgcggcagctggagccagcgaggcgttcgccacgatcgaccgcggcgccgatgatctggcggcgatcctctacacctcagggacgaccggccgctccaagggcgcgatgctcagccacgacaatttggcgtcgaactcgctgacgctggtcgattactggcgcttcacgccggatgacgtgctgatccacgcgctgccgatctatcacacccatggattgttcgtggccagcaacgtcacgctgttcgcgcgcggatcgatgatcttcctgccgaagttcgatcccgacaagatcctcgacctgatggcgcgcgccaccgtgctgatgggtgtgccgacgttctacacgcggctcttgcagagcccgcggctgaccaaggagacgacgggccacatgaggctgttcatctccgggtcggcgccgctgctcgccgatacgcatcgcgaatggtcggcgaagaccggtcacgccgtgctcgagcgctacggcatgaccgagaccaacatgaacacctcgaacccgtatgacggcgaccgcgtccccggcgcggtcggcccggcgctgcccggcgtttcggcgcgcgtgaccgatccggaaaccggcaaggaactgccgcgcggcgacatcgggatgatcgaggtgaagggcccgaacgtgttcaagggctactggcggatgccggagaagaccaagtctgaattccgcgacgacggcttcttcatcaccggcgacctcggcaagatcgacgagcgcggctacgtccacatcctcggccgcggcaaggatctggtgatcaccggcggcttcaacgtctatccgaaggaaatcgagagcgagatcgacgccatgccgggcgtggtcgaatccgcggtgatcggcgtgccgcacgccgatttcggcgagggcgtcactgccgtggtggtgcgcgacaagggtgccacgatcgacgaagcgcaggtgctgcacggcctcgacggtcagctcgccaagttcaagatgccgaagaaagtgatcttcgtcgacgacctgccgcgcaacaccatgggcaaggtccagaagaacgtcctgcgcgagacctacaaggacatctacaagtaa

SEQ ID NO: 83 - protein, 503 aa, Rhodopseudomonas palustris
MNANLFARLFDKLDDPHKLAIETAAGDKISYAELVARAGRVANVLVARGLQVGDRVAAQTEKSVEALVLYLATVRAGGVYLPLNTAYTLHELDYFITDAEPKIVVCDPSKRDGIAAIAAKVGATVETLGPDGRGSLTDAAAGASEAFATIDRGADDLAAILYTSGTTGRSKGAMLSHDNLASNSLTLVDYWRFTPDDVLIHALPIYHTHGLFVASNVTLFARGSMIFLPKFDPDKILDLMARATVLMGVPTFYTRLLQSPRLTKETTGHMRLFISGSAPLLADTHREWSAKTGHAVLERYGMTETNMNTSNPYDGDRVPGAVGPALPGVSARVTDPETGKELPRGDIGMIEVKGPNVFKGYWRMPEKTKSEFRDDGFFITGDLGKIDERGYVHILGRGKDLVITGGFNVYPKEIESEIDAMPGVVESAVIGVPHADFGEGVTAVVVRDKGATIDEAQVLHGLDGQLAKFKMPKKVIFVDDLPRNTMGKVQKNVLRETYKDIYK

SEQ ID NO: 84 - protein, 405 aa, Thermococcus kodakarensis
MAGKVRNIVVEELVRTPVEMQKVELVERKGIGHPDSIADGIAEAVSRALSREYVKRYGIILHHNTDQVEVVGGRAYPQFGGGEVIKPIYILLSGRAVEMVDREFFPVHEIALKAAKDYLRKAVRHLDLEHHVIIDSRIGQGSVDLVGVFNKAKKNPIPLANDTSFGVGYAPLSETEKIVLETEKYLNSDEFKKKYPAVGEDIKVMGLRKGDEIDLTIAAAIVDSEVDNPDDYMAVKEAIYEAAKGIVESHTERPTNIYVNTADDPKEGIYYITVTGTSAEAGDDGSVGRGNRVNGLITPNRHMSMEAAAGKNPVSHVGKIYNILSMLIANDIAEQVEGVEEVYVRILSQIGKPIDEPLVASVQIIPKKGYSIDVLQKPAYEIADEWLANITKIQKMILEDKVNVF

SEQ ID NO: 85 - DNA, 1218 bp, Thermococcus kodakarensis
  source 1..1218: mol_type = other DNA
atggcgggcaaagtacgcaatatcgtagtagaggagctggtccgtacgcccgtcgagatgcagaaggtagagctggtagagcgcaaagggatcggacacccagattcgattgccgacggcattgctgaagcggtttcacgtgcgttgagtcgtgaatatgtaaagcgctatggaattattttgcaccacaatacggatcaagtcgaagttgtgggtgggcgtgcgtacccacaatttggaggtggagaagtcattaaaccgatttacatcctgttgagcggtcgtgccgttgaaatggtggaccgtgagtttttccctgtccacgagattgctttaaaagctgcaaaggattaccttcgcaaagccgtgcgccatttagatttagagcaccatgtgattattgactcacgtattggccaaggatcagtcgatcttgtaggtgtgttcaataaagccaagaagaaccctatcccactggcgaatgatacatcttttggagttggttatgccccgctgtctgagactgagaagatcgttttagaaaccgagaaataccttaattcagatgaattcaaaaaaaagtatccagcagtcggcgaagacatcaaagttatggggttgcgtaagggtgatgaaattgacctgactattgcagcggccattgtagatagtgaggtagataatcccgatgattatatggcggtcaaagaggccatctacgaagctgcaaagggcattgtggaatctcatacggagcgtcccaccaatatctacgtcaacacagctgacgaccctaaagagggcatctattacatcactgttaccggcacgagtgccgaagctggagatgatgggagcgtcggacgtgggaaccgcgtcaatgggttaattacacctaatcgtcacatgtctatggaagccgccgcaggcaagaacccggttagtcatgtcggaaagatttacaatattctgtctatgttaattgccaatgacattgcggagcaagtagaaggtgtcgaggaggtgtacgtacgcatcctgtcgcaaatcggaaagccgattgatgagccgttagtagccagcgtgcaaatcattccgaaaaagggatactcgattgatgtgttacagaagccggcatacgaaatcgcggacgaatggctggcgaatatcactaagattcagaagatgattctggaggataaggtcaatgtgttttaa

SEQ ID NO: 86 - protein, 406 aa, Methanocaldococcus jannaschii
MRNIIVKKLDVEPIEERPTEIVERKGLGHPDSICDGIAESVSRALCKMYMEKFGTILHHNTDQVELVGGHAYPKFGGGVMVSPIYILLSGRATMEILDKEKNEVIKLPVGTTAVKAAKEYLKKVLRNVDVDKDVIIDCRIGQGSMDLVDVFERQKNEVPLANDTSFGVGYAPLSTTERLVLETERFLNSDELKNEIPAVGEDIKVMGLREGKKITLTIAMAVVDRYVKNIEEYKEVIEKVRKKVEDLAKKIADGYEVEIHINTADDYERESVYLTVTGTSAEMGDDGSVGRGNRVNGLITPFRPMSMEAASGKNPVNHVGKIYNILANLIANDIAKLEGVKECYVRILSQIGKPINEPKALDIEIITEDSYDIKDIEPKAKEIANKWLDNIMEVQKMIVEGKVTTF

SEQ ID NO: 87 - DNA, 1221 bp, Methanocaldococcus jannaschii
  source 1..1221: mol_type = other DNA
atgcgtaatatcatcgttaagaagttagatgtcgaaccaattgaagaacgcccgacagagattgtggaacgcaaggggctggggcatcctgattcaatctgtgacggcatcgctgaatcagtaagtcgcgcgttgtgcaaaatgtacatggaaaagtttggtacgattttgcatcataatacggatcaagtggagctggtcgggggtcatgcgtatcccaagttcggaggaggcgtcatggtctcacccatctatattcttttatcaggacgtgcaactatggaaatccttgataaagaaaagaacgaagtcattaagctgcccgtcggaactactgcagtaaaggctgcgaaggagtacttaaaaaaagtccttcgtaacgttgacgtagacaaagacgttatcattgattgccgtattggtcaggggtctatggaccttgtagacgtgttcgagcgtcaaaaaaacgaggtccccttggccaatgatacttcttttggagttggctacgcaccgctgtcaaccaccgaacgtcttgtattggagacagaacgcttcttgaactcggatgaattgaaaaacgagatcccagcggtgggtgaagacatcaaggttatggggttacgcgagggcaaaaagattacacttacaattgcaatggctgttgtggaccgctatgtaaagaacattgaagaatataaagaagtgattgaaaaggtccgcaaaaaggttgaagaccttgcgaaaaaaatcgcagacggttacgaagttgagattcatatcaacacagctgacgactacgaacgtgagtctgtatatctgactgtcacggggacgagtgccgagatgggtgacgacggatcggtaggtcgtggcaatcgtgtaaacggtcttatcactccctttcgtccaatgtcgatggaggcagcttctggaaagaatccggtgaatcacgttgggaaaatctacaacattttggcgaatctgatcgccaacgatatcgcaaagctggagggggttaaagaatgttacgtccgcatccttagtcaaattgggaagcccattaacgagccgaaagctctggatattgagatcattacggaggatagctatgacatcaaagacattgaaccaaaggcgaaggaaattgcaaacaaatggctggataacattatggaggtacagaaaatgatcgttgaaggaaaggtcacgactttctaa